(19) United States
(12) Patent Application Publication
(10) Pub. No.: US 2005/0233956 A1
(43) Pub. Date: Oct. 20, 2005

(54) Proteins involved in the regulation of energy homeostasis

(75) Inventors: Karsten Eulenberg, Bovenden (DE); Steuernagel Arnd, Gottingen (DE); Hader Thomas, Gottingen (DE); Meise Martin, Gottingen (DE); Bronner Gunter, Gottingen (DE)

Correspondence Address: ROTHWELL, FIGG, ERNST & MANBECK, P.C., 1425 K STREET, N.W., SUITE 800, WASHINGTON, DC, 20005, US

(73) Assignee: DeveloGen Aktiengesellschaft Fur Entwicklungsbiologische Forschung, Goettingen (DE)

(21) Appl. No.: 10/511,627

(22) PCT Filed: May 2, 2003
(86) PCT No.: PCT/EP03/04650

§ 371 Date: Oct. 18, 2004

(30) Foreign Application Priority Data
May 2, 2002 (EP) 02009883.6
May 7, 2002 (EP) 02010332.1
May 16, 2002 (EP) 02010948.1

(51) Int. Cl. C12Q 1/68; G01N 33/53; G01N 33/567; A01K 67/00; C07K 14/705; A61K 48/00; C12N 5/08

(52) U.S. Cl. 514/12; 514/44; 435/6; 435/7.2; 435/320.1; 435/366; 800/8; 530/350; 536/23.2

(57) ABSTRACT

The present invention discloses novel uses for energy homeostasis regulating proteins and polynucleotides encoding these in the diagnosis, study, prevention, and treatment of metabolic diseases and disorders.

This invention relates to the use of CG7956, aralar1, how (held out wings), CG9373, cpo (couch potato), Jafrac1 (thioredoxin peroxidase 1), or CG14440 homologous proteins, to the use of polynucleotides encoding these, and to the use of effectors/modulators of the proteins and polynucleotides in the diagnosis, study, prevention, and treatment of obesity and/or diabetes and/or metabolic syndrome.

There are several metabolic diseases of human and animal metabolism, eg., obesity and severe weight loss, that relate to energy imbalance where caloric intake versus energy expediture is imbalanced. Obesity is one of the most prevalent metabolic disorders in the world. It is still a poorly understood human disease that becomes as a major health problem more and more relevant for western society. Obesity is defined as a body weight more than 20% in excess of the ideal body weight, frequently resulting in a significant impairment of health. Obesity may be measured by body mass index, an indicator of adiposity or fatness. Further parameters for defining obesity are waist circumferences, skinfold thickness and bioimpedance (see, inter alia, Kopelman (1999), loc. cit.). Obesity is associated with an increased risk for cardiovascular disease, hypertension, diabetes, hyperlipidaemia and an increased mortality rate. Besides severe risks of illness, individuals suffering from obesity are often isolated socially.

Obesity is influenced by genetic, metabolic, biochemical, psychological, and behavioral factors, and can be caused by different reasons such as non-insulin dependent diabetes, increase in triglycerides, increase in carbohydrate bound energy and low energy expenditure. As such, it is a complex disorder that must be addressed on several fronts to achieve lasting positive clinical outcome. Since obesity is not to be considered as a single disorder but as a heterogeneous group of conditions with (potential) multiple causes, it is also characterized by elevated fasting plasma insulin and an exaggerated insulin response to oral glucose intake (Koltermann J., (1980) Clin. Invest 65, 1272-1284). A clear involvement of obesity in type 2 diabetes mellitus can be confirmed (Kopelman P. G., (2000) Nature 404, 635-643).

Hyperlipidemia and elevation of free fatty acids correlate clearly with the metabolic syndrome, which is defined as the linkage between several diseases, including obesity and insulin resistance. This often occurs in the same patients and are major risk factors for development of type 2 diabetes and cardiovascular disease. It was suggested that the control of lipid levels and glucose levels is required to treat type 2 diabetes, heart disease, and other occurances of metabolic syndrome (see, for example, Santomauro A. T. et al., (1999) Diabetes, 48(9):1836-1841 and McCook, 2002, JAMA 288:2709-2716).

The molecular factors regulating food intake and body weight balance are incompletely understood. Even if several candidate genes have been described which are supposed to influence the homeostatic system(s) that regulate body mass/weight, like leptin or the peroxisome proliferator-activated receptor-gamma co-activator, the distinct molecular mechanisms and/or molecules influencing obesity or body weight/body mass regulations are not known. In addition, several single-gene mutations resulting in obesity have been described in mice, implicating genetic factors in the etiology of obesity (Friedman and Leibel, 1990, Cell 69: 217-220). In the ob mouse a single gene mutation (obese) results in profound obesity, which is accompanied by diabetes (Friedman et. al., 1991, Genomics 11: 1054-1062).

Therefore, the technical problem underlying the present invention was to provide for means and methods for modulating (pathological) metabolic conditions influencing body-weight regulation and/or energy homeostatic circuits. The solution to said technical problem is achieved by providing the embodiments characterized in the claims. Accordingly, the present invention relates to novel functions of proteins and nucleic acids encoding these in body-weight regulation, energy homeostasis, metabolism, and obesity. The proteins disclosed herein and polynucleotides encoding these are thus suitable to investigate metabolic diseases and disorders. Further new compositions are provided that are useful in diagnosis, treatment, and prognosis of metabolic diseases and disorders as described.

KIAA0966 encodes for a Synaptojanin-like protein, the Sac domain-containing inositol phosphatase (hSac2). Synaptic vesicles are recycled with remarkable speed and precision in nerve terminals. A major recycling pathway involves clathrin-mediated endocytosis at endocytic zones located around sites of release. Different ‘accessory’ proteins linked to this pathway have been shown to alter the shape and composition of lipid membranes, to modify membrane-coat protein interactions, and to influence actin polymerization. These include the GTPase dynamin, the lysophosphatidic acid acyl transferase endophilin, and the phosphoinositide phosphatase synaptojanin (Brodin L. et al., 2000, Curr Opin Neurobiol 10(3):312-320). Studies on the endocytosis of synaptic vesicles have shown the essential roles of endophilin and synaptojanin in vesicle formation (see, Ringstad N. et al., 1999, Neuron 24(1):143-154). The recessive suppressor of secretory defect in yeast Golgi and yeast actin function belongs to this family (Luo W. and Chang A., 1997, J Cell Biol 138(4):731-746). This protein may be involved in the coordination of the activities of the secretory pathway and the actin cytoskeleton. Human synaptojanin, which may be localised on coated endocytic intermediates in nerve terminals also belongs to this family (Haffner C. et al., 1997, FEBS Lett 419(2-3):175-180). Studies on the endocytosis of synaptic vesicles have shown the essential roles of endophilin and synaptojanin in vesicle formation (see, Ringstad N. et al., 1999, Neuron 24(1):143-154).

The human Sac domain-containing inositol phosphatase (hSac2) is ubiquitously expressed, but especially abundant in the brain, heart, skeletal muscle, and kidney. hSac2 protein exhibits 5-phosphatase activity specific for phosphatidylinositol 4,5-bisphosphate and phosphatidylinositol 3,4,5-trisphosphate (Minagawa T. et al., (2001) J Biol Chem 276(25):22011-22015).

Energy transduction in mitochondria requires the transport of many specific metabolites across the inner membrane of this eukaryotic organelle. The mitochondrial carrier family (MCF) consists of at least thirty-seven proteins. (Kuan J. and Saier M. H., 1993, Crit Rev Biochem Mol Biol 28(3):209-233). The mitochondrial aspartate/glutamate carrier catalyzes an important step in both the urea cycle and the aspartate/malate NADH shuttle. Citrin and aralar1 are homologous proteins belonging to the mitochondrial carrier family with EF-hand Ca²⁺ binding motifs in their N-terminal domains. Citrin and aralar1 are isoform Ca²⁺ stimulated aspartate/glutamate transporters in mitochondria (Palmieri L. et al., 2001, EMBO J 20(18):5060-9). Solute carrier family 25, member 13 (SLC25A13) encodes a calcium-binding mitochondrial carrier protein, designated citrin. Mutations in the SLC25A13 gene lead to adult-onset type II citrullinemia (Yasuda T. et al., 2000, Hum Genet 107(6):537-545).

The held out wings (how) Drosophila gene encodes a RNA-binding protein involved in the control of muscular and cardiac activity. The how protein is localized to the nucleus. how is highly related to the mouse quaking gene which plays a role at least in myelination and that could serve to link a signal transduction pathway to the control of mRNA metabolism (Zaffran S. et al., 1997, Development 124(10):2087-2098). Two isoforms of the Drosophila RNA binding protein, how, act in opposing directions to regulate tendon cell differentiation (Nabel-Rosen H. et al., 2002, Dev Cell February 2002;2(2):183-193). The opposing activities of the How isoforms are manifested by differential rates of mRNA degradation of the target stripe mRNA. This mechanism is conserved, as the mammalian RNA binding Quaking proteins may similarly affect the levels of Krox20, a regulator of Schwann cell maturation.

The mouse quaking (qk) gene is essential in both myelination and early embryogenesis. Its product, QKI, is an RNA-binding protein belonging to a growing protein family called STAR (signal transduction and activator of RNA) (Wu J. et al., 1999, J Biol Chem 274(41):29202-29210). Quaking is essential for blood vessel development (Noveroske J. K. et al., 2002, Genesis 32(3):218-230).

The myelin basic protein (MBP) gene is expressed in oligodendrocytes and Schwann cells, and expression follows a tightly regulated developmental time course. Cell type- and developmental stage-specific expression of the MBP gene is regulated by a series of cis-acting elements located upstream of the transcription start site. Myelin gene expression factor-2 (Myef-2), a protein isolated from mouse brain represses transcription of the MBP gene. Myef-2 mRNA is developmentally regulated in mouse brain; its peak expression occurs at postnatal day 7, prior to the onset of MBP expression (Haas S. et al., 1995, J Biol Chem 270(21):12503-12510).

MBP is a major component of the myelin sheath whose production is developmentally controlled during myelinogenesis. Programmed expression of the MBP gene is regulated at the level of transcription. The MB1 regulatory motif plays an important role in transcription of the MBP promoter. The MB1 element contains a binding site for the repressor protein MyEF-2 (Myelin gene expression factor-2). MyEF-2 is involved in transcriptional regulation of the MBP gene during the course of brain development (Muralidharan V. et al., 1997, J Cell Biochem Sep. 15, 1997;66(4):524-31).

The Drosophila melanogaster gene couch potato (cpo, GadFly Accession Number CG18434) encodes a putative nuclear RNA binding protein. The protein is expressed in the Drosophila embryo (embryonic central nervous system, embryonic peripheral nervous system, embryonic/larval midgut, glial cell and other tissues) (Harvie et al., 1998, Genetics 149(1): 217-231). At least three protein isoforms (for example, Cpo 17, Cpo 61.1 and Cpo. 61.2) and 49 recorded mutant alleles have been described. Mutations have been isolated which affect the larval ventral ganglion and are recessive lethal in Drosophila. Mutant cpo flies exhibit an abnormal and hypoactive behavior (Bellen et al., 1992, Genetics 131: 365-375, and Bellen et al., 1992, Genes Dev. 6: 2125-2136). This invention describes as human homolog proteins to the Drosophila cpo encoded gene product the RNA-binding protein gene with multiple splicing and a hypothetical protein XP_{—}091097. No further information is available for the human homolog proteins from the prior art.

Incomplete reduction of atmospheric oxygen generates potent oxidizing agents, including reactive oxygen species (ROS) and their toxic byproducts. Protection from ROS is mediated by nonenzymatic agents, enzymes, and low molecular weight reducing agents, such as thioredoxin. Under normal conditions, thioredoxin reductase reduces oxidized thioredoxin in the presence of NADPH. Reduced thioredoxin serves as an electron donor for thioredoxin peroxidase (peroxiredoxin) which consequently reduces H₂O₂ to H₂O (Schallreuter K. U. and Wood J. M., 2001, J Photochem Photobiol B 64(2-3):179-184). Members of the peroxiredoxin family play an antioxidant protective role in various tissues under nonpathologic conditions and during inflammatory processes. Antioxidants govern intracellular reduction-oxidation (redox) status, which plays a critical role in NFKB (nuclear factror kappa-B) transcription factor activation. Different antioxidants are selective for redox regulation of certain transcription factors. Peroxidases of the peroxiredoxin family reduce hydrogen peroxide H₂O₂ and alkyl hydroperoxides to water and alcohol with the use of reducing equivalents derived from thiol-containing donor molecules.

A family of highly conserved antioxidant enzymes, Peroxiredoxins (Prxs), has two major Prx subfamilies: one subfamily uses two conserved cysteines (2-Cys) and the other uses 1-Cys to scavenge reactive oxygen species (ROS). Four mammalian 2-Cys members (Prx I-IV) utilize thioredoxin as the electron donor for antioxidation. Prxs are capable of protecting cells from ROS insult and regulating the signal transduction pathways that utilize c-AbI, caspases, nuclear factor-kappaB (NF-kappaB) and activator protein-1 (AP-1) to influence cell growth and apoptosis. Prxs are also essential for red blood cell (RBC) differentiation and are capable of inhibiting human immunodeficiency virus (HIV) infection and organ transplant rejection (Butterfield L. H. et al., 1999, Antioxid Redox Signal 1(4):385-402). Distribution patterns indicate that Prxs are highly expressed in the tissues and cells at risk for diseases related to ROS toxicity, such as Alzheimer's and Parkinson's diseases and atherosclerosis. This correlation suggests that Prxs are protective against ROS toxicity, yet overwhelmed by oxidative stress in some cells (Butterfield L. H. et al., 1999, Antioxid Redox Signal 1(4):385-402). Prxs tend to form large aggregates at high concentrations, a feature that may interfere with their normal protective function or may even render them cytotoxic. Imbalance in the expression of subtypes can also potentially increase their susceptibility to oxidative stress. Therefor Prxs may play a role in the cellular dysfunction of ROS-related diseases ranging from atherosclerosis to cancer to neurodegenerative diseases.

The Drosophila gene with GadFly Accession Number CG14440 encodes for a protein which is most homologous to the human hypothetical protein LOC55565 (GenBank Accession Number NP_{—}060000.1 for the protein, NM_{—}017530 for the cDNA). No functional data are available for these proteins in the prior art.

So far, it has not been described that a protein of the invention or a homologous protein is involved in the regulation of energy homeostasis and body-weight regulation and related disorders, and thus, no functions in metabolic diseases and other diseases as listed above have been discussed. In this invention we demonstrate that the correct gene dose of a protein of the invention is essential for maintenance of energy homeostasis. A genetic screen was used to identify that mutation of a gene encoding a protein of the invention or a homologous gene causes changes in the metabolism, in particular related to obesity, which is reflected by a significant change of triglyceride content, the major energy storage substance.

Before the present proteins, nucleotide sequences, and methods are described, it is understood that this invention is not limited to the particular methodology, protocols, cell lines, vectors, and reagents described as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies that are reported in the publications which might be used in connection with the invention. Nothing herein is to be construed as an admission that the invention is not entitled to antedate such disclosure.

The present invention discloses that CG7956, aralar1, how, CG9373, cpo, Jafrac1, or CG14440 homologous proteins (herein referred to as “proteins of the invention” or “a protein of the invention”) are regulating the energy homeostasis and fat metabolism especially the metabolism and storage of triglycerides, and polynucleotides, which identify and encode the proteins disclosed in this invention. The invention also relates to vectors, host cells, antibodies, and recombinant methods for producing the polypeptides and polynucleotides of the invention. The invention also relates to the use of these sequences in the diagnosis, study, prevention, and treatment of metabolic diseases and dysfunctions, including metabolic syndrome, obesity, or diabetes as well as related disorders such as eating disorder, cachexia, hypertension, coronary heart disease, hypercholesterolemia, dyslipidemia, osteoarthritis, or gallstones.

GadFly Accession Number CG7956, aralar1 (GadFly Accession Number CG2139), how (GadFly Accession Number CG10293), GadFly Accession Number CG9373, cpo (GadFly Accession Number CG31243 and CG18434), Jafrac1 (GadFly Accession Number CG1633), or GadFly Accession Number CG14440 homologous proteins and nucleic acid molecules coding therefore are obtainable from insect or vertebrate species, e.g. mammals or birds. Particularly preferred are homologous nucleic acids, particularly nucleic acids encoding a human protein as described in TABLE 1.

The invention particularly relates to a nucleic acid molecule encoding a polypeptide contributing to regulating the energy homeostasis and the metabolism of triglycerides, wherein said nucleic acid molecule comprises
(a) the nucleotide sequence of CG7956, aralar1, how, CG9373, cpo, Jafrac1, or CG14440 or homologous nucleic acids, particularly nucleic acids encoding a human protein as described in Table 1, and/or a sequence complementary thereto,
(b) a nucleotide sequence which hybridizes at 50° C. in a solution containing 1×SSC and 0.1% SDS to a sequence of (a),
(c) a sequence corresponding to the sequences of (a) or (b) within the degeneration of the genetic code,
(d) a sequence which encodes a polypeptide which is at least 85%, preferably at least 90%, more preferably at least 95%, more preferably at least 98% and up to 99.6% identical to the amino acid sequences of CG7956, aralar1, how, CG9373, cpo, Jafrac1, or CG14440 homologous protein, preferably of a human homologous protein as described in Table 1.
(e) a sequence which differs from the nucleic acid molecule of (a) to (d) by mutation and wherein said mutation causes an alteration, deletion, duplication and/or premature stop in the encoded polypeptide or
(f) a partial sequence of any of the nucleotide sequences of (a) to (e) having a length of 15 bases, preferably 20 bases, more preferably 25 bases and most preferably at least 50 bases.

The invention is based on the finding that CG7956, aralar1, how, CG9373, cpo, Jafrac1, or CG14440 and/or homologous proteins and the polynucleotides encoding these, are involved in the regulation of triglyceride storage and therefore energy homeostasis. The invention describes the use of these compositions for the diagnosis, study, prevention, or treatment of metabolic diseases or dysfunctions, including metabolic syndrome, obesity, or diabetes, as well as related disorders such as eating disorder, cachexia, hypertension, coronary heart disease, hypercholesterolemia, dyslipidemia, osteoarthritis, or gallstones.

Accordingly, the present invention relates to genes with novel functions in body-weight regulation, energy homeostasis, metabolism, and obesity, functional fragments of said genes, polypeptides encoded by said genes or fragments thereof, and effectors/modulators thereof, e.g. antibodies, biologically active nucleic acids, such as antisense molecules, RNAi molecules or ribozymes, aptamers, peptides or low-molecular weight organic compounds recognizing said polynucleotides or polypeptides.

The ability to manipulate and screen the genomes of model organisms such as the fly Drosophila melanogaster provides a powerful tool to analyze biological and biochemical processes that have direct relevance to more complex vertebrate organisms due to significant evolutionary conservation of genes, cellular processes, and pathways (see, for example, Adams M. D. et al., (2000) Science 287: 2185-2195). Identification of novel gene functions in model organisms can directly contribute to the elucidation of correlative pathways in mammals (humans) and of methods of modulating them. A correlation between a pathology model (such as changes in triglyceride levels as indication for metabolic syndrome including obesity) and the modified expression of a fly gene can identify the association of the human ortholog with the particular human disease.

In one embodiment, a forward genetic screen is performed in fly displaying a mutant phenotype due to misexpression of a known gene (see, Johnston Nat Rev Genet 3: 176-188 (2002); Rorth P., (1996) Proc Natl Acad Sci U S A 93: 12418-12422). In this invention, we have used a genetic screen to identify mutations of the CG7956, aralar1, how, CG9373, cpo, Jafrac1, or CG14440 gene, or homologous genes that cause changes in the body weight, which are reflected by a significant change of triglyceride levels.

Obese people mainly show a significant increase in the content of triglycerides. Triglycerides are the most efficient storage for energy in cells. In order to isolate genes with a function in energy homeostasis, several thousand proprietary and publicly available EP-lines were tested for their triglyceride content after a prolonged feeding period (see Examples for more detail). Lines with significantly changed triglyceride content were selected as positive candidates for further analysis. The increase or decrease of triglyceride content due to the loss of a gene function suggests gene activities in energy homeostasis in a dose dependent manner that controls the amount of energy stored as triglycerides.

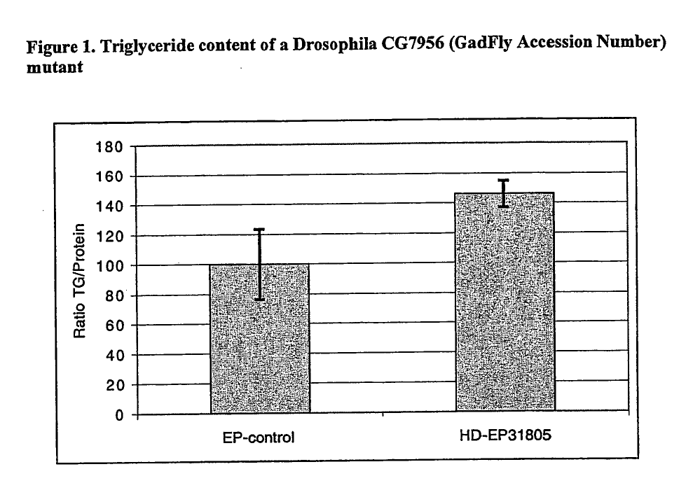

In this invention, the content of triglycerides of a pool of flies with the same genotype was analyzed after prolonged feeding using a triglyceride assay. Male flies homozygous or heterozygous for the integration of vectors for Drosophila EP-lines were analyzed in assays measuring the triglyceride contents of these flies, illustrated in more detail in the Examples section. The results of the triglyceride content analysis are shown in FIGS. 1, 5, 9, 13, 17, 21, and 25, respectively.

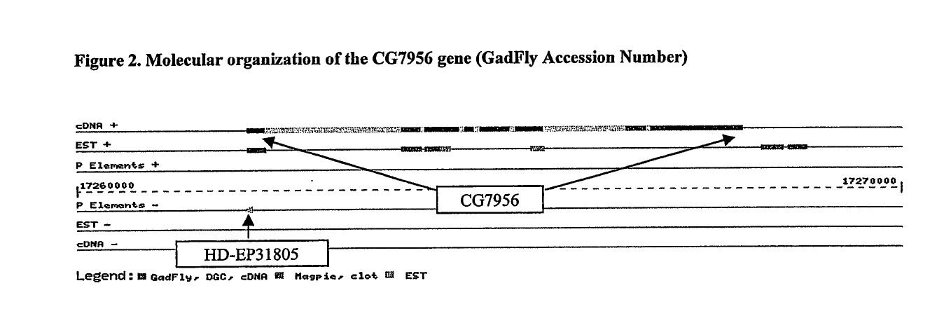

Genomic DNA sequences were isolated that are localized adjacent to the EP or PX vector integration. Using those isolated genomic sequences public databases like Berkeley Drosophila Genome Project (GadFly; see also FlyBase (1999) Nucleic Acids Research 27:85-88) were screened thereby identifying the integration sites of the vectors, and the corresponding genes, described in more detail in the Examples section. The molecular organization of the genes is shown in FIGS. 2, 6, 10, 14, 18, 22, and 26, respectively.

An additional screen using Drosophila mutants with modifications of the eye phenotype identified an interaction of cpo with adipose, a protein regulating, causing or contributing to obesity. An additional screen using Drosophila mutants with modifications of the eye phenotype identified a modification of UCP activity by cpo, thereby leading to an altered mitochondrial activity. These findings suggest the presence of similar activities of these described homologous proteins in humans that provides insight into diagnosis, treatment, and prognosis of metabolic disorders.

The Drosophila genes and proteins encoded thereby with functions in the regulation of triglyceride metabolism were further analysed in publicly available sequence databases (see Examples for more detail) and mammalian homologs were identified.

The function of the mammalian homologs in energy homeostasis was further validated in this invention by analyzing the expression of the transcripts in different tissues and by analyzing the role in adipocyte differentiation. Expression profiling studies (see Examples for more detail) confirm the particular relevance of the protein(s) of the invention as regulators of energy metabolism in mammals. Further, we show that the proteins of the invention are regulated by fasting and by genetically induced obesity. In this invention, we used mouse models of insulin resistance and/or diabetes, such as mice carrying gene knockouts in the leptin pathway (for example, ob (leptin) or db (leptin receptor) mice) to study the expression of the protein of the invention. Such mice develop typical symptoms of diabetes, show hepatic lipid accumulation and frequently have increased plasma lipid levels (see Bruning et al, 1998, Mol. Cell. 2:449-569).

Microarrays are analytical tools routinely used in bioanalysis. A microarray has molecules distributed over, and stably associated with, the surface of a solid support. The term “microarray” refers to an arrangement of a plurality of polynucleotides, polypeptides, antibodies, or other chemical compounds on a substrate. Microarrays of polypeptides, polynucleotides, and/or antibodies have been developed and find use in a variety of applications, such as monitoring gene expression, drug discovery, gene sequencing, gene mapping, bacterial identification, and combinatorial chemistry. One area in particular in which microarrays find use is in gene expression analysis (see Example 6). Array technology can be used to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for identifying genes that are tissue specific, are affected by a substance being tested in a toxicology assay, are part of a signaling cascade, carry out housekeeping functions, or are specifically related to a particular genetic predisposition, condition, disease, or disorder.

Microarrays may be prepared, used, and analyzed using methods known in the art (see for example, Brennan, T. M. et al. (1995) U.S. Pat. No. 5,474,796—Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:21502155; Heller, M. J. et al. (1997) U.S. Pat. No. 5,605,662). Various types of microarrays are well known and thoroughly described in Schena, M., ed. (1999; DNA Microarrays: A Practical Approach, Oxford University Press, London).

In further embodiments, oligonucleotides or longer fragments derived from any of the polynucleotides described herein may be used as elements on a microarray. The microarray can be used in transcript imaging techniques, which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents, which are highly effective and display the fewest side effects may be selected for a patient based on his/her pharmacogenomic profile.

As determined by Microarray analysis, Quaking 6 (QKI6), RNA binding protein HQK-7B, RNA binding protein with multiple splicing (RBPMS), Peroxiredoxin 1 (PRDX1), and hypothetical protein LOC55565 show differential expression in human primary adipocytes. Thus, Quaking 6 (QKI6), RNA binding protein HQK-7B, RNA binding protein with multiple splicing (RBPMS), Peroxiredoxin 1 (PRDX1), and hypothetical protein LOC55565 are strong candidates for the manufacture of a pharmaceutical composition and a medicament for the treatment of conditions related to human metabolism, such as obesity, diabetes, and/or metabolic syndrome.

The invention also encompasses polynucleotides that encode a protein of the invention or a homologous protein. Accordingly, any nucleic acid sequence, which encodes the amino acid sequences of a protein of the invention or a homologous protein, can be used to generate recombinant molecules that express a protein of the invention or a homologous protein. In a particular embodiment, the invention encompasses nucleic acids encoding Drosophila CG7956, aralar1, how, CG9373, cpo, Jafrac1, or CG14440 or human CG7956, aralar1, how, CG9373, cpo, Jafrac1, or CG14440 homologs; referred to herein as the proteins of the invention. It will be appreciated by those skilled in the art that as a result of the degeneracy of the genetic code, a multitude of nucleotide sequences encoding the proteins, some bearing minimal homology to the nucleotide sequences of any known and naturally occurring gene, may be produced. Thus, the invention contemplates each and every possible variation of nucleotide sequence that could be made by selecting combinations based on possible codon choices.

Also encompassed by the invention are polynucleotide sequences that are capable of hybridizing to the claimed nucleotide sequences, and in particular, those of the polynucleotides encoding CG7956, aralar1, how, CG9373, cpo, Jafrac1, or CG14440, or a homologous protein, preferably a human homologous protein as described in Table 1, under various conditions of stringency. Hybridization conditions are based on the melting temperature (Tm) of the nucleic acid binding complex or probe, as taught in Wahl, G. M. and S. L. Berger (1987: Methods Enzymol. 152:399-407) and Kimmel, A. R. (1987; Methods Enzymol. 152:507-511), and may be used at a defined stringency. Preferably, hybridization under stringent conditions means that after washing for 1 h with 1×SSC and 0.1% SDS at 50° C., preferably at 55° C., more preferably at 62° C. and most preferably at 68° C., particularly for 1 h in 0.2×SSC and 0.1% SDS at 50° C., preferably at 55° C., more preferably at 62° C. and most preferably at 68° C., a positive hybridization signal is observed. Altered nucleic acid sequences encoding the proteins which are encompassed by the invention include deletions, insertions, or substitutions of different nucleotides resulting in a polynucleotide that encodes the same or a functionally equivalent protein.

The encoded proteins may also contain deletions, insertions, or substitutions of amino acid residues, which produce a silent change and result in functionally equivalent proteins. Deliberate amino acid substitutions may be made on the basis of similarity in polarity, charge, solubility, hydrophobicity, hydrophilicity, and/or the amphipathic nature of the residues as long as the biological activity of the protein is retained. Furthermore, the invention relates to peptide fragments of the proteins or derivatives of such fragments such as cyclic peptides, retro-inverso peptides or peptide mimetics, wherein the peptides or derivatives usually have a length of at least four, preferably at least six and up to 50 amino acids.

Also included within the scope of the present invention are alleles of the genes encoding a protein of the invention or a homologous protein. As used herein, an “allele” or “allelic sequence” is an alternative form of the gene, which may result from at least one mutation in the nucleic acid sequence. Alleles may result in altered mRNAs or polypeptides whose structures or function may or may not be altered. Any given gene may have none, one, or many allelic forms. Common mutational changes, which give rise to alleles, are generally ascribed to natural deletions, additions, or substitutions of nucleotides. Each of these types of changes may occur alone, or in combination with the others, one or more times in a given sequence.

The nucleic acid sequences encoding a protein of the invention or a homologous protein may be extended utilizing a partial nucleotide sequence and employing various methods known in the art to detect upstream sequences such as promoters and regulatory elements. For example, one method which may be employed, “restriction-site” PCR, uses universal primers to retrieve unknown sequence adjacent to a known locus (Sarkar, G. (1993) PCR Methods Applic. 2:318-322). Inverse PCR may also be used to amplify or extend sequences using divergent primers based on a known region (Triglia, T. et al. (1988) Nucleic Acids Res. 16:8186). Another method which may be used is capture PCR which involves PCR amplification of DNA fragments adjacent to a known sequence in human and yeast artificial chromosome DNA (Lagerstrom, M. et al. (PCR Methods Applic. 1:111-119). Another method which may be used to retrieve unknown sequences is that of Parker, J. D. et al. (1991; Nucleic Acids Res. 19:3055-3060). Additionally, one may use PCR, nested primers, and PROMOTERFINDER libraries to walk in genomic DNA (Clontech, Palo Alto, Calif.). This process avoids the need to screen libraries and is useful in finding intron/exon junctions.

In order to express a biologically active protein, the nucleotide sequences encoding the proteins, may be inserted into appropriate expression vectors, i.e., a vector, which contains the necessary elements for the transcription and translation of the inserted coding sequence. Methods, which are well known to those skilled in the art, may be used to construct expression vectors containing sequences encoding the proteins and appropriate transcriptional and translational control elements. These methods include in vitro recombinant DNA techniques, synthetic techniques, and in vivo genetic recombination. Such techniques are described in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y.

In a further embodiment of the invention, nucleic acid sequences encoding the sequences of the invention may be ligated to a heterologous sequence to encode a fusion protein. Heterologous sequences are preferably located at the N-and/or C-terminus of the fusion protein.

A variety of expression vector/host systems may be utilized to contain and express sequences encoding the proteins. These include, but are not limited to, micro-organisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g. cauliflower mosaic virus, CaMV; tobacco mosaic virus, TMV) or with bacterial expression vectors (e.g., Ti or PBR322 plasmids); or animal, e.g. mammalian cell systems.

The presence of polynucleotide sequences encoding a protein of the invention or a homologous protein can be detected by DNA-DNA or DNA-RNA hybridization or amplification using probes or portions or fragments of polynucleotides encoding a protein of the invention or a homologous protein. Nucleic acid amplification based assays involve the use of oligonucleotides or oligomers based on the sequences specific for the gene to detect transformants containing DNA or RNA encoding the corresponding protein. As used herein “oligonucleotides” or “oligomers” refer to a nucleic acid sequence of at least about 10 nucleotides and as many as about 60 nucleotides, preferably about 15 to 30 nucleotides, and more preferably about 20-25 nucleotides, which can be used as a probe, primer or amplimer.

A variety of protocols for detecting and measuring the expression of proteins, using either polyclonal or monoclonal antibodies specific for the protein are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on the protein is preferred, but a competitive binding assay may be employed. These and other assays are described, among other places, in Hampton, R. et al. (1990; Serological Methods, a Laboratory Manual, APS Press, St Paul, Minn.) and Maddox, D. E. et al. (1983; J. Exp. Med. 158:1211-1216).

A wide variety of labels and conjugation techniques are known by those skilled in the art and may be used in various nucleic acid and proteins, e.g. immunological assays. Means for producing labeled hybridization or PCR probes for detecting sequences related to polynucleotides encoding a protein of the invention or a homologous protein include oligo-labeling, nick translation, end-labeling of RNA probes or PCR amplification using a labeled nucleotide. These procedures may be conducted using a variety of commercially available kits (Pharmacia & Upjohn, (Kalamazoo, Mich.); Promega (Madison Wis.); and U.S. Biochemical Corp., (Cleveland, Ohio).

Suitable reporter molecules or labels, which may be used for nucleic acid and protein assays, include radionuclides, enzymes, fluorescent, chemiluminescent, or chromogenic agents as well as substrates, co-factors, inhibitors, magnetic particles, and the like.

Host cells transformed with nucleotide sequences encoding the protein may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides which encode the protein may be designed to contain signal sequences, which direct secretion of the protein through a prokaryotic or eukaryotic cell membrane. Other recombinant constructions may be used to join sequences encoding the protein to nucleotide sequence encoding a polypeptide domain, which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAG extension/affinity purification system (Immunex Corp., Seattle, Wash.) The inclusion of cleavable linker sequences such as those specific for Factor XA or Enterokinase (Invitrogen, San Diego, Calif.) between the purification domain and the desired protein may be used to facilitate purification.

Diagnostics and Therapeutics

The data disclosed in this invention show that the nucleic acids and proteins of the invention and effectors/modulators thereof are useful in diagnostic and therapeutic applications implicated, for example but not limited to, in metabolic diseases or dysfunctions, including metabolic syndrome, obesity, or diabetes, as well as related disorders such as eating disorder, cachexia, hypertension, coronary heart disease, hypercholesterolemia, dyslipidemia, osteoarthritis, or gallstones. Hence, diagnostic and therapeutic uses for the nucleic acids and proteins of the invention are, for example but not limited to, the following: (i) protein therapy, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues).

The nucleic acids and proteins of the invention are useful in diagnostic and therapeutic applications implicated in various applications as described below. For example, but not limited to, cDNAs encoding the proteins of the invention and particularly their human homologues may be useful in gene therapy, and the proteins of the invention and particularly their human homologues may be useful when administered to a subject in need thereof. By way of non-limiting example, the compositions of the present invention will have efficacy for treatment of patients suffering from, for example, but not limited to, in metabolic disorders as described above.

The nucleic acid sequence encoding a protein of the invention, or a homologous protein, or a functional fragments thereof, may further be useful in diagnostic applications, wherein the presence or amount of the nucleic acids or the proteins are to be assessed. These materials are further useful in the generation of antibodies that bind immunospecifically to the novel substances of the invention for use in therapeutic or diagnostic methods.

For example, in one aspect, antibodies which are specific for a protein of the invention or a homologous protein may be used directly as an antagonist, or indirectly as a targeting or delivery mechanism for bringing a pharmaceutical agent to cells or tissue which express the protein. The antibodies may be generated using methods that are well known in the art. Such antibodies may include, but are not limited to, polyclonal, monoclonal, chimerical, single chain, Fab fragments, and fragments produced by a Fab expression library. Neutralising antibodies, (i.e., those which inhibit dimer formation) are especially preferred for therapeutic use.

For the production of antibodies, various hosts including goats, rabbits, rats, mice, humans, and others, may be immunized by injection with the protein or any fragment or oligopeptide thereof which has immunogenic properties. Depending on the host species, various adjuvants may be used to increase immunological response. It is preferred that the peptides, fragments, or oligopeptides used to induce antibodies to the protein have an amino acid sequence consisting of at least five amino acids, and more preferably at least 10 amino acids.

Monoclonal antibodies to the proteins may be prepared using any technique that provides for the production of antibody molecules by continuous cell lines in culture. These include, but are not limited to, the hybridoma technique, the human B-cell hybridoma technique, and the EBV-hybridoma technique (Köhler, G. et al. (1975) Nature 256:495-497; Kozbor, D. et al. (1985) J. Immunol. Methods 81:31-42; Cote, R. J. et al. Proc. Natl. Acad. Sci. 80:2026-2030; Cole, S. P. et al. (1984) Mol. Cell Biol. 62:109-120).

In addition, techniques developed for the production of “chimeric antibodies”, the splicing of mouse antibody genes to human antibody genes to obtain a molecule with appropriate antigen specificity and biological activity can be used (Morrison, S. L. et al. (1984) Proc. Natl. Acad. Sci. 81:6851-6855; Neuberger, M. S. et al (1984) Nature 312:604-608; Takeda, S. et al. (1985) Nature 314:452-454). Alternatively, techniques described for the production of single chain antibodies may be adapted, using methods known in the art, to produce single chain antibodies specific for a protein of the invention or a homologous protein. Antibodies with related specificity, but of distinct idiotypic composition, may be generated by chain shuffling from random combinatorial immunoglobulin libraries (Burton, D. R. (1991) Proc. Natl. Acad. Sci. 88:11120-3). Antibodies may also be produced by inducing in vivo production in the lymphocyte population or by screening recombinant immunoglobulin libraries or panels of highly specific binding reagents as disclosed in the literature (Orlandi, R. et al. (1989) Proc. Natl. Acad. Sci. 86:3833-3837; Winter, G. et al. (1991) Nature 349:293-299).

Antibody fragments which contain specific binding sites for the proteins may also be generated. For example, such fragments include, but are not limited to, the F(ab′)₂ fragments which can be produced by Pepsin digestion of the antibody molecule and the Fab fragments which can be generated by reducing the disulfide bridges of F(ab′)₂ fragments. Alternatively, Fab expression libraries may be constructed to allow rapid and easy identification of monoclonal Fab fragments with the desired specificity (Huse, W. D. et al. (1989) Science 254:1275-1281).

Various immunoassays may be used for screening to identify antibodies having the desired specificity. Numerous protocols for competitive binding and immunoradiometric assays using either polyclonal or monoclonal antibodies with established specificities are well known in the art. Such immunoassays typically involve the measurement of complex formation between the protein and its specific antibody. A two-site, monoclonal-based immunoassay utilising monoclonal antibodies reactive to two non-interfering protein epitopes are preferred, but a competitive binding assay may also be employed (Maddox, supra).

In another embodiment of the invention, the polynucleotides or fragments thereof, or nucleic acid effector molecules such as antisense molecules, aptamers, RNAi molecules or ribozymes may be used for therapeutic purposes. In one aspect, aptamers, i.e. nucleic acid molecules, which are capable of binding to a protein of the invention and modulating its activity may be generated by a screening and selection procedure involving the use of combinatorial nucleic acid libraries.

In a further aspect, antisense molecules may be used in situations in which it would be desirable to block the transcription of the mRNA. In particular, cells may be transformed with sequences complementary to polynucleotides encoding a protein of the invention or a homologous protein. Thus, antisense molecules may be used to modulate/effect protein activity, or to achieve regulation of gene function. Such technology is now well know in the art, and sense or antisense oligomers or larger fragments, can be designed from various locations along the coding or control regions of sequences encoding the proteins. Expression vectors derived from retroviruses, adenovirus, herpes or vaccinia viruses, or from various bacterial plasmids may be used for delivery of nucleotide sequences to the targeted organ, tissue or cell population. Methods, which are well known to those skilled in the art, can be used to construct recombinant vectors, which will express antisense molecules complementary to the polynucleotides of the genes encoding a protein of the invention or a homologous protein. These techniques are described both in Sambrook et al. (supra) and in Ausubel et al. (supra). Genes encoding a protein of the invention or a homologous protein can be turned off by transforming a cell or tissue with expression vectors which express high levels of polynucleotide which encodes a protein of the invention or a homologous protein or a functional fragment thereof. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector and even longer if appropriate replication elements are part of the vector system.

As mentioned above, modifications of gene expression can be obtained by designing antisense molecules, e.g. DNA, RNA, or nucleic acid analogues such as PNA, to the control regions of the genes encoding a protein of the invention or a homologous protein, i.e., the promoters, enhancers, and introns. Oligonucleotides derived from the transcription initiation site, e.g., between positions −10 and +10 from the start site, are preferred. Similarly, inhibition can be achieved using “triple helix” base-pairing methodology. Triple helix pairing is useful because it cause inhibition of the ability of the double helix to open sufficiently for the binding of polymerases, transcription factors, or regulatory molecules. Recent therapeutic advances using triplex DNA have been described in the literature (Gee, J. E. et al. (1994) In; Huber, B. E. and B. I. Carr, Molecular and Immunologic Approaches, Futura Publishing Co., Mt. Kisco, N.Y.). The antisense molecules may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. Examples, which may be used, include engineered hammerhead motif ribozyme molecules that can be specifically and efficiently catalyze endonucleolytic cleavage of sequences encoding a protein of the invention or a homologous protein. Specific ribozyme cleavage sites within any potential RNA target are initially identified by scanning the target molecule for ribozyme cleavage sites which include the following sequences: GUA, GUU, and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Nucleic acid effector molecules, e.g. antisense molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding a protein of the invention or a homologous protein. Such DNA sequences may be incorporated into a variety of vectors with suitable RNA polymerase promoters such as T7 or SP6. Alternatively, these cDNA constructs that synthesize antisense RNA constitutively or inducibly can be introduced into cell lines, cells, or tissues. RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of non-traditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection and by liposome injections may be achieved using methods, which are well known in the art. Any of the therapeutic methods described above may be applied to any suitable subject including, for example, mammals such as dogs, cats, cows, horses, rabbits, monkeys, and most preferably, humans.

An additional embodiment of the invention relates to the administration of a pharmaceutical composition, in conjunction with a pharmaceutically acceptable carrier, for any of the therapeutic effects discussed above. Such pharmaceutical compositions may consist of a protein of the invention or a homologous nucleic acid sequence or protein, antibodies to a protein of the invention or a homologous protein, mimetics, agonists, antagonists, or inhibitors of a protein of the invention or a homologous protein or nucleic acid sequence. The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone, or in combination with other agents, drugs or hormones. The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries, which facilitate processing of the active compounds into preparations which, can be used pharmaceutically. Further details on techniques for formulation and administration may be found in the latest edition of Remington's Pharmaceutical Sciences (Maack Publishing Co., Easton, Pa.).

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes. The pharmaceutical composition may be provided as a salt and can be formed with many acids. After pharmaceutical compositions have been prepared, they can be placed in an appropriate container and labeled for treatment of an indicated condition. For administration of proteins, such labeling would include amount, frequency, and method of administration.

Pharmaceutical compositions suitable for use in the invention include compositions wherein the active ingredients are contained in an effective amount to achieve the intended purpose. The determination of an effective dose is well within the capability of those skilled in the art. For any compounds, the therapeutically effective dose can be estimated initially either in cell culture assays, e.g., of preadipocyte cell lines, or in animal models, usually mice, rabbits, dogs, or pigs. The animal model may also be used to determine the appropriate concentration range and route of administration. Such information can then be used to determine useful doses and routes for administration in humans. A therapeutically effective dose refers to that amount of active ingredient, for example a protein of the invention or a homologous protein or nucleic acid sequence or functional fragment thereof, or antibodies, which is sufficient for treating a specific condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. Pharmaceutical compositions, which exhibit large therapeutic indices, are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage from employed, sensitivity of the patient, and the route of administration. The exact dosage will be determined by the practitioner, in light of factors related to the subject that requires treatment. Dosage and administration are adjusted to provide sufficient levels of the active moiety or to maintain the desired effect. Factors, which may be taken into account, include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation. Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art employ different formulations for nucleotides than for proteins or their inhibitors. Similarly, delivery of polynucleotides or polypeptides will be specific to particular cells, conditions, locations, etc.

In another embodiment, antibodies which specifically bind to a protein of the invention may be used for the diagnosis of conditions or diseases characterized by or associated with over- or underexpression of a protein of the invention or a homologous protein, or in assays to monitor patients being treated with a protein of the invention or a homologous protein, agonists, antagonists or inhibitors. The antibodies useful for diagnostic purposes may be prepared in the same manner as those described above for therapeutics. Diagnostic assays include methods which utilize the antibody and a label to detect the protein in human body fluids or extracts of cells or tissues. The antibodies may be used with or without modification, and may be labeled by joining them, either covalently or non-covalently, with a reporter molecule. A wide variety of reporter molecules which are known in the art may be used several of which are described above.

A variety of protocols including ELISA, RIA, and FACS for measuring proteins are known in the art and provide a basis for diagnosing altered or abnormal levels of gene expression. Normal or standard values for gene expression are established by combining body fluids or cell extracts taken from normal mammalian subjects, preferably human, with antibodies to the protein under conditions suitable for complex formation. The amount of standard complex formation may be quantified by various methods, but preferably by photometric means. Quantities of protein expressed in control and disease, samples e.g. from biopsied tissues are compared with the standard values. Deviation between standard and subject values establishes the parameters for diagnosing disease.

In another embodiment of the invention, the polynucleotides specific for a protein of the invention or a homologous protein may be used for diagnostic purposes. The polynucleotides, which may be used, include oligonucleotide sequences, antisense RNA and DNA molecules, and PNAs. The polynucleotides may be used to detect and quantitate gene expression in biopsied tissues in which gene expression may be correlated with disease. The diagnostic assay may be used to distinguish between absence, presence, and excess gene expression, and to monitor regulation of protein levels during therapeutic intervention.

In one aspect, hybridization with PCR probes which are capable of detecting polynucleotide sequences, including genomic sequences, encoding a protein of the invention or a homologous protein or closely related molecules, may be used to identify nucleic acid sequences which encode the respective protein. The hybridization probes of the subject invention may be DNA or RNA and are preferably derived from the nucleotide sequence of the polynucleotide encoding a CG7956, aralar1, how, CG9373, cpo, Jafrac1, or CG14440 homologous protein, preferably a human homologous protein as described in Table 1 or from a genomic sequence including promoter, enhancer elements, and introns of the naturally occurring gene. Means for producing specific hybridization probes for DNAs encoding a protein of the invention or a homologous protein include the cloning of nucleic acid sequences specific for a protein of the invention or a homologous protein into vectors for the production of mRNA probes. Such vectors are known in the art, commercially available, and may be used to synthesize RNA probes in vitro by means of the addition of the appropriate RNA polymerases and the appropriate labeled nucleotides. Hybridization probes may be labeled by a variety of reporter groups, for example, radionuclides such as ³²P or ³⁵S, or enzymatic labels, such as alkaline phosphatase coupled to the probe via avidin/biotin coupling systems, and the like.

Polynucleotide sequences specific for a protein of the invention or homologous nucleic acids may be used for the diagnosis of conditions or diseases, which are associated with the expression of the proteins. Examples of such conditions or diseases include, but are not limited to, metabolic diseases and disorders, including obesity and diabetes. Polynucleotide sequences specific for a protein of the invention or a homologous protein may also be used to monitor the progress of patients receiving treatment for metabolic diseases and disorders, including obesity and diabetes. The polynucleotide sequences may be used in Southern or Northern analysis, dot blot, or other membrane-based technologies; in PCR technologies; or in dip stick, pin, ELISA or chip assays utilizing fluids or tissues from patient biopsies to detect altered gene expression. Such qualitative or quantitative methods are well known in the art.

In a particular aspect, the nucleotide sequences specific for a protein of the invention or homologous nucleic acids may be useful in assays that detect activation or induction of various metabolic diseases or dysfunctions, including metabolic syndrome, obesity, or diabetes. The nucleotide sequences may be labeled by standard methods, and added to a fluid or tissue sample from a patient under conditions suitable for the formation of hybridization complexes. After a suitable incubation period, the sample is washed and the signal is quantitated and compared with a standard value. The presence of the associated disease. Such assays may also be used to evaluate the efficacy of a particular therapeutic treatment regimen in animal studies, in clinical trials, or in monitoring the treatment of an individual patient.

In order to provide a basis for the diagnosis of a disease associated with expression of a protein of the invention or a homologous protein, a normal or standard profile for expression is established. This may be accomplished by combining body fluids or cell extracts taken from normal subjects, either animal or human, with a sequence, or a fragment thereof, which is specific for nucleic acids encoding a protein of the invention or homologous nucleic acids, under conditions suitable for hybridization or amplification. Standard hybridization may be quantified by comparing the values obtained from normal subjects with those from an experiment where a known amount of a substantially purified polynucleotide is used. Standard values obtained from normal samples may be compared with values obtained from samples from patients who are symptomatic for disease. Deviation between standard and subject values is used to establish the presence of disease. Once disease is established and a treatment protocol is initiated, hybridization assays may be repeated on a regular basis to evaluate whether the level of expression in the patient begins to approximate that, which is observed in the normal patient. The results obtained from successive assays may be used to show the efficacy of treatment over a period ranging from several days to months.

With respect to metabolic diseases or dysfunctions, including metabolic syndrome, obesity, or diabetes, the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the metabolic diseases and disorders. Additional diagnostic uses for oligonucleotides designed from the sequences encoding a protein of the invention or a homologous protein may involve the use of PCR. Such oligomers may be chemically synthesized, generated enzymatically, or produced from a recombinant source. Oligomers will preferably consist of two nucleotide sequences, one with sense orientation (5prime.fwdarw.3prime) and another with antisense (3prime.rarw.5prime), employed under optimized conditions for identification of a specific gene or condition. The same two oligomers, nested sets of oligomers, or even a degenerate pool of oligomers may be employed under less stringent conditions for detection and/or quantification of closely related DNA or RNA sequences.

Methods which may also be used to quantitate the expression of a protein of the invention or a homologous protein include radiolabeling or biotinylating nucleotides, coamplification of a control nucleic acid, and standard curves onto which the experimental results are interpolated (Melby, P. C. et al. (1993) J. Immunol. Methods, 159:235-244; Duplaa, C. et al. (1993) Anal. Biochem. 212:229-236). The speed of quantification of multiple samples may be accelerated by running the assay in an ELISA format where the oligomer of interest is presented in various dilutions and a spectrophotometric or calorimetric response gives rapid quantification.

In another embodiment of the invention, the nucleic acid sequences which are specific for a protein of the invention or homologous nucleic acids may also be used to generate hybridization probes, which are useful for mapping the naturally occurring genomic sequence. The sequences may be mapped to a particular chromosome or to a specific region of the chromosome using well known techniques. Such techniques include FISH, FACS, or artificial chromosome constructions, such as yeast artificial chromosomes, bacterial artificial chromosomes, bacterial P1 constructions or single chromosome cDNA libraries as reviewed in Price, C. M. (1993) Blood Rev. 7:127-134, and Trask, B. J. (1991) Trends Genet. 7:149-154. FISH (as described in Verma et al. (1988) Human Chromosomes: A Manual of Basic Techniques, Pergamon Press, New York, N.Y.) may be correlated with other physical chromosome mapping techniques and genetic map data. Examples of genetic map data can be found in the 1994 Genome Issue of Science (265:1981f). Correlation between the location of the gene encoding a protein of the invention or a homologous protein on a physical chromosomal map and a specific disease, or predisposition to a specific disease, may help to delimit the region of DNA associated with that genetic disease.

The nucleotide sequences of the subject invention may be used to detect differences in gene sequences between normal, carrier, or affected individuals. An analysis of polymorphisms, e.g. single nucleotide polymorphisms may be carried out. Further, in situ hybridization of chromosomal preparations and physical mapping techniques such as linkage analysis using established chromosomal markers may be used for extending genetic maps. Often the placement of a gene on the chromosome of another mammalian species, such as mouse, may reveal associated markers even if the number or arm of a particular human chromosome is not known. New sequences can be assigned to chromosomal arms, or parts thereof, by physical mapping. This provides valuable information to investigators searching for disease genes using positional cloning or other gene discovery techniques. Once the disease or syndrome has been crudely localized by genetic linkage to a particular genomic region, for example, AT to 11q22-23 (Gatti, R. A. et al. (1988) Nature 336:577-580), any sequences mapping to that area may represent associated or regulatory genes for further investigation. The nucleotide sequences of the subject invention may also be used to detect differences in the chromosomal location due to translocation, inversion, etc. among normal, carrier, or affected individuals.

In another embodiment of the invention, a protein of the invention or a homologous protein, its catalytic or immunogenic fragments or oligopeptides thereof, an in vitro model, a genetically altered cell or animal, can be used for screening libraries of compounds, e.g. peptides or low-molecular weight organic compounds, in any of a variety of drug screening techniques. One can identify modulators/effectors, e.g. receptors, enzymes, proteins, ligands, or substrates that bind to, modulate or mimic the action of one or more of the proteins of the invention. The protein or fragment employed in such screening may be free in solution, affixed to a solid support, borne on a cell surface, or located intracellularly. The formation of binding complexes, between a protein of the invention or a homologous protein and the agent tested, may be measured. Agents may also, either directly or indirectly, influence the activity of the proteins of the invention.

In addition activity of the proteins of the invention against their physiological substrate(s) or derivatives thereof could be measured in cell-based assays. Agents may also interfere with posttranslational modifications of the proteins of the invention, such as phosphorylation and dephosphorylation, farnesylation, palmitoylation, acetylation, alkylation, ubiquitination, proteolytic processing, subcellular localization and degradation. Moreover, agents could influence the dimerization or oligomerization of the proteins of the invention or, in a heterologous manner, of the proteins of the invention with other proteins, for example, but not exclusively, docking proteins, enzymes, receptors, ion channels, uncoupling proteins, or translation factors. Agents could also act on the physical interaction of the proteins of this invention with other proteins, which are required for protein function, for example, but not exclusively, their downstream signaling.

The phosphatase activity of the Sac domain-containing inositol phosphatase 2 (SAC2) of the invention could be measured in vitro by using recombinantly expressed and purified SAC2 or fragments thereof by making use of artificial phosphatase substrates well known in the art, i.e. but not exclusively DiFMUP or FDP (Molecular Probes, Eugene, Oreg.), which are converted to fluorophores or chromophores upon dephosphorylation. Alternatively, the dephosphorylation of physiological substrates of SAC2 could be measured by making use of any of the well known screening technologies suitable for the detection of the phosphorylation status of SAC2 inositol substrates, i.e. in a procedure similar as described for the inositol phosphatase SHIP2 (T. Habib et al. (1998), JBC 273, 18605-18609). In addition activity of SAC2 against its physiological substrate(s) or derivatives thereof could be measured in cell-based assays, thereby determining activity of the phosphatase at the level of their downstream signalling.

Methods for determining protein-protein interaction are well known in the art. For example binding of a fluorescently labeled peptide derived from a protein of the invention to the interacting protein (or vice versa) could be detected by a change in polarisation. In case that both binding partners, which can be either the full length proteins as well as one binding partner as the full length protein and the other just represented as a peptide are fluorescently labeled, binding could be detected by fluorescence energy transfer (FRET) from one fluorophore to the other. In addition, a variety of commercially available assay principles suitable for detection of protein-protein interaction are well known In the art, for example but not exclusively AlphaScreen (PerkinElmer) or Scintillation Proximity Assays (SPA) by Amersham. Alternatively, the interaction of the proteins of the invention with cellular proteins could be the basis for a cell-based screening assay, in which both proteins are fluorescently labeled and interaction of both proteins is detected by analysing cotranslocation of both proteins with a cellular imaging reader, as has been developed for example, but not exclusively, by Cellomics or EvotecOAI. In all cases the two or more binding partners can be different proteins with one being the protein of the invention, or in case of dimerization and/or oligomerization the protein of the invention itself. Proteins of the invention, for which one target mechanism of interest, but not the only one, would be such protein/protein interactions are CG7956, aralar1, how, CG9373, cpo, Jafrac1, or CG14440 homologous proteins.

Assays for determining enzymatic and carrier activity of the proteins of the invention are well known in the art. Well known in the art are also a variety of assay formats to measure receptor-ligand binding.

Of particular interest are screening assays for agents that have a low toxicity for mammalian cells. The term “agent” as used herein describes any molecule, e.g. protein or pharmaceutical, with the capability of altering or mimicking the physiological function of one or more of the proteins of the invention. Candidate agents encompass numerous chemical classes, though typically they are organic molecules, preferably small organic compounds having a molecular weight of more than 50 and less than about 2,500 Daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise carbocyclic or heterocyclic structures and/or aromatic or polyaromatic structures substituted with one or more of the above functional groups.

Candidate agents are also found among biomolecules including peptides, saccharides, fatty acids, steroids, purines, pyrimidines, nucleic acids and derivatives, structural analogs or combinations thereof. Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Where the screening assay is a binding assay, one or more of the molecules may be joined to a label, where the label can directly or indirectly provide a detectable signal.

Another technique for drug screening, which may be used, provides for high throughput screening of compounds having suitable binding affinity to the protein of interest as described in published PCT application WO84/03564. In this method, as applied to a protein of the invention or a homologous protein, large numbers of different small test compounds are synthesized on a solid substrate, such as plastic pins or some other surface. The test compounds are reacted with the proteins, or fragments thereof, and washed. Bound proteins are then detected by methods well known in the art. Purified proteins can also be coated directly onto plates for use in the aforementioned drug screening techniques. Alternatively, non-neutralizing antibodies can be used to capture the peptide and immobilize it on a solid support. In another embodiment, one may use competitive drug screening assays in which neutralizing antibodies capable of binding a protein of the invention specifically compete with a test compound for binding the protein. In this manner, the antibodies can be used to detect the presence of any peptide, which shares one or more antigenic determinants with the protein of the invention.

The nucleic acids encoding the proteins of the invention can be used to generate transgenic cell lines and animals. These transgenic non-human animals are useful in the study of the function and regulation of the proteins of the invention in vivo. Transgenic animals, particularly mammalian transgenic animals, can serve as a model system for the investigation of many developmental and cellular processes common to humans. A variety of non-human models of metabolic disorders can be used to test modulators of the protein of the invention. Misexpression (for example, overexpression or lack of expression) of the protein of the invention, particular feeding conditions, and/or administration of biologically active compounts can create models of metablic disorders.

In one embodiment of the invention, such assays use mouse models of insulin resistance and/or diabetes, such as mice carrying gene knockouts in the leptin pathway (for example, ob (leptin) or db (leptin receptor) mice). Such mice develop typical symptoms of diabetes, show hepatic lipid accumulation and frequently have increased plasma lipid levels (see Bruning et al, 1998, Mol. Cell. 2:449-569). Susceptible wild type mice (for example C57BI/6) show similiar symptoms if fed a high fat diet. In addition to testing the expression of the proteins of the invention in such mouse strains (see EXAMPLES section), these mice could be used to test whether administration of a candidate modulator alters for example lipid accumulation in the liver, in plasma, or adipose tissues using standard assays well known in the art, such as FPLC, calorimetric assays, blood glucose level tests, insulin tolerance tests and others.

Transgenic animals may be made through homologous recombination in non-human embryonic stem cells, where the normal locus of the gene encoding the protein of the invention is mutated. Alternatively, a nucleic acid construct encoding the protein is injected into oocytes and is randomly integrated into the genome. One may also express the genes of the invention or variants thereof in tissues where they are not normally expressed or at abnormal times of development. Furthermore, variants of the genes of the invention like specific constructs expressing anti-sense molecules or expression of dominant negative mutations, which will block or alter the expression of the proteins of the invention may be randomly integrated into the genome. A detectable marker, such as lac Z or luciferase may be introduced into the locus of the genes of the invention, where upregulation of expression of the genes of the invention will result in an easily detectable change in phenotype. Vectors for stable integration include plasmids, retroviruses and other animal viruses, yeast artificial chromosomes (YACs), and the like.

DNA constructs for homologous recombination will contain at least portions of the genes of the invention with the desired genetic modification, and will include regions of homology to the target locus. Conveniently, markers for positive and negative selection are included. DNA constructs for random integration do not need to contain regions of homology to mediate recombination. DNA constructs for random integration will consist of the nucleic acids encoding the proteins of the invention, a regulatory element (promoter), an intron and a poly-adenylation signal. Methods for generating cells having targeted gene modifications through homologous recombination are known in the field. For non-human embryonic stem (ES) cells, an ES cell line may be employed, or embryonic cells may be obtained freshly from a host, e.g. mouse, rat, guinea pig, etc. Such cells are grown on an appropriate fibroblast-feeder layer and are grown in the presence of leukemia inhibiting factor (LIF).

When non-human ES or non-human embryonic cells or somatic pluripotent stem cells have been transformed, they may be used to produce transgenic animals. After transformation, the cells are plated onto a feeder layer in an appropriate medium. Cells containing the construct may be selected by employing a selective medium. After sufficient time for colonies to grow, they are picked and analyzed for the occurrence of homologous recombination or integration of the construct. Those colonies that are positive may then be used for embryo transfection and blastocyst injection. Blastocysts are obtained from 4 to 6 week old superovulated females. The ES cells are trypsinized, and the modified cells are injected into the blastocoel of the blastocyst. After injection, the blastocysts are returned to each uterine horn of pseudopregnant females. Females are then allowed to go to term and the resulting offspring is screened for the construct. By providing for a different phenotype of the blastocyst and the genetically modified cells, chimeric progeny can be readily detected. The chimeric animals are screened for the presence of the modified gene and males and females having the modification are mated to produce homozygous progeny. If the gene alterations cause lethality at some point in development, tissues or organs can be maintained as allogenic or congenic grafts or transplants, or in vitro culture. The transgenic animals may be any non-human mammal, such as laboratory animal, domestic animals, etc. The transgenic animals may be used in functional studies, drug screening, etc.

Finally, the invention also relates to a kit comprising at least one of
(a) a CG7956, aralar1, how, CG9373, cpo, Jafrac1, or CG14440 homologous nucleic acid molecule or a functional fragment thereof;
(b) a CG7956, aralar1, how, CG9373, cpo, Jafrac1, or CG14440 homologous amino acid molecule or a functional fragment or an isoform thereof;
(c) a vector comprising the nucleic acid of (a);
(d) a host cell comprising the nucleic acid of (a) or the vector of (c);
(e) a polypeptide encoded by the nucleic acid of (a);
(f) a fusion polypeptide encoded by the nucleic acid of (a);
(g) an antibody, an aptamer or another effector/modulator against the nucleic acid of (a) or the polypeptide of (b), (e), or (f) and
(h) an anti-sense oligonucleotide of the nucleic acid of (a).

The kit may be used for diagnostic or therapeutic purposes or for screening applications as described above. The kit may further contain user instructions.

The Figures show:

FIG. 1 shows the triglyceride content of a Drosophila Gadfly Accession Number CG7956 mutant. Shown is the change of triglyceride content of HD-EP(3)31805 flies caused by integration of the P-vector 3 base pairs 5′ of the CG7956 transcription unit (referred to as ‘HD-EP31805’, column 2) in comparison to controls containing all flies of the EP collection (referred to as ‘EP-control’, column 1).

FIG. 2 shows the molecular organization of the mutated CG7956 (Gadfly Accession Number) gene locus.

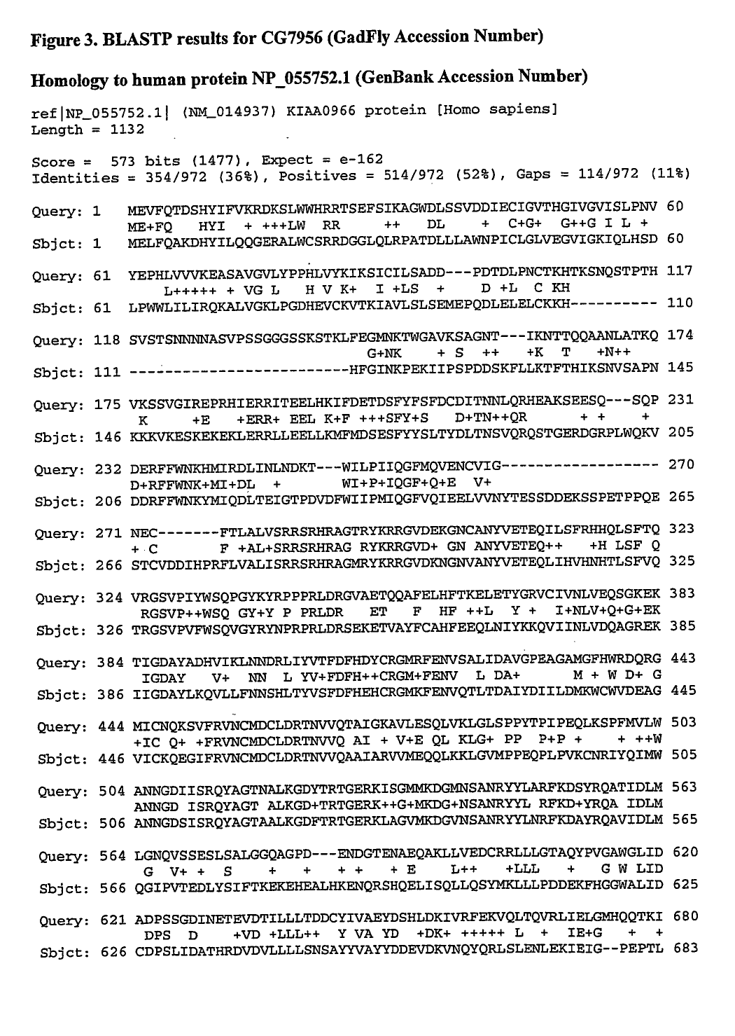

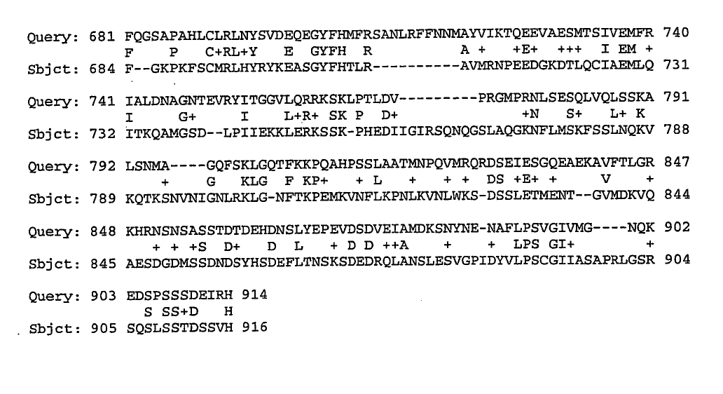

FIG. 3 shows the BLASTP search result for the Gadfly Accession Number CG7956 gene product (Query) with the best human homologous match (Sbjct).

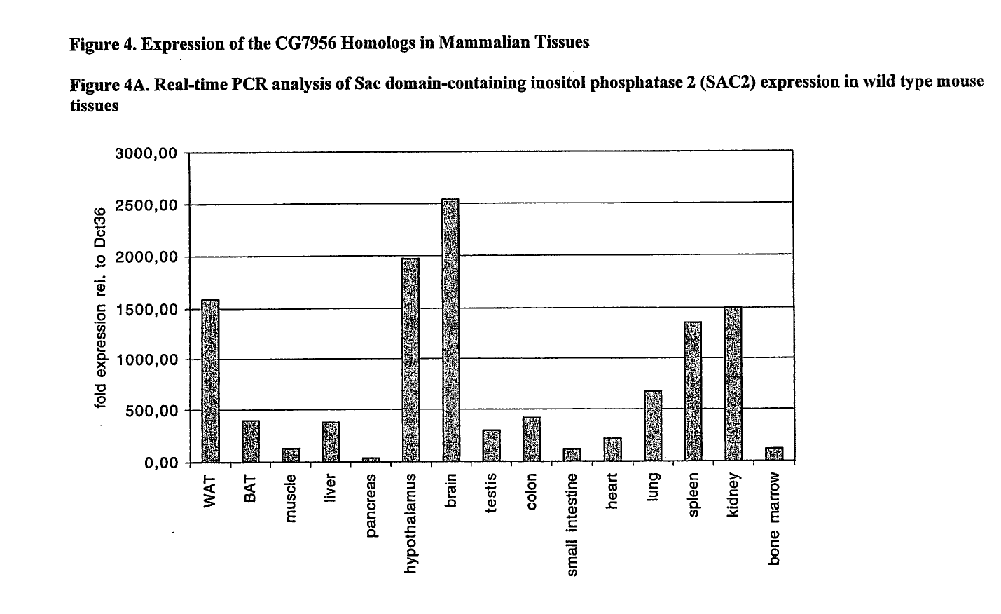

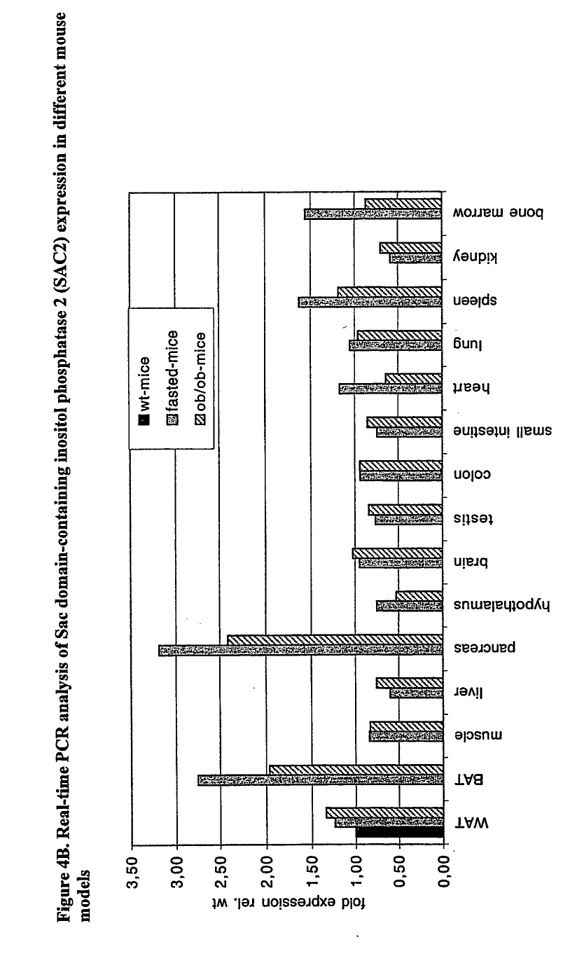

FIG. 4 shows the expression of the CG7956 homolog in mammalian tissues.

FIG. 4A shows the real-time PCR analysis of Sac domain-containing inositol phosphatase 2 (SAC2) expression in wild-type mouse tissues.

FIG. 4B shows the real-time PCR analysis of SAC2 expression in different mouse models.

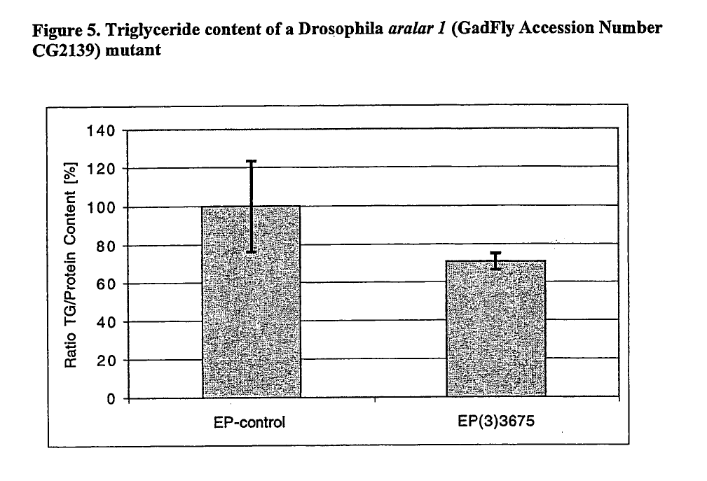

FIG. 5 shows the triglyceride content of a Drosophila aralar 1 (Gadfly Accession Number CG2139) mutant. Shown is the change of triglyceride content of EP(3)3675 flies caused by integration of the P-vector into an intron of the CG2139 gene (referred to as ‘EP(3)3675’, column 2) in comparison to controls containing all flies of the EP collection (referred to as ‘EP-control’, column 1).

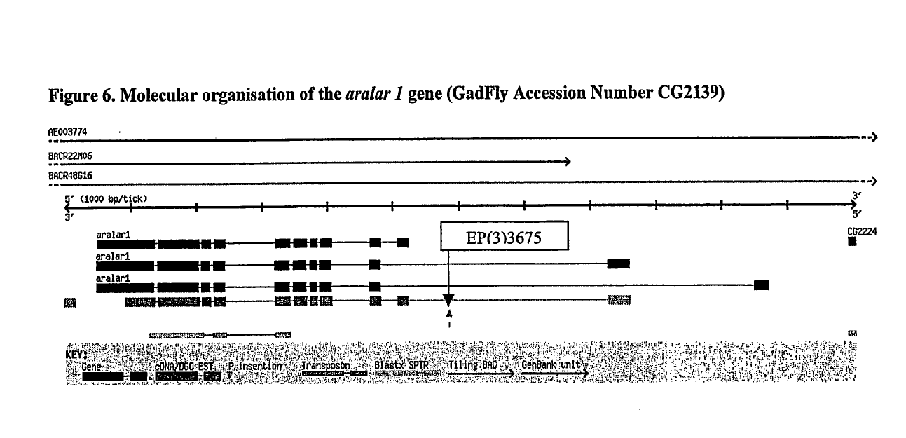

FIG. 6 shows the molecular organization of the mutated aralar 1 (Gadfly Accession Number CG2139) gene locus.

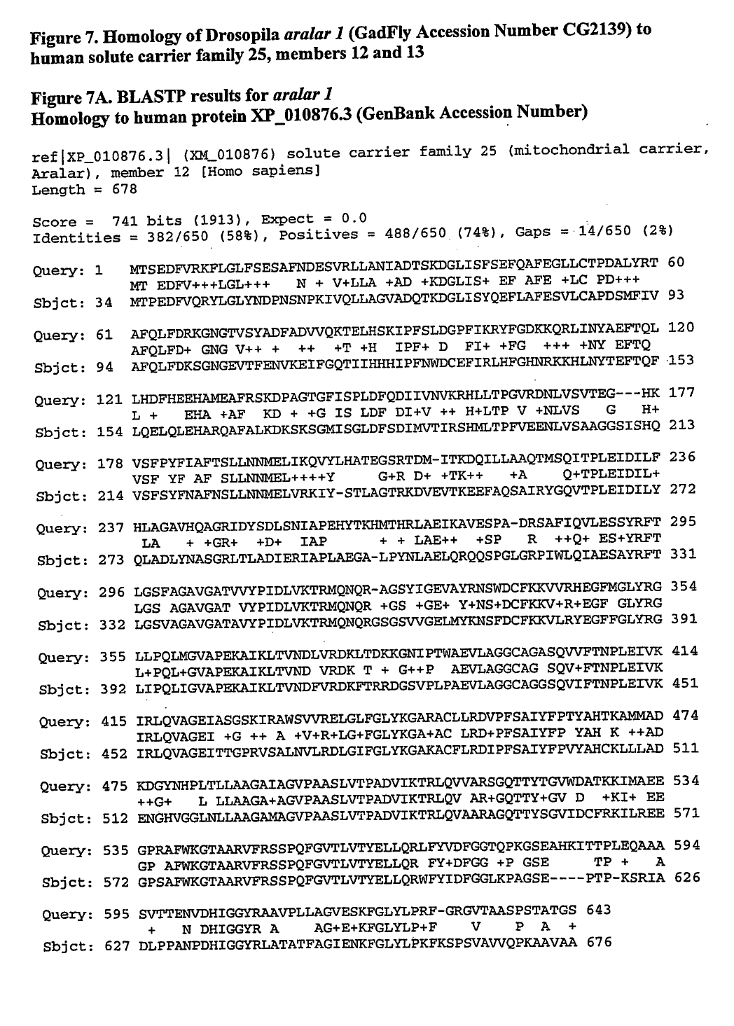

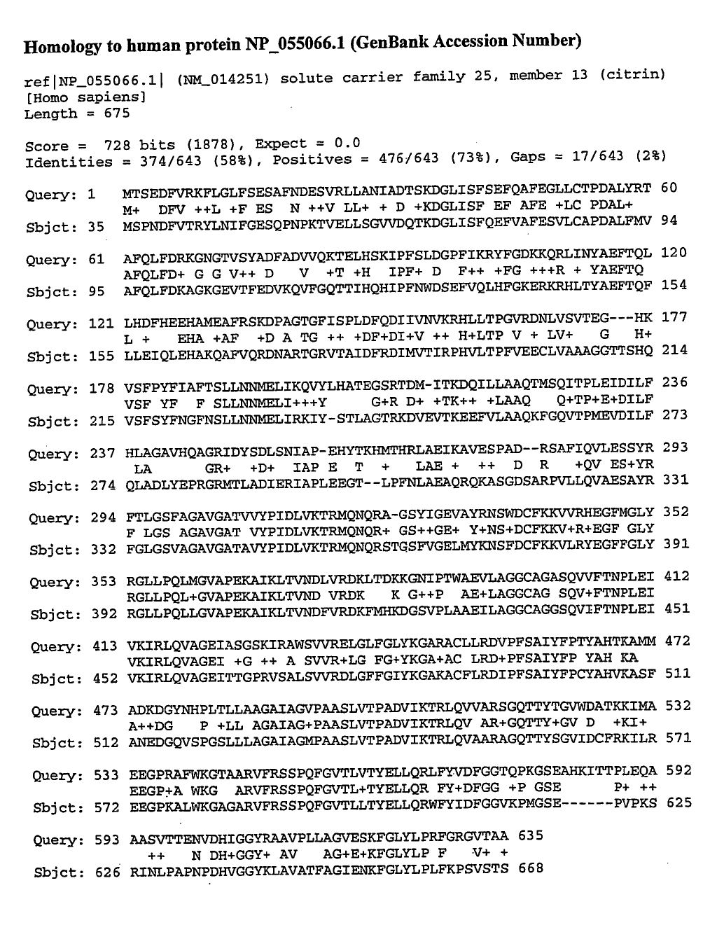

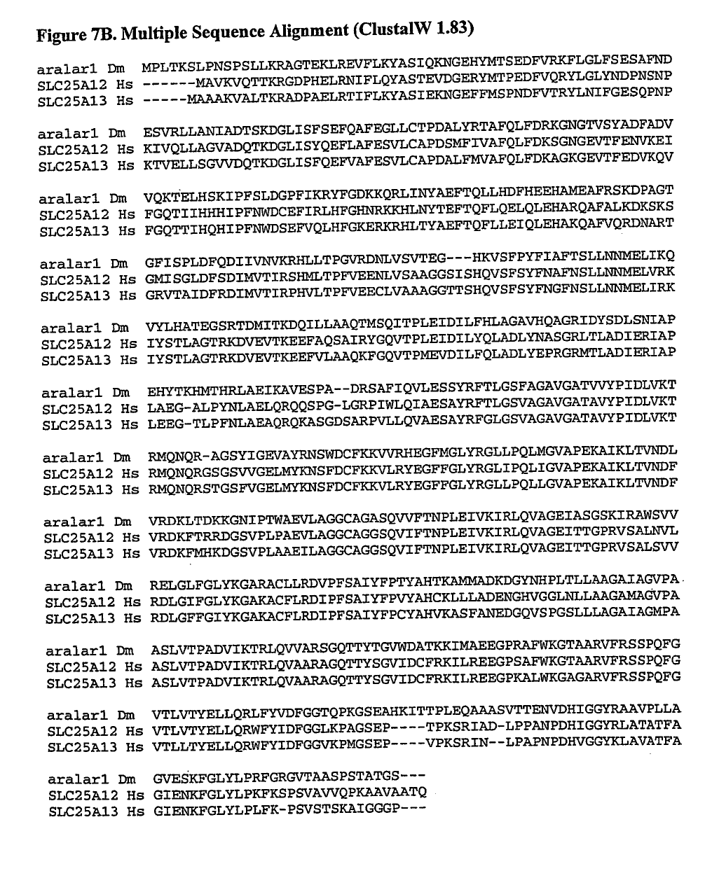

FIG. 7 shows the homology of Drosophila aralar 1 to human solute carrier family 25, members 11 and 12.

FIG. 7A shows the BLASTP search results for the aralar 1 gene product (Query) with the two best human homologous matches (Sbjct).

FIG. 7B shows the comparison of human and Drosophila proteins. ‘aralar1 Dm’ refers to Drosophila protein encoded by aralar 1, ‘SLC25A12 Hs’ refers to human solute carrier family 25, member 12, and ‘SLC25A13 Hs’ refers to human solute carrier family 25, member 13.

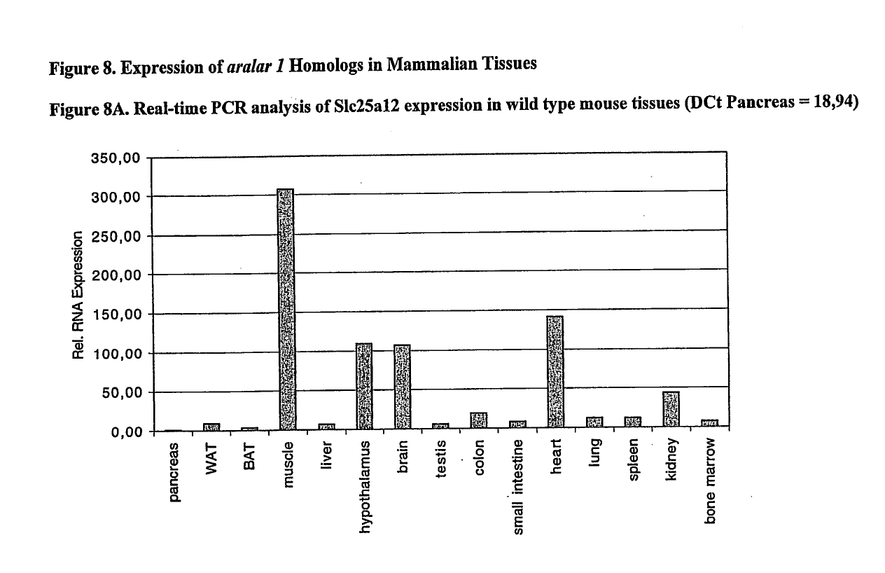

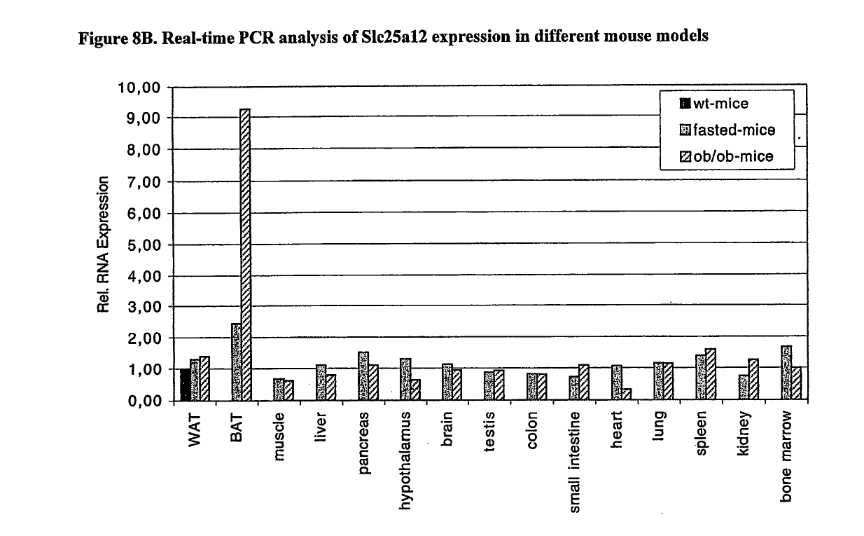

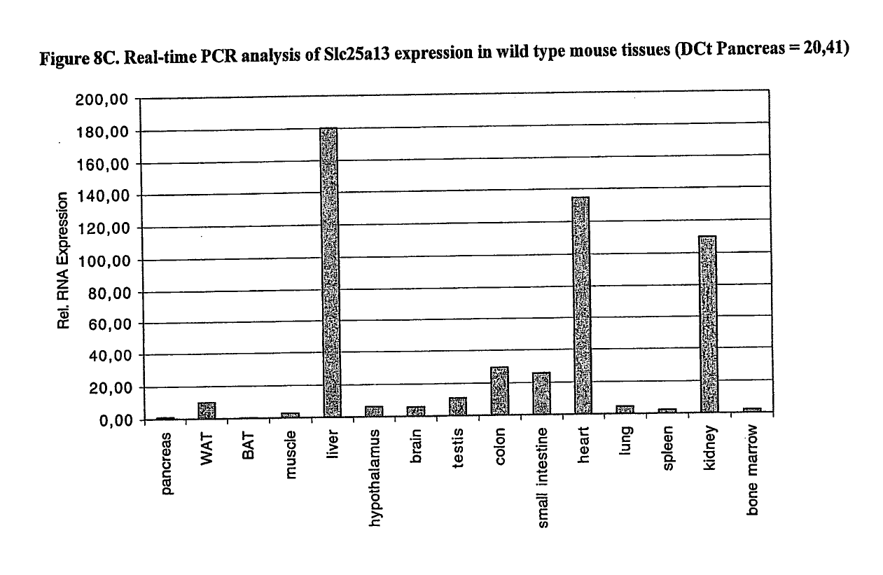

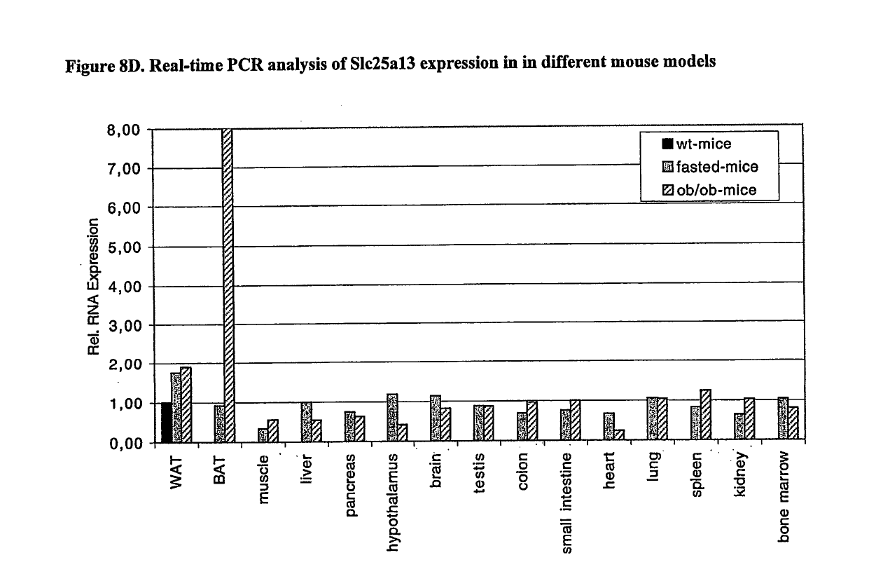

FIG. 8 shows the expression of the aralar 1 homologs in mammalian tissues.

FIG. 8A shows the real-time PCR analysis of solute carrier family 25, member 12 (Slc25a12) expression in wild-type mouse tissues.

FIG. 8B shows the real-time PCR analysis of Slc25a12 expression in different mouse models.

FIG. 8C shows the real-time PCR analysis of solute carrier family 25, member 13 (Slc25a13) expression in wild-type mouse tissues.

FIG. 8D shows the real-time PCR analysis of Slc25a13 expression in different mouse models.

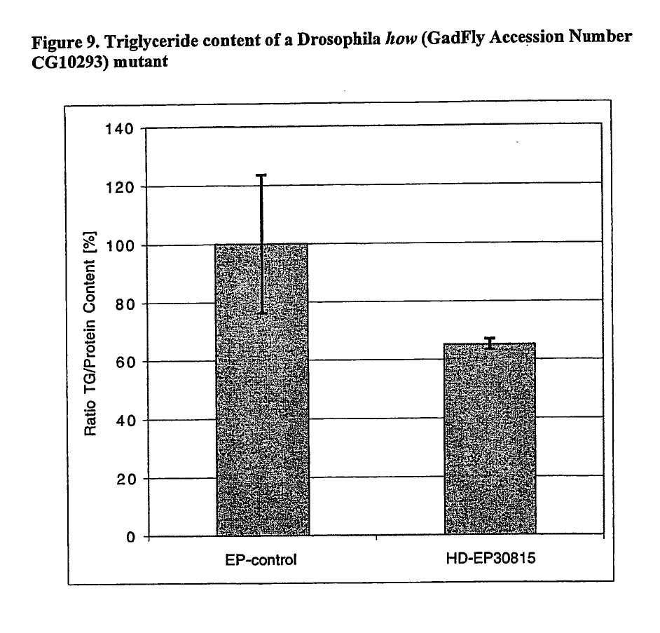

FIG. 9 shows the triglyceride content of a Drosophila how (Gadfly Accession Number CG10293) mutant. Shown is the change of triglyceride content of HD-EP(3)30815 flies caused by integration of the P-vector into the promoter of the how gene (referred to as ‘HD-EP30815’, column 2) in comparison to controls containing all flies of the EP collection (referred to as ‘EP-control’, column 1).

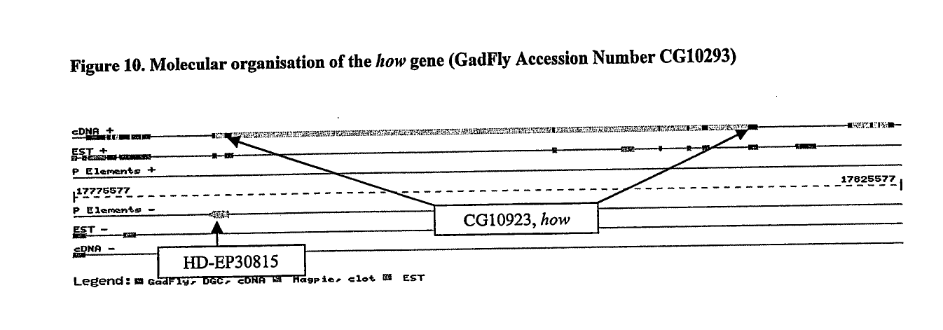

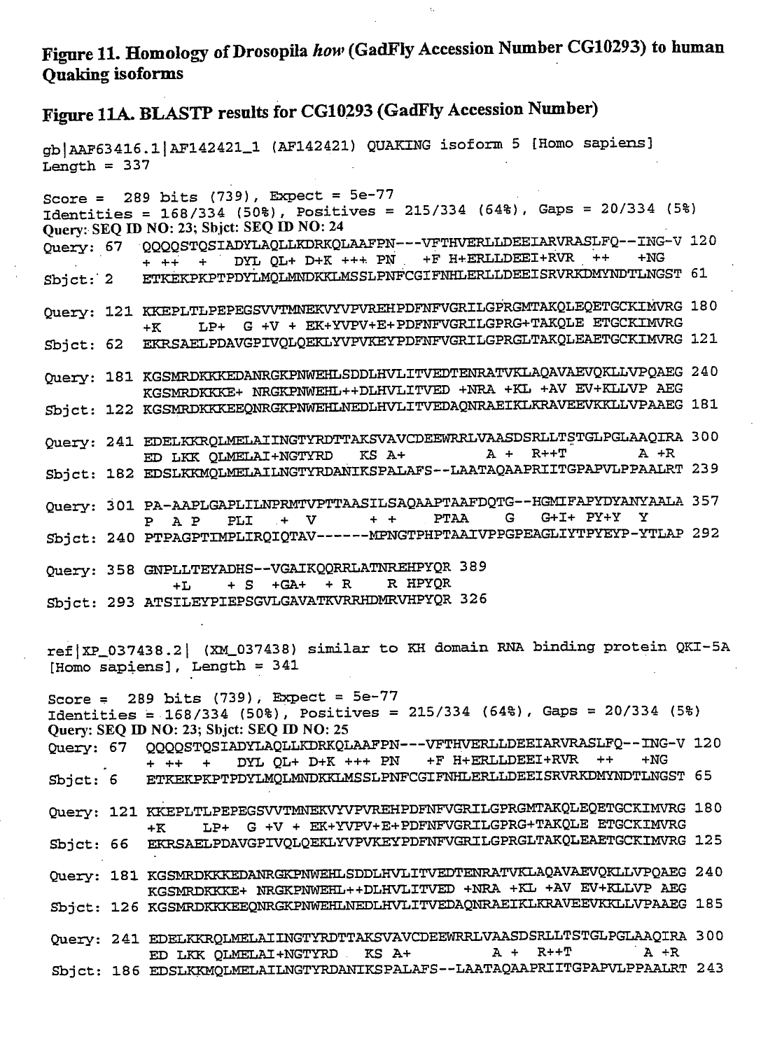

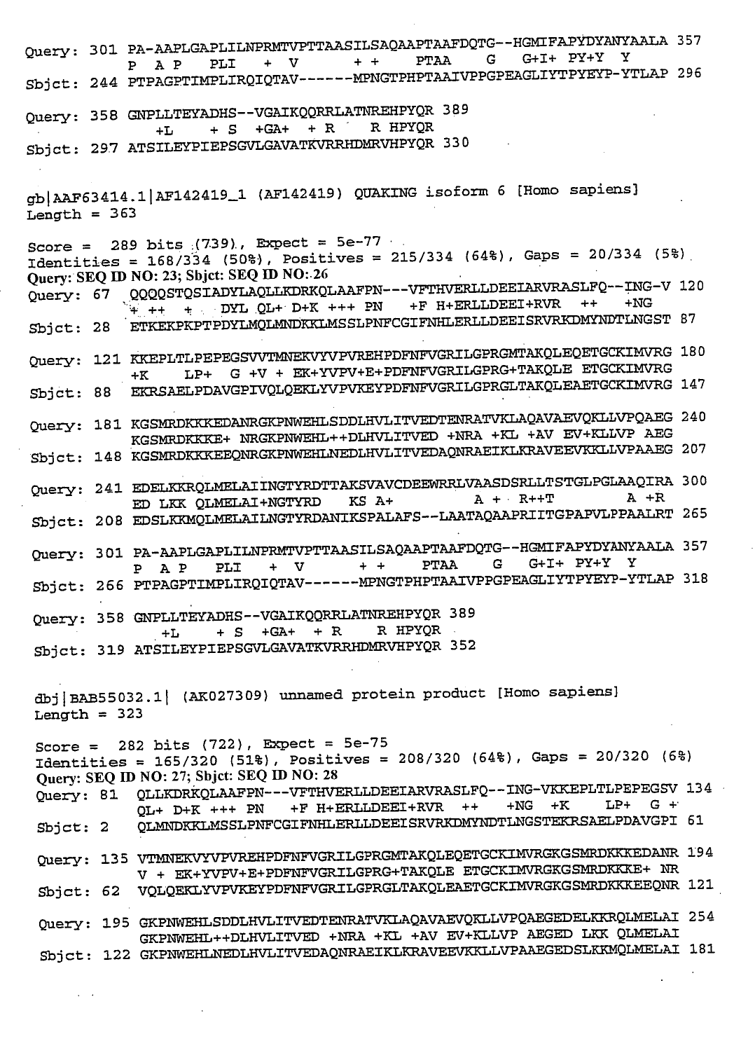

FIG. 10 shows the molecular organization of the mutated how (Gadfly Accession Number CG10293) gene locus.

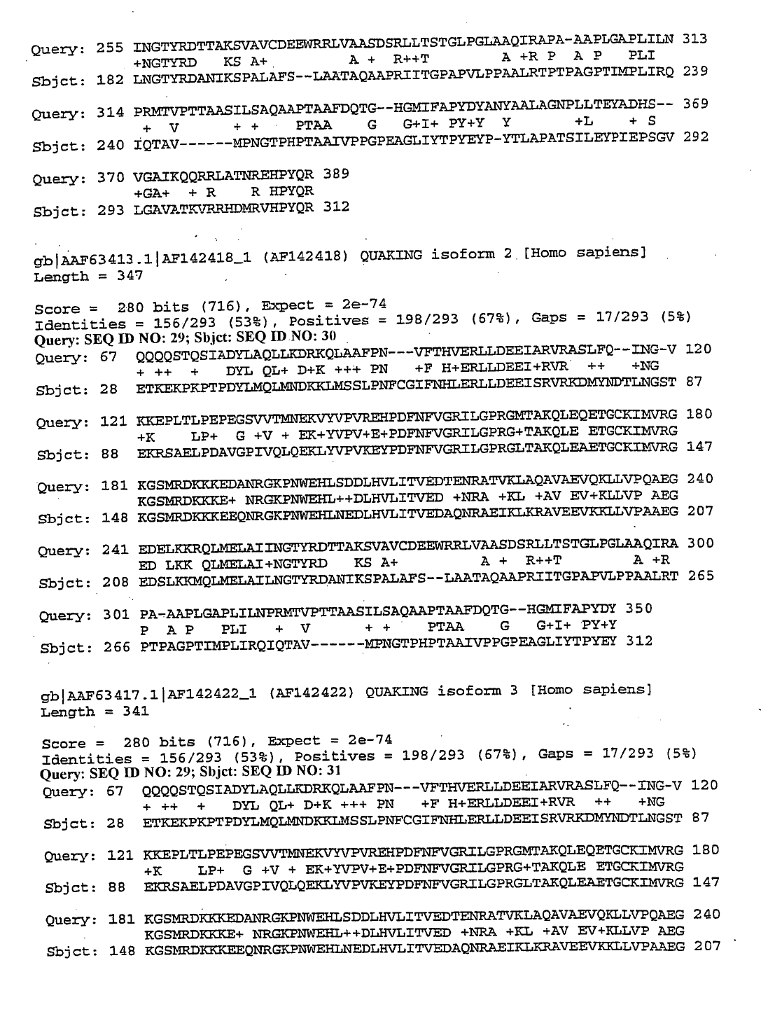

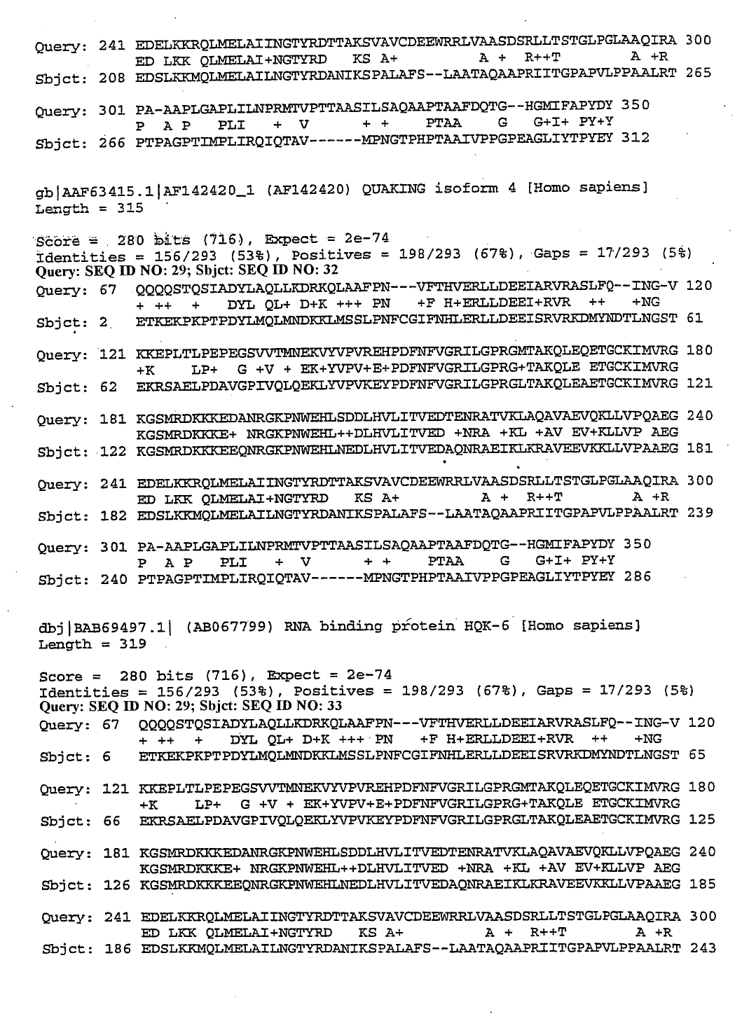

FIG. 11 shows the homology of Drosophila how (GadFly Accession Number CG10293) to the human quaking isoforms.

FIG. 11A shows the BLASTP search result for the how gene product (Query) with the twelve best human homologous matches (Sbjct).

FIG. 11B shows the comparison of human and Drosophila proteins. ‘CG10293 Dm’ refers to Drosophila protein encoded by CG10293, ‘QKI-6 Hs’ refers to human QUAKING isoform 6, ‘QKI-2 Hs’ refers to human QUAKING isoform 2, ‘QKI-3 Hs’ refers to human QUAKING isoform 3, and ‘HQK-7B Hs’ refers to human RNA binding protein HQK-7B.

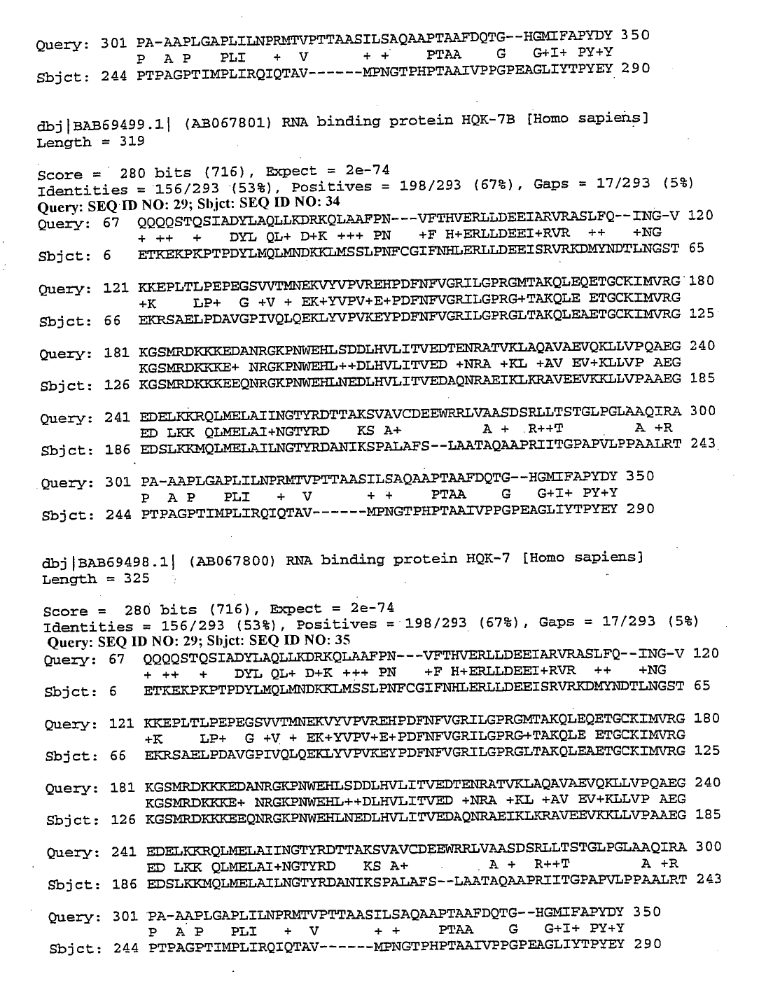

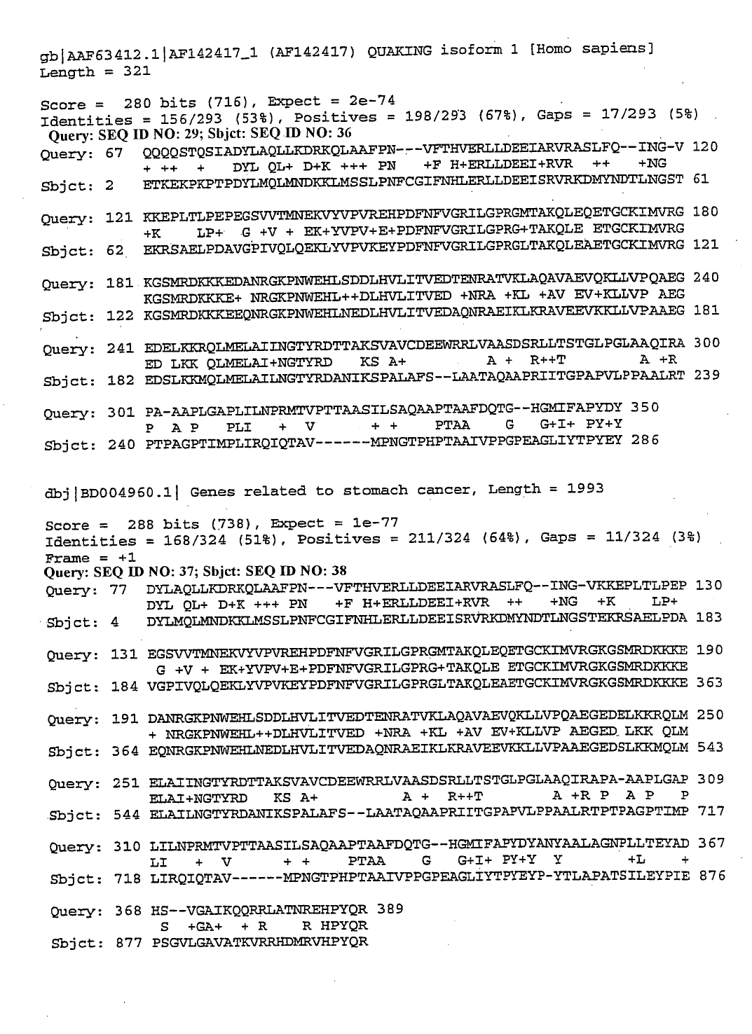

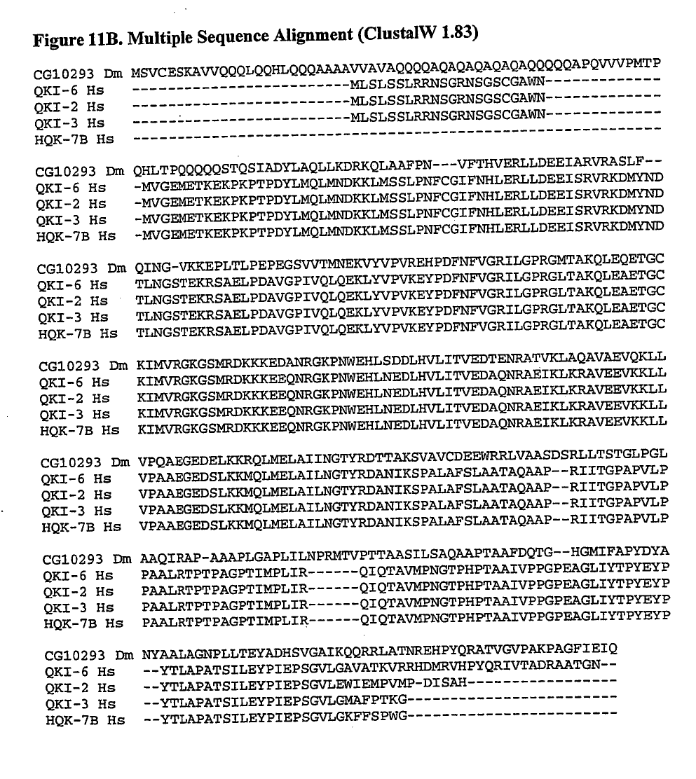

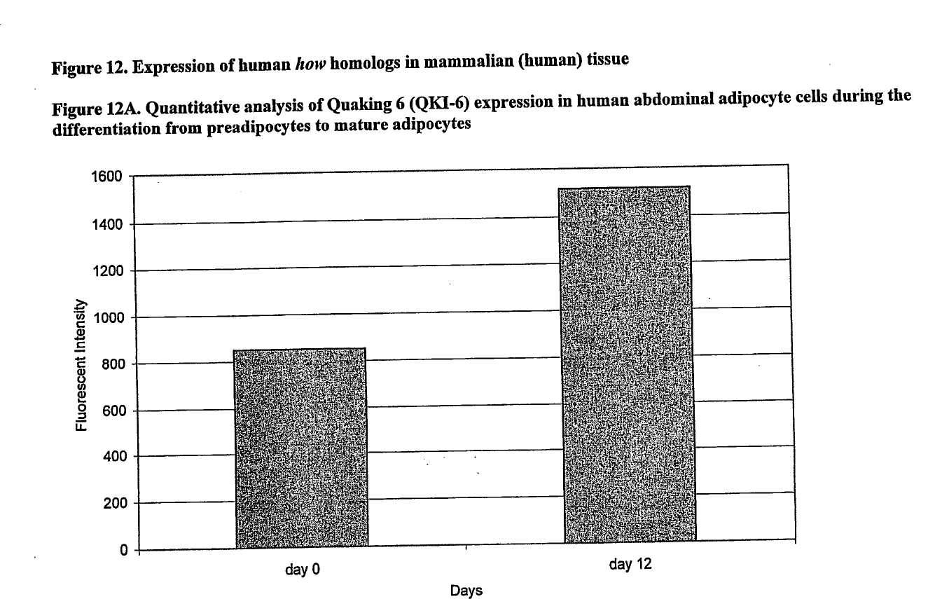

FIG. 12 shows the expression of how homologs in mammalian (human) tissue.

FIG. 12A shows the quantitative analysis of QUAKING 6 expression in human abdominal adipocyte cells, during the differentiation from preadipocytes to mature adipocytes.

FIG. 12B shows the quantitative analysis of RNA binding protein HQK-7B expression in human abdominal adipocyte cells, during the differentiation from preadipocytes to mature adipocytes.

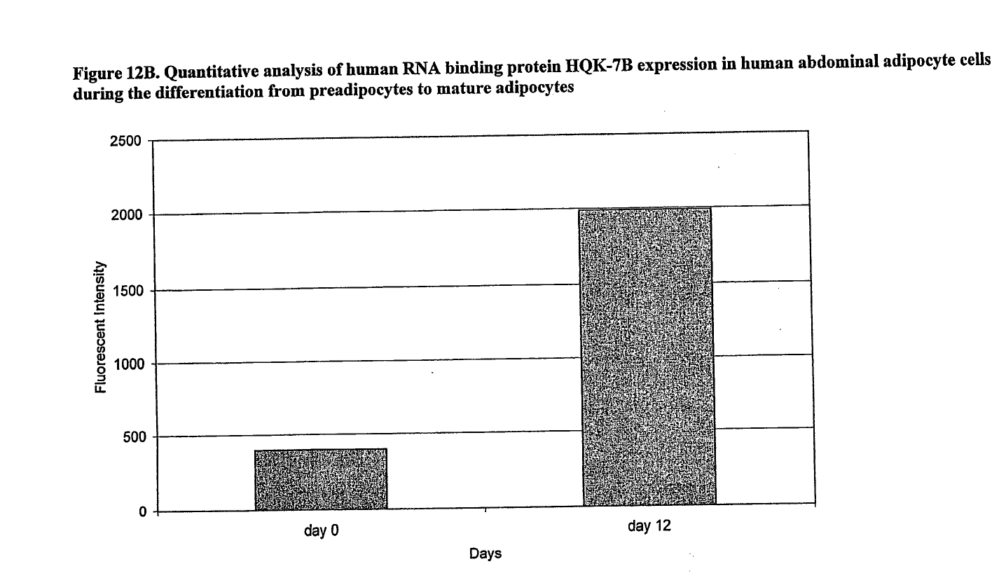

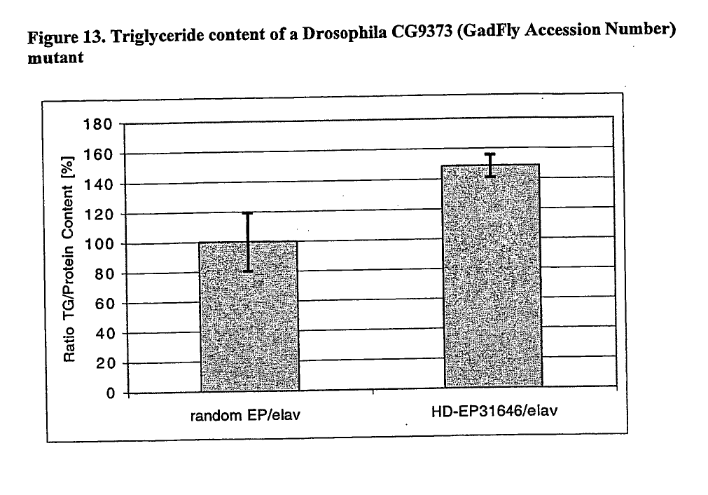

FIG. 13 shows the triglyceride content of a Drosophila Gadfly Accession Number CG9373 mutant. Shown is the change of triglyceride content of HD-EP(3)31646 flies caused by ectopic expression of the CG9373 gene mainly in the neurons of these flies (referred to as ‘HD-EP3646/elav’, column 2) in comparison to controls with integration of this vector (referred to as ‘random EP/elav’, column 1).

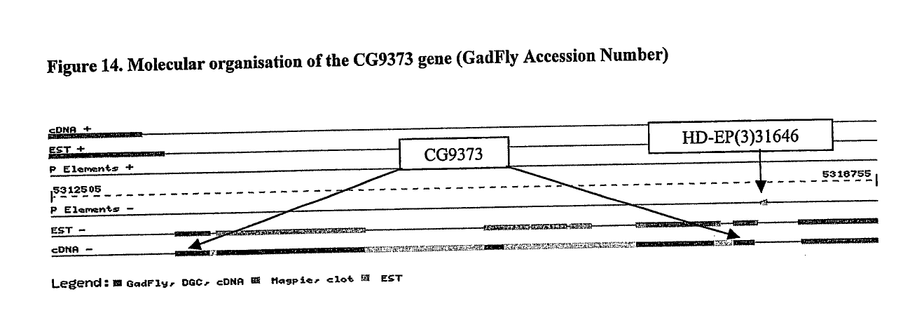

FIG. 14 shows the molecular organization of the mutated CG9373 (Gadfly Accession Number) gene locus.

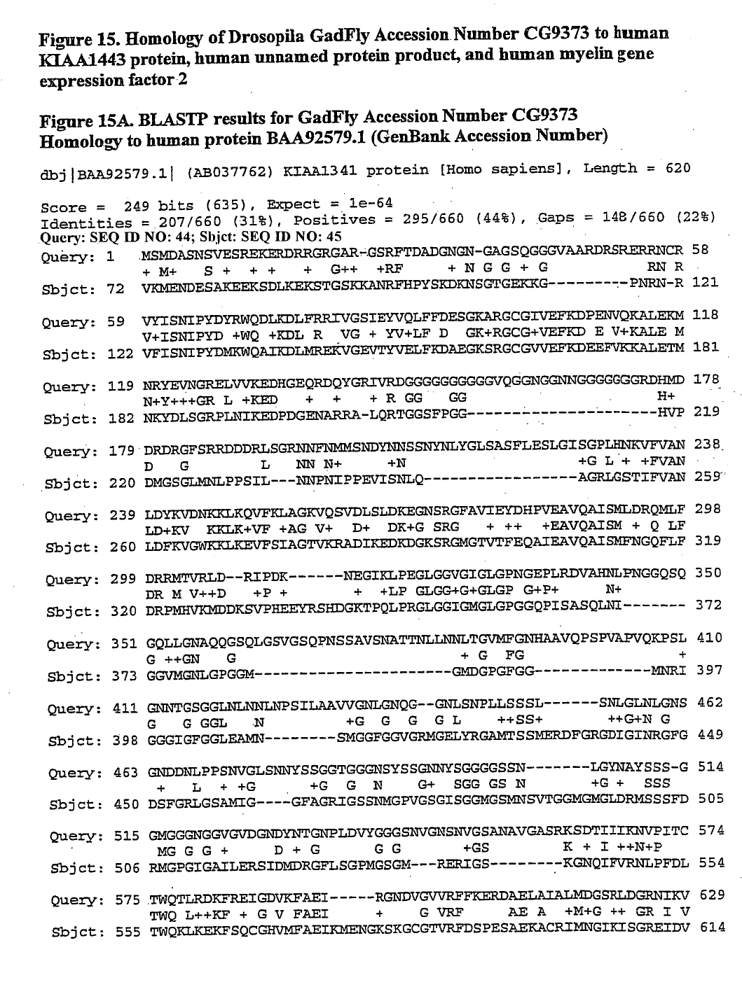

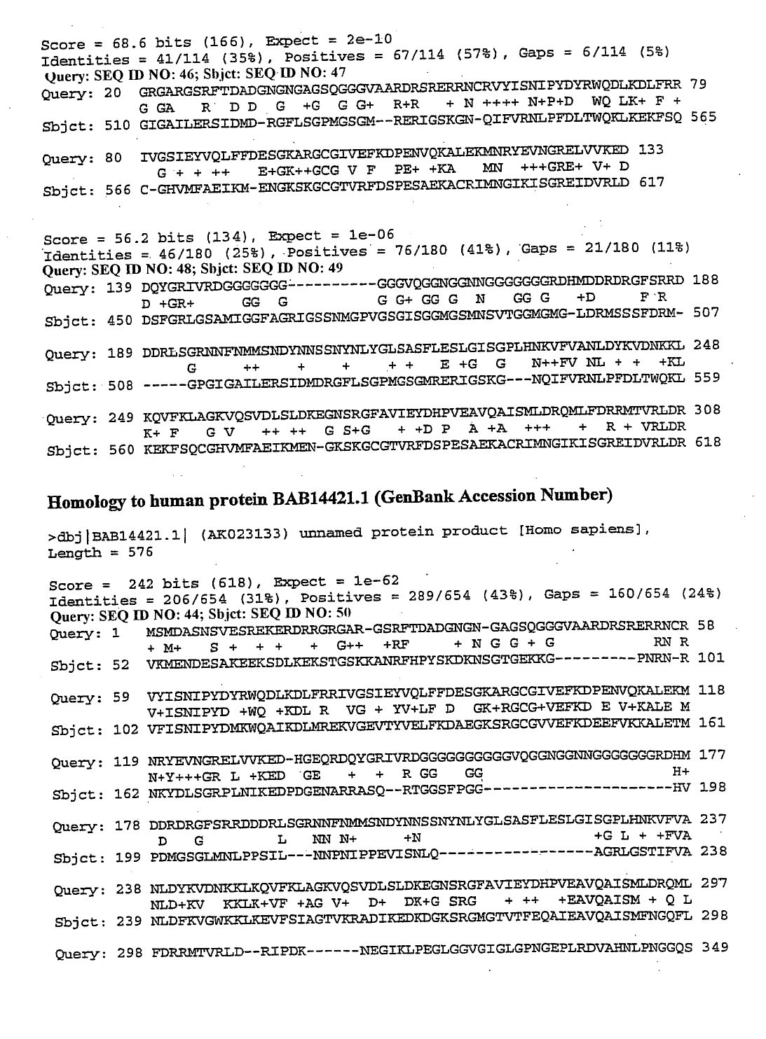

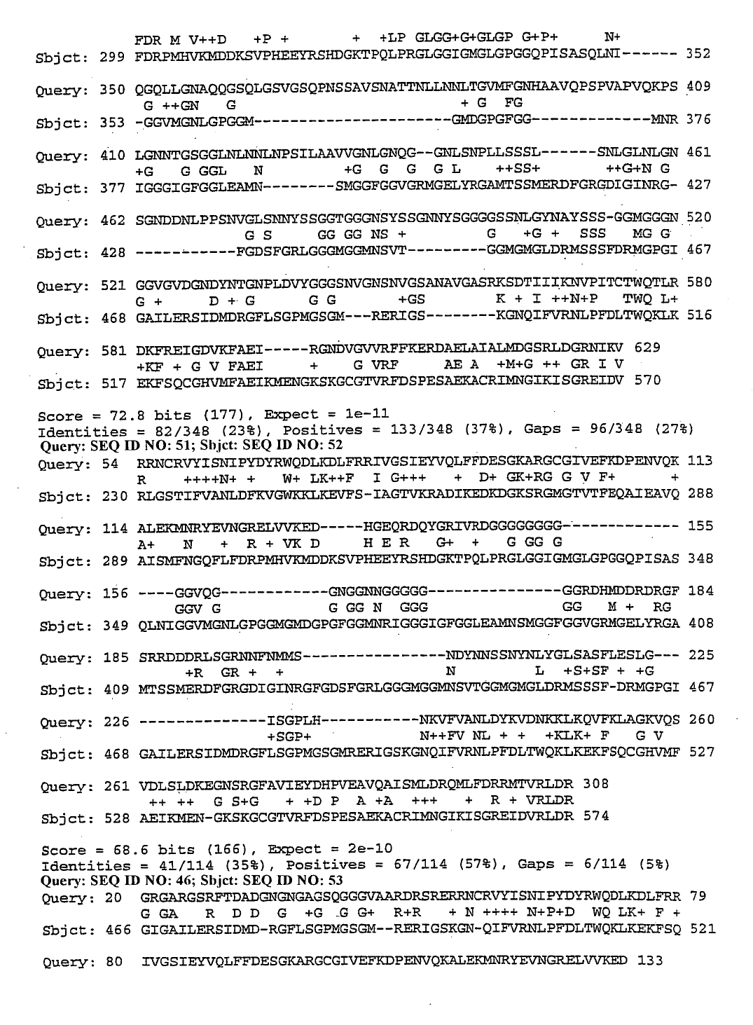

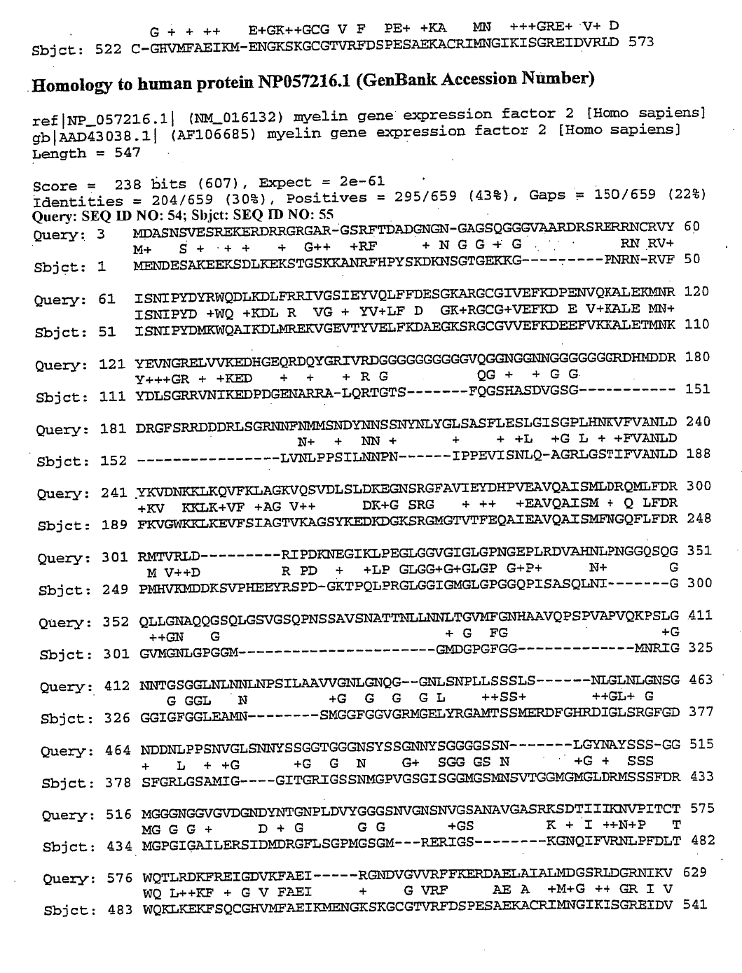

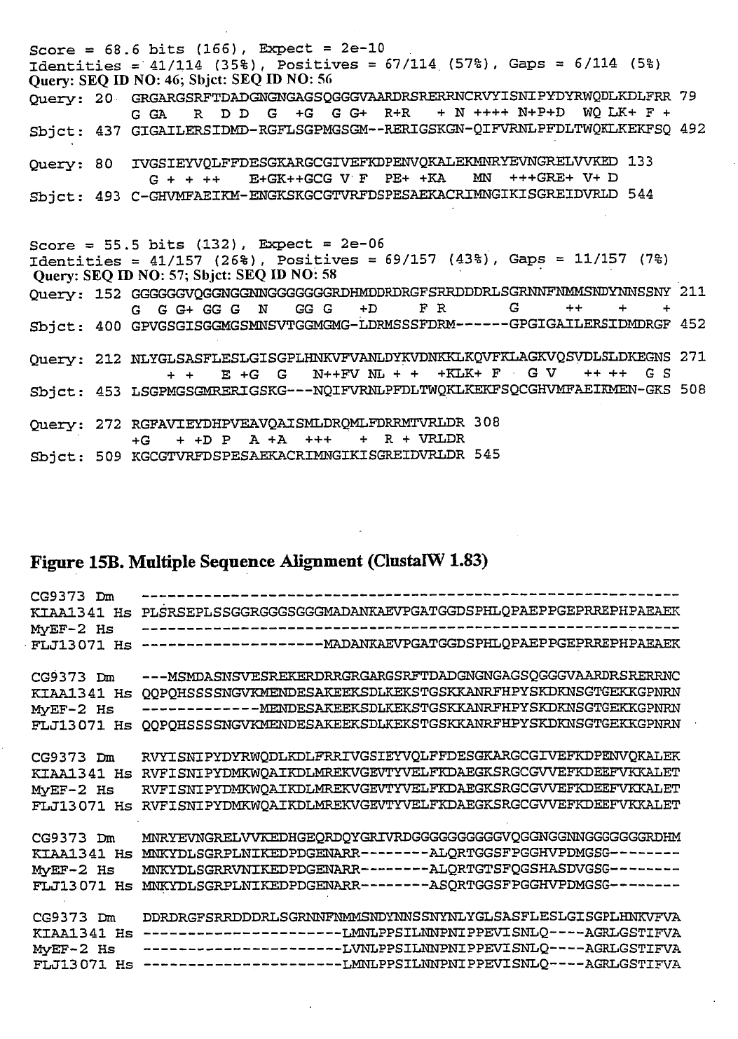

FIG. 15 shows the homology of Drosophila GadFly Accession Number CG9373 to human KIAA1443 protein, unnamed protein product, and myelin gene expression factor 2.

FIG. 15A shows the BLASTP search result for the CG9373 gene product (Query) with the three best human homologous matches (Sbjct).

FIG. 15B shows the comparison of human and Drosophila proteins. ‘CG9373 Dm’ refers to Drosophila protein encoded by CG9373, ‘KIAA1341 Hs’ refers to human KIAA1341 protein, ‘MyEF-2 Hs’ refers to human myelin gene expression factor 2, and ‘FLJ13071 Hs’ refers to human unnamed protein product FLJ13071.

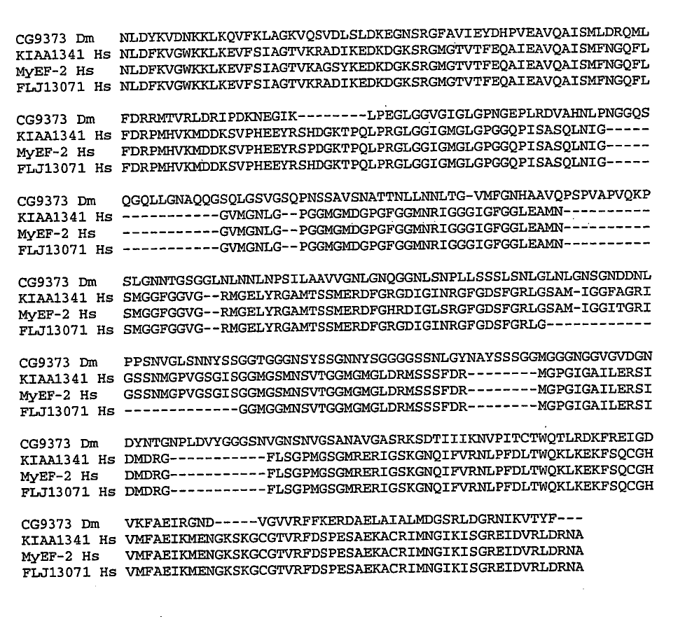

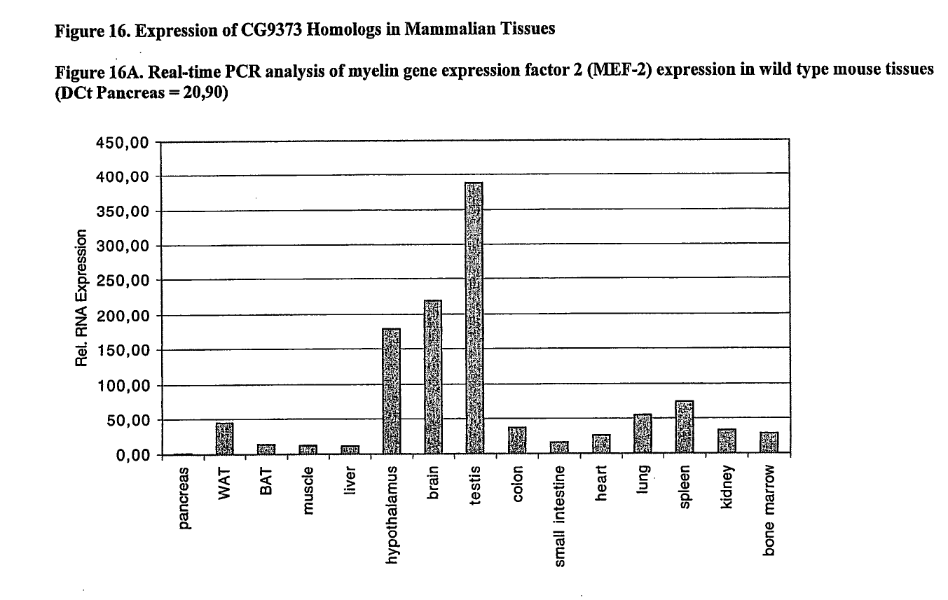

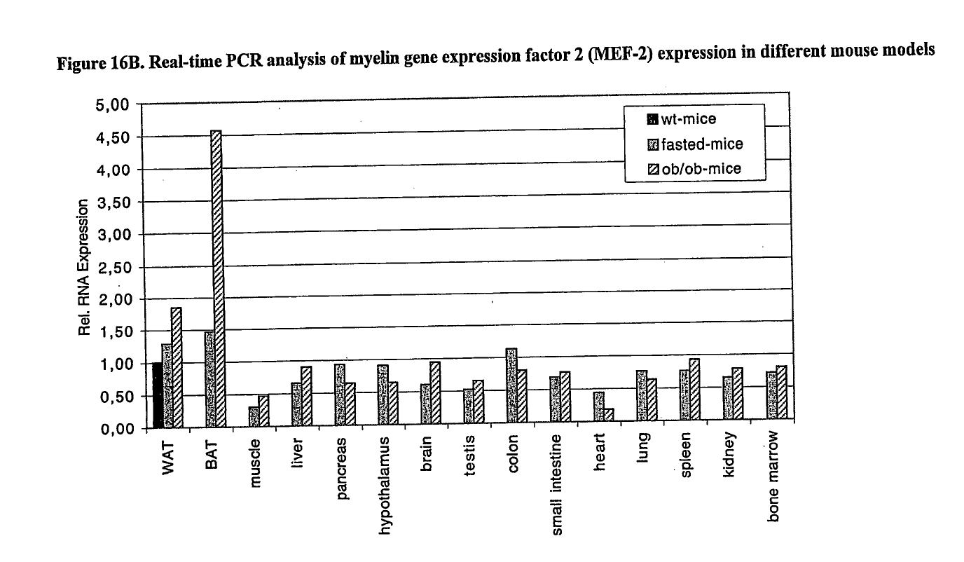

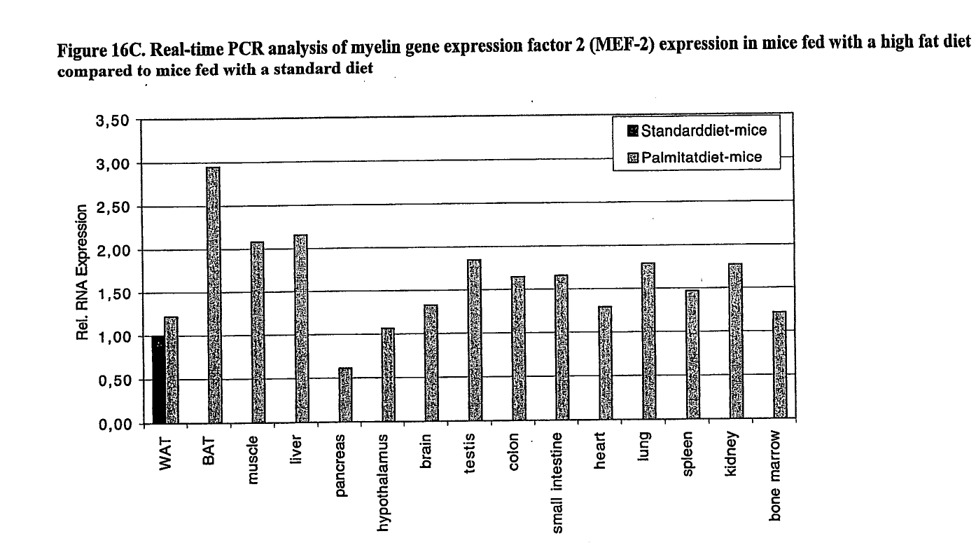

FIG. 16 shows the expression of the CG9373 homolog in mammalian tissues.

FIG. 16A shows the real-time PCR analysis of myelin gene expression factor 2 (MEF-2) expression in wild-type mouse tissues.

FIG. 16B shows the real-time PCR analysis of MEF-2 expression in different mouse models.

FIG. 16C shows the real-time PCR analysis of MEF-2 expression in mice fed with a high fat diet compared to mice fed with a standard diet.

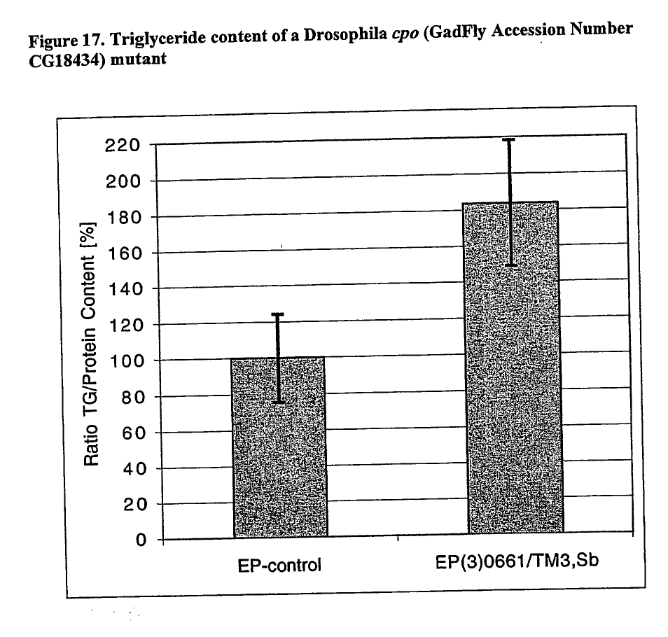

FIG. 17 shows the triglyceride content of a Drosophila cpo (Gadfly Accession Number CG18434) mutant. Shown is the change of triglyceride content of EP(3)0661 flies caused by integration of the P-vector into the promoter of the CG18434 gene (referred to as ‘EP(3)0661/Tm3,Sb’ column 2) in comparison to controls containing all flies of the EP collection (referred to as ‘EP-control’, column 1).

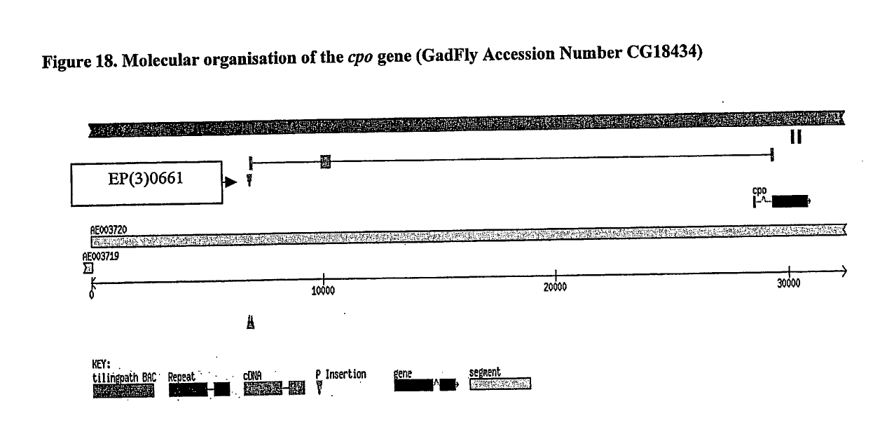

FIG. 18 shows the molecular organization of the mutated cpo (Gadfly Accession Number CG18434) gene locus.

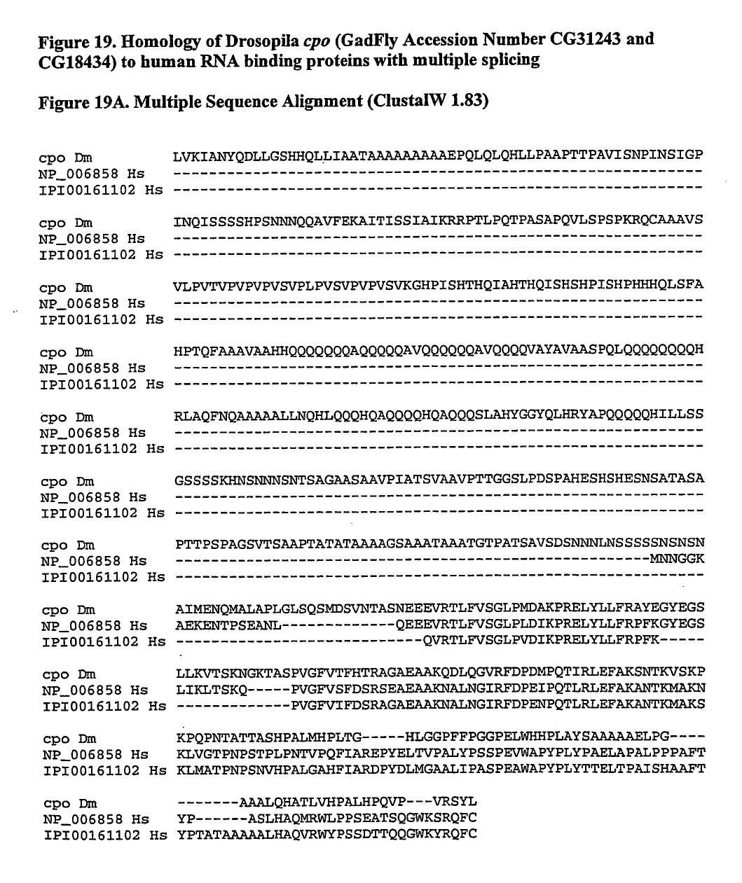

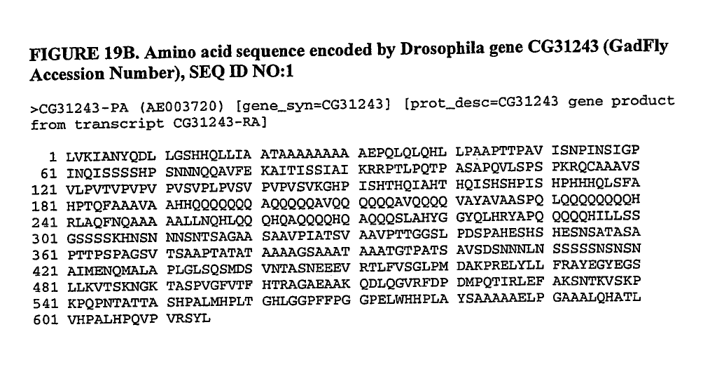

FIG. 19 shows the homology of Drosophila cpo to human RNA binding proteins with multiple splicing.

FIG. 19A shows the comparison of human and Drosophila proteins. ‘cpo Dm’ refers to Drosophila protein encoded by cpo, ‘NP_{—}006858 Hs’ refers to human RNA binding protein with multiple splicing (RBPMS), and ‘IPI001611’ refers to human RNA binding with multiple splicing (RBPMS) family member.

FIG. 19B shows the amino acid sequence encoded by Drosophila cpo gene (GadFly Accession Number CG31243, SEQ ID NO:1).

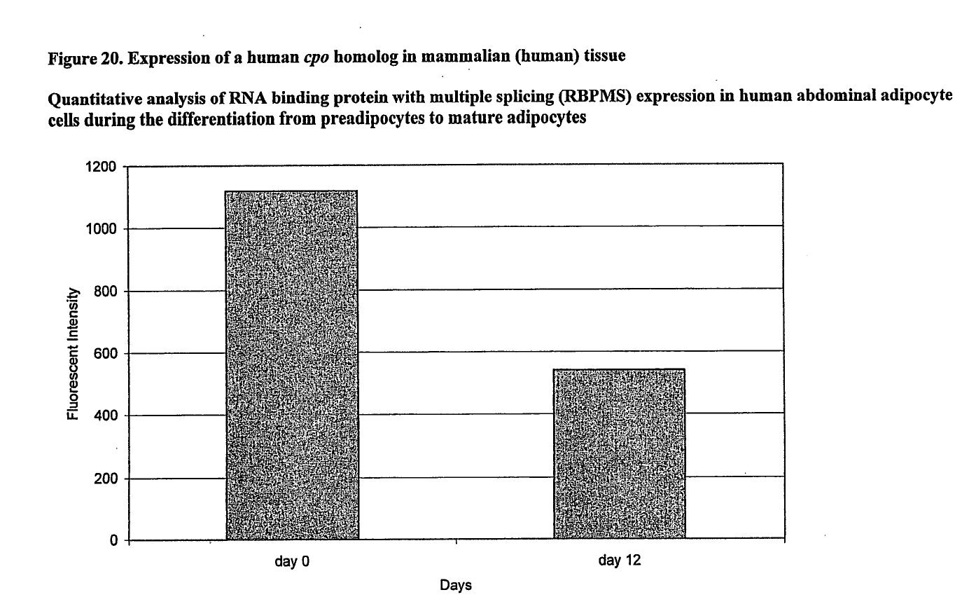

FIG. 20 shows the quantitative analysis of RNA binding protein with multiple splicing (RBPMS) expression in human abdominal adipocyte cells, during the differentiation from preadipocytes to mature adipocytes.

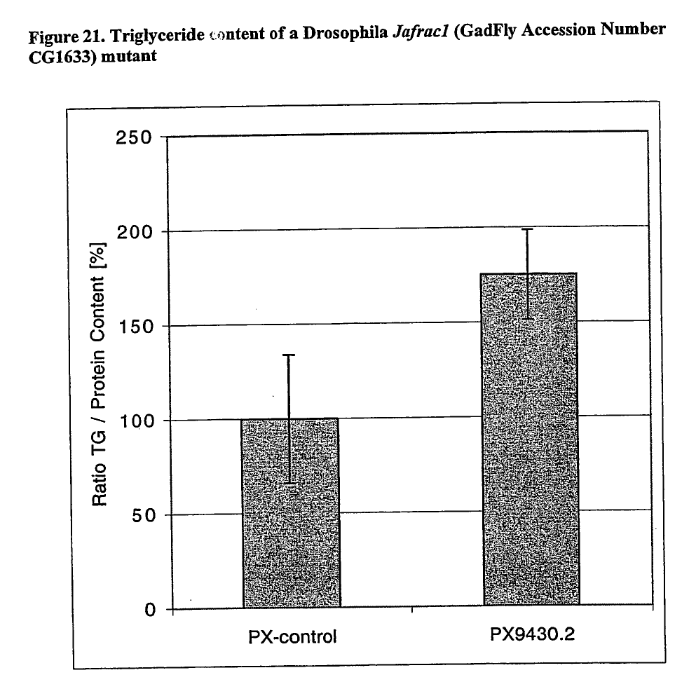

FIG. 21 shows the triglyceride content of a Drosophila Jafrac1 (Gadfly Accession Number CG1 633) mutant. Shown is the change of triglyceride content of PX9430.2 flies caused by integration of the P-vector into the leader of the Jafrac1 gene (referred to as ‘PX 9430.2’, column 2) in comparison to controls without integration of this vector, (herein referred to as ‘PX-control’, column 1).

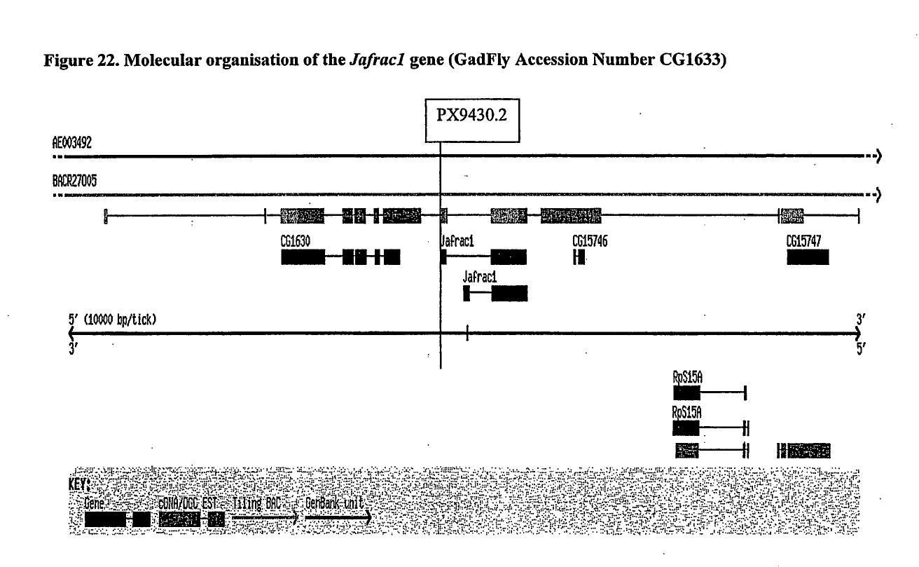

FIG. 22 shows the molecular organization of the mutated Jafrac1 (Gadfly Accession Number CG1633) gene locus.

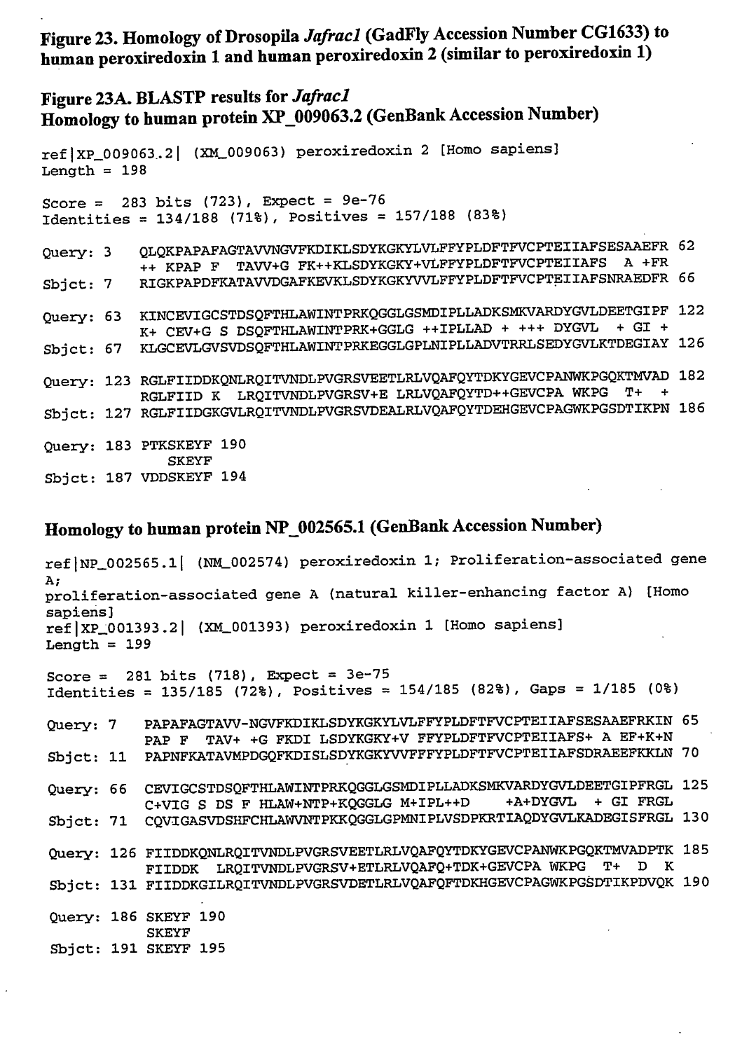

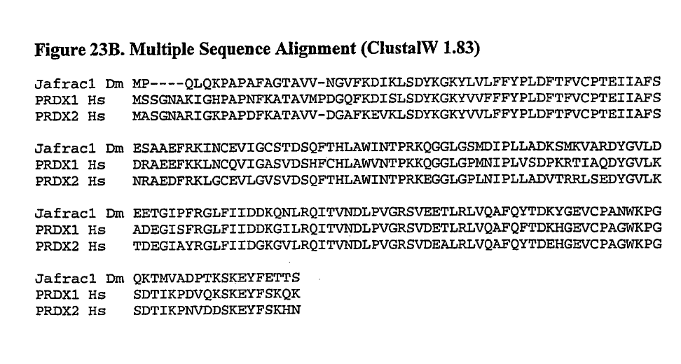

FIG. 23 shows the homology of Drosophila Jafrac1 (GadFly Accession Number CG1633) to human peroxiredoxin 1 and 2.

FIG. 23A shows the BLASTP search result for the Jafrac1 gene product (Query) with the best two human homologous matches (Sbjct).

FIG. 23B shows the comparison of human and Drosophila proteins. ‘Jafrac1 Dm’ refers to Drosophila protein encoded by Jafrac1, ‘PRDX1 Hs’ refers to human peroxiredoxin 1, and ‘PRDX2 Hs’ refers to human peroxiredoxin 2.

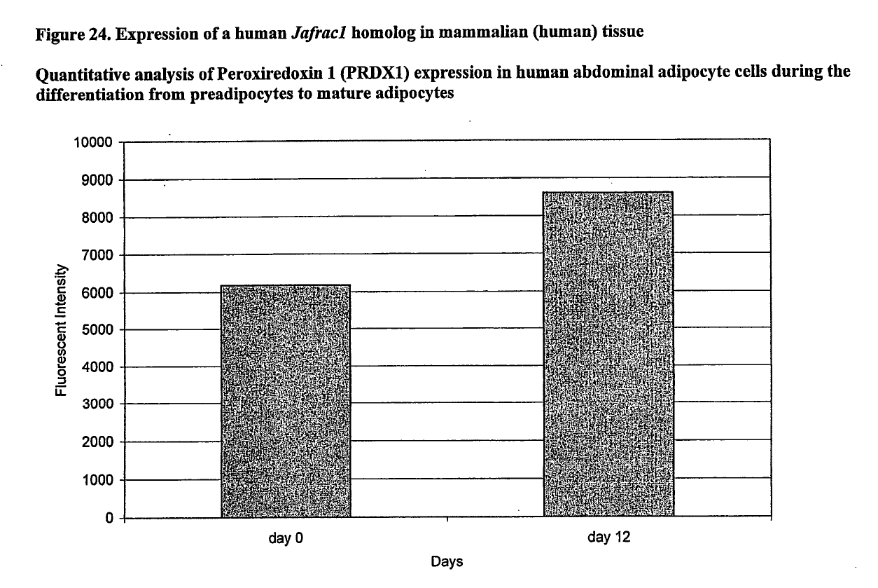

FIG. 24 shows the quantitative analysis of peroxiredoxin 1 (PRDX1) expression in human abdominal adipocyte cells, during the differentiation from preadipocytes to mature adipocytes.

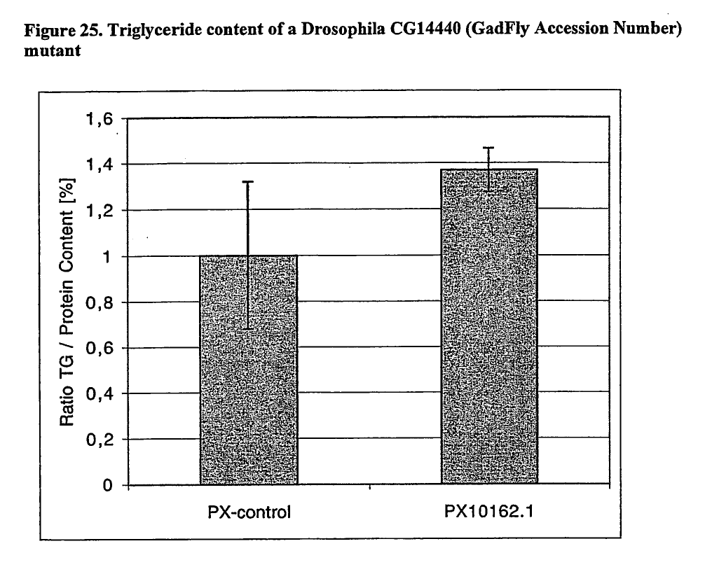

FIG. 25 shows the triglyceride content of a Drosophila Gadfly Accession Number CG14440 mutant. Shown is the change of triglyceride content of PX10162.1 flies caused by integration of the P-vector upstream of the CG14440 gene (referred to as ‘PX10162.1’, column 2) in comparison to controls without integration of this vector, (herein referred to as ‘PX-control’, column 1).

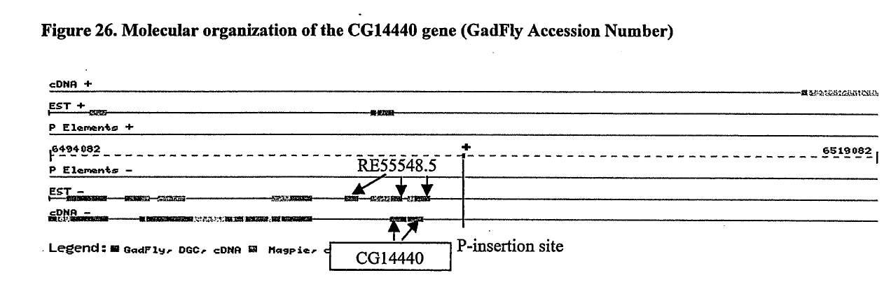

FIG. 26 shows the molecular organization of the mutated CG14440 (Gadfly Accession Number) gene locus.

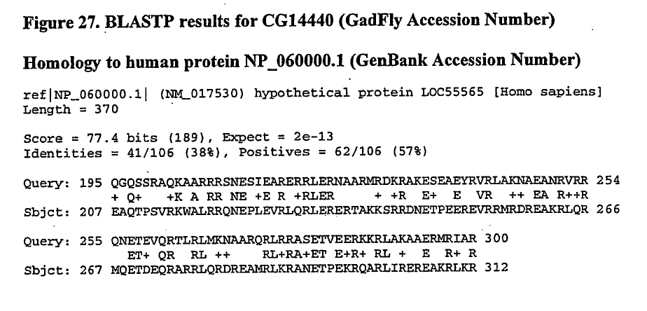

FIG. 27 shows the BLASTP search result for the CG14440 gene product (Query) with the best human homologous match (Sbjct).

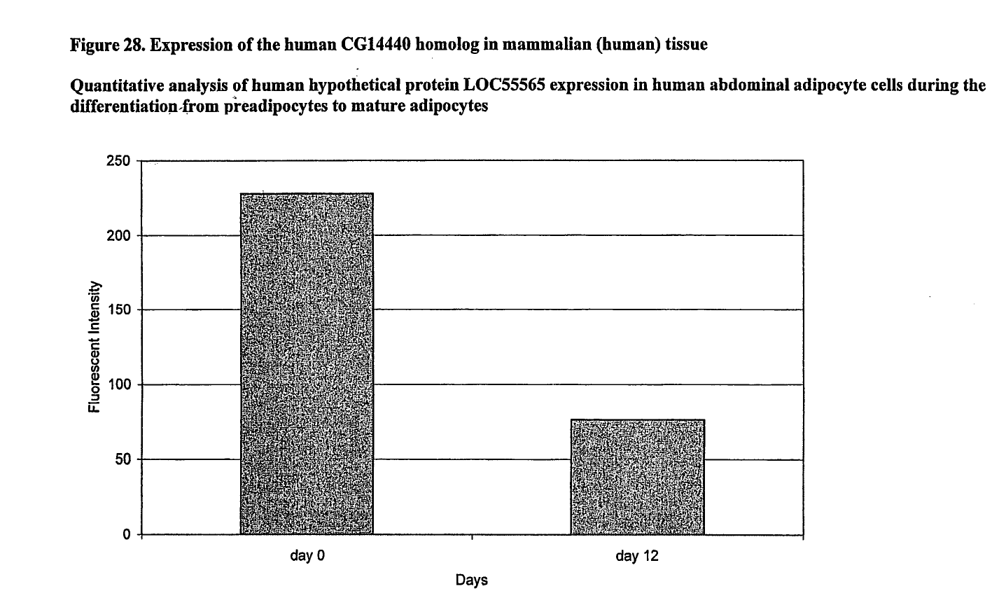

FIG. 28 shows the quantitative analysis of hypothetical protein LOC55565 expression in human abdominal adipocyte cells, during the differentiation from preadipocytes to mature adipocytes.

The examples illustrate the invention:

### EXAMPLE 1

### Measurement of Triglyceride Content in Drosophila

Mutant flies are obtained from proprietary and publicly available fly mutation stock collections. The flies are grown under standard conditions known to those skilled in the art. In the course of the experiment, additional feedings with bakers yeast (Saccharomyces cerevisiae) are provided. The average change of triglyceride content of Drosophila containing the EP-vectors in homozygous or heterozygous viable integration was investigated in comparison to control flies (see FIGS. 1, 5, 9, 13, and 17, 21, and 25). For determination of triglyceride, flies were incubated for 5 min at 90° C. (in case of PX9430.2 and PX10162.1 at 70° C.) in an aqueous buffer using a waterbath, followed by hot extraction. After another 5 min incubation at 90° C. (in case of PX9430.2 and PX10162.1 at 70° C.) and mild centrifugation, the triglyceride content of the flies extract was determined using Sigma Triglyceride (INT 336-10 or -20) assay by measuring changes in the optical density according to the manufacturer's protocol. As a reference protein content of the same extract was measured using BIO-RAD DC Protein Assay according to the manufacturer's protocol for the EP-lines. The assays were repeated several times.

The average triglyceride level of all flies of the EP collections (referred to as ‘EP-control’) is shown as 100% in the first columns in FIGS. 1, 5, 9, and 17, respectively. The average triglyceride level of about 50 lines of the PX collection (referred to as ‘PX-control’) is shown as 100% in the first column in FIGS. 21 and 25 (relative amount of triglyceride per fly). The average triglyceride level of all flies containing the elav-Gal4 vector (referred to as ‘random EP/elav’) is shown as 100% in the first column in FIG. 13. Standard deviations of the measurements are shown as thin bars.

HD-EP(3)31805 homozygous flies (column 2 in FIG. 1), EP(3)0661 heterozygous flies (column 2 in FIG. 17, referred to as ‘EP(3)0661/TM3,Sb’), PX9430.2 homozygous flies (column 2 in FIG. 21), and PX10162.1 homozygous flies (column 2 in FIG. 25) show constantly a higher triglyceride content than the controls. EP(3)3675 homozygous flies (column 2 in FIG. 5) and HD-EP(3)30815 homozygous flies (column 2 in FIG. 9) show constantly a lower triglyceride content than the controls. Therefore, the loss of gene activity in the loci where the EP-vectors or PX-vectors are viably integrated, is responsible for changes in the metabolism of the energy storage triglycerides.

HD-EP(3)31646 males were crossed to elav-Gal4 virgins. The offspring carries a copy of the HD-EP(3)31646 vector and a copy of the elav-Gal4 vector, leading to ectopic expression of adjacent genomic DNA sequences 3prime of the HD-EP(3)31646 integration locus, mainly in the neurons of these flies. The flies were analyzed in an assay measuring the triglyceride content of these flies. The result of the triglyceride content analysis is shown in FIG. 13. HD-EP(3)31646/elav flies show constantly a higher triglyceride content (column 2 in FIG. 13) than the control EP-collection that is crossed to elav-Gal4 (referred to as ‘random EP/elav’, column 1 in FIG. 13). Therefore, the gain of gene activity in the locus, where the EP-vector of HD-EP(3)31646 flies is integrated in the promoter of the CG9373 gene, is responsible for changes in the metabolism of the energy storage triglycerides.

### EXAMPLE 2

### Identification of Drosophila Genes Associated with Regulation of Metabolism

Nucleic acids encoding the proteins of the present invention were identified using a plasmid-rescue technique. Genomic DNA sequences were isolated that are localized adjacent to the EP vector (herein HD-EP(3)31805, EP(3)3675, HD-EP(3)30815, HD-EP(3)31646, EP(3)0661, PX9430.2, or PX10162.1) integration. Using those isolated genomic sequences public databases like Berkeley Drosophila Genome Project (GadFly) were screened thereby identifying the integration sites of the vectors, and the corresponding genes. The molecular organization of these gene loci is shown in FIGS. 2, 6, 10, 14, 18, 22, and 26.

In FIGS. 2, 10, 14, and 26, genomic DNA sequence is represented by the assembly as a dotted black line in the middle that includes the integration sites of the vectors for lines HD-EP(3)31805, HD-EP(3)30815, HD-EP(3)31646, or PX10162.1. Numbers represent the coordinates of the genomic DNA. The upper parts of the figures represent the sense strand “+”, the lower parts represent the antisense strand “−”. The insertion sites of the P-elements in the Drosophila lines are shown as triangles or boxes in the “P-elements +”, “P-elements −”, or middle lines. Transcribed DNA sequences (ESTs) are shown as grey bars in the “EST +” and/or the “EST −” lines, and predicted cDNAs are shown as bars in the “cDNA +” and/or “cDNA −” lines. Predicted exons of the cDNAs are shown as dark grey bars and introns are shown as light grey bars.

In FIGS. 6, 18, and 22, genomic DNA sequence is represented by the assembly as a thin black scaled double-headed arrow in the middle that includes the integration sites of the vectors for lines EP(3)3675, EP(3)0661, or PX9430.2. Numbers and ticks represent the length of the genomic DNA (1000 base pairs per tick in FIG. 6, 10000 base pairs per tick in FIGS. 18 and 22). The upper part of the figure represents the sense strand, the lower part represent the antisense strand. The grey arrows in the upper part of FIGS. 6 and 22, and the dark grey box in the topmost part of FIG. 18 represent BAC clones, the black arrows in the topmost part of FIGS. 6 and 22, and the light grey box in the middle of FIG. 18 represent the sections of the chromosomes or GenBank units. The insertion sites of the P-elements in the Drosophila lines are shown as grey triangles in FIGS. 6 and 18, and as black vertical line in FIG. 22. The P-insertion sites are labeled. Grey bars, linked by black lines represent cDNA sequences. Predicted genes are shown as black bars (exons), linked by black lines (FIGS. 6 and 22) or light grey serrated lines (FIG. 18) (introns), and are labeled (see also key at the bottom of the figures).

The HD-EP(3)31805 vector is homozygous viable integrated 3 base pairs 5′ of a Drosophila gene in antisense orientation, identified as GadFly Accession Number CG7956. The chromosomal localization site of the integration of the vector of HD-EP(3)31805 is at gene locus 3R, 93E4. In FIG. 2, the coordinates of the genomic DNA are starting at position 17260000 on chromosome 3R, ending at position 17270000. The insertion site of the P-element in Drosophila HD-EP(3)31805 line is shown in the “P Elements −” line and is labeled. The predicted cDNA of the CG7956 gene is shown in the “cDNA +” line and is labeled.

The EP(3)3675 vector is homozygous viable integrated into an intron of a Drosophila gene in sense orientation, identified as aralar1 (GadFly Accession Number CG2139). The chromosomal localization site of the integration of the vector of EP(3)3675 is at gene locus 3R, 99F6. In FIG. 6, the insertion site of the P-element in Drosophila EP(3)3675 line is shown in the as triangle in the lower part of the figure and labeled with an arrow. The predicted transcription variants of the Drosophila aralar1 gene (GadFly Accession Number CG2139) are shown as black boxes, linked with thin black lines.

The HD-EP(3)30815 vector is homozygous viable integrated into the promoter of a Drosophila gene in antisense orientation, identified as how (GadFly Accession Number CG10293). The chromosomal localization site of the integration of the vector of HD-EP(3)30815 is at gene locus 3R, 94A1-2. In FIG. 10, the coordinates of the genomic DNA are starting at position 17775577 on chromosome 3R, ending at position 17775577. The insertion site of the P-element in Drosophila HD-EP(3)30815 line is shown in the “P-elements −” line. The predicted cDNA of the how gene is shown in the “cDNA +” line and is labeled.

The HD-EP(3)31646 vector is homozygous viable integrated into the promoter region of a Drosophila gene in sense orientation, identified as GadFly Accession Number CG9373. The chromosomal localization site of the integration of the vector of HD-EP(3)31646 is at gene locus 3R, 85D25. In FIG. 14, the coordinates of the genomic DNA are starting at position 5312505 on chromosome 3R, ending at position 5318755. The insertion site of the P-element in Drosophila HD-EP(3)31646 line is shown in the “P-elements −” line. The predicted cDNA of the CG9373 gene is shown in the “cDNA −” line and is labeled.

The EP(3)0661) vector is homozygous lethal/heterozygous viable integrated into the promoter of RE30936.5 in sense orientation, representing an EST-clone of a Drosophila gene, identified as cpo (GadFly Accession Numbers CG18434 and CG31243). The chromosomal localization site of the integration of the vector of EP(3)0661 is at gene locus 3R, 90D1. In FIG. 18, the insertion site of the P-element in Drosophila EP(3)0661 line is shown as triangle in the upper part of the figure and labeled with an arrow. The predicted cDNA of the cpo gene is shown in the upper part of the figure and is labeled.

The PX9430.2 vector is homozygous viable integrated into the leader sequence of a Drosophila gene, identified as Jafrac1 (GadFly Accession Number CG1 633). The chromosomal localization site of the integration of the vector of PX9430.2 is at gene locus X, 11E6. In FIG. 22, the insertion site of the P-element in Drosophila PX9430.2 line is shown as vertical labeled line. The predicted transcript variants of the Drosophila Jafrac1 gene are shown in the upper part of the figure and are labeled.

The PX10162.1 vector is homozygous viable integrated upstream of the 5′-end of a Drosophila gene, identified as GadFly Accession Number CG14440. The chromosomal localization site of the integration of the vector of PX10162.1 is at gene locus X, 6C7. In FIG. 26, the coordinates of the genomic DNA are starting at position 6494082 on chromosome X, ending at position 6519082. The insertion site of the P-element in Drosophila PX10162.1 line is shown as “+” on the dotted middle line. The predicted cDNA of CG14440 shown in the “cDNA −” line and is labeled, the corresponding EST is shown in the “EST −” line and is labeled.

Expression of the genes described above could be affected by integration of the vectors into the transcription units, leading to a change in the amount of the energy storage triglycerides.

### EXAMPLE 3

### Identification of Human Homologous Genes and Proteins

The Drosophila genes and proteins encoded thereby with functions in the regulation of triglyceride metabolism were further analysed using the BLAST algorithm searching in publicly available sequence databases and mammalian homologs were identified (see Table 1 and FIGS. 3, 7, 11, 15, 19, 23, and 27).

TABLE 1
Human homologs of the Drosophila (Dm) genes

| Dm gene | Homo sapiens homologous genes and proteins | | |
|---|---|---|---|
| Acc. No. | Accession Number | | |
| Name | cDNA | Protein | Name |
| CG7956 | NM_014937 | NP_055752 | Sac domain-containing inositol |
| | | | phosphatase 2 (SAC2); KIAA0966 |
| CG2139 | NM_003705 | NP_003696 | solute carrier family 25 (mitochondrial |
| aralar1 | | | carrier, Aralar), member 12 (SLC25A12) |
| | NM_014251 | NP_055066 | solute carrier family 25, member 13 |
| | | | (citrin) (SLC25A13) |
| CG10293 | AF142419 | AAF63414 | QUAKING isoform 6 (QUAKING) |
| how | AF142418 | AAF63413 | QUAKING isoform 2 (QUAKING) |
| | AF142422 | AAF63417 | QUAKING isoform 3 (QUAKING) |
| | AB067801 | BAB69499 | RNA binding protein HQK-7B |
| CG9373 | AB037762 | BAA92579 | KIAA1341 protein |
| | AK023133 | BAB14421 | unnamed protein product FLJ13071 |
| | NM_016132 | NP_057216 | myelin gene expression factor 2 (MEF-2) |
| CG31243 | NM_006867 | NP_006858 | RNA binding protein with multiple |
| CG18434 | | | splicing (RBPMS) |
| cpo | ENSG00000 | ENSP00000 | RNA binding with multiple splicing |
| | 166831 | 300069 | (RBPMS) family member |
| CG1633 | NM_002574 | NP_002565 | peroxiredoxin 1 (PRDX1) |
| Jafrac1 | BC000452 | AAH00452 | protein similar to thioredoxin peroxidase |
| | | | 1 |
| CG14440 | NM_017530 | NP_060000 | hypothetical protein LOC55565 |
| | | | (LOC55565) |

CG7956, aralar1, how, CG9373, cpo, Jafrac1, or CG14440 homologous proteins and nucleic acid molecules coding therefore are obtainable from insect or vertebrate species, e.g. mammals or birds. Particularly preferred are nucleic acids as described in Table 1.

The present invention is describing polypeptides comprising the amino acid sequences of the proteins of the invention. Comparisons (Clustal W 1.83 analysis, see for example Thompson J. D. et al., (1994) Nucleic Acids Res. 22(22):4673-4680; Thompson J. D., (1997) Nucleic Acids Res 25(24):4876-4882; Higgins, D. G. et al., (1996) Methods Enzymol. 266:383-402) between the respective proteins of different species (human and Drosophila) were conducted. Gaps in the alignment are represented as −. Based upon homology, the Drosophila proteins of the invention and each homologous protein or peptide may share at least some activity.

As shown in FIG. 3, gene product of Drosophila GadFly Accession Number CG7956 is 52% homologous to human Sac domain-containing inositol phosphatase (SAC2, also referred to as KIAA0966 protein; GenBank Accession Number NP_{—}055752.1 for the protein, NM_{—}014937 for the cDNA). CG7956 also shows homology to mouse protein ENSMUSP00000045910 (ENSEMBL Accession Number).

Human solute carrier family 25 (mitochondrial carrier, Aralar), member 12 is also referred to as GenBank Accession Number XP_{—}010876.3 for the protein, XM_{—}010876 for the cDNA. As shown in FIG. 7A, the gene product of Drosophila aralar 1 is 74% homologous to human solute carrier family 25 (mitochondrial carrier, Aralar), member 12 and 73% homologous to human solute carrier family 25, member 13 (citrin). aralar 1 also shows homology to mouse solute carrier family 25 (mitochondrial carrier; adenine nucleotide translocator), member 13 (GenBank Accession Number NP_{—}056644.1).

As shown in FIG. 11A, gene product of Drosophila how is 64% homologous to human QUAKING isoform 5 (GenBank Accession Number AAF63416.1 for the protein, AF142421 for the cDNA), 64% homologous to human protein similar to KH domain RNA binding protein QKI-5A (GenBank Accession Number XP_{—}037438.2 for the protein, XM_{—}037438 for the cDNA), 64% homologous to QUAKING isoform 6 (GenBank Accession Number AAF63414.1 for the protein, AF142419 for the cDNA), 64% homologous to unnamed protein product (GenBank Accession Number BAB55032.1 for the protein, AK027309 for the cDNA), 67% homologous to QUAKING isoform 2 (GenBank Accession Number AAF63413.1 for the protein, AF142418 for the cDNA), 67% homologous to QUAKING isoform 3 (GenBank Accession Number AAF63417.1 for the protein, AF142422 for the cDNA), 67% homologous to QUAKING isoform 4 (GenBank Accession Number AAF6341 5.1 for the protein, AF142420 for the cDNA), 67% homologous to QUAKING isoform 3 (GenBank Accession Number AAF63417.1 for the protein, AF142422 for the cDNA), 67% homologous to RNA binding protein HQK-6 (GenBank Accession Number BAB69497.1 for the protein, AB067799 for the cDNA), 67% homologous to RNA binding protein HQK-7B (GenBank Accession Number BAB69499.1 for the protein, AB067801 for the cDNA), 67% homologous to RNA binding protein HQK-7 (GenBank Accession Number BAB69498.1 for the protein, AB067800 for the cDNA), 67% homologous to QUAKING isoform 1 (GenBank Accession Number AAF63412.1 for the protein, AF142417 for the cDNA), and 64% to genes related to stomach cancer (GenBank Accession Number BD004960.1. Drosophila how also shows homology to mouse KH domain RNA binding protein QKI-7B (GenBank Accession Number AAC63042.1).

As shown in FIG. 15A, gene product of Drosophila GadFly Accession Number CG9373 is 44% homologous to human KIAA1341 protein (GenBank Accession Number BAA92579.1 for the protein, AB037762 for the cDNA), 43% homologous to human unnamed protein product (GenBank Accession Number BAB14421.1 for the protein, AK023133 for the cDNA), and 43% to myelin gene expression factor 2 (GenBank Accession Number NP_{—}057216.1 for the protein, NM_{—}016132 for the cDNA. CG9373 also shows homology to mouse myelin gene expression factor (GenBank Accession Number AAL90778.1).

Drosophila cpo is also referred to as SEQ ID NO:1 in FIG. 19B. Human RNA-binding protein gene with multiple splicing (RBPMS) is also referred to as GenBank Accession Number XP_{—}047075.1 for the protein, XM_{—}047075 for the cDNA, and human gene similar to RNA-binding protein with multiple splicing is also referred to as GenBank Accession Number XP_{—}091097 for the protein, XM_{—}091097 for the cDNA. As shown in FIG. 19A, the gene product of Drosophila CG31243 is 62% homologous to human RNA-binding protein with multiple splicing and 59% homologous to human protein similar to RNA-binding protein with multiple splicing at the C-terminal part, respectively.

As shown in FIG. 23A, gene product of Drosophila Jafrac1 is 83% homologous to human peroxiredoxin 2 (GenBank Accession Number XP_{—}009063.2 for the protein, XM_{—}009062 for the cDNA) and 82% homologous to human peroxiredoxin 1 (GenBank Accession Number NP_{—}002565.1 for the protein, NM_{—}002574 for the cDNA). CG1633 also shows homology to mouse thioredoxin dependent peroxide reductase 2 (GenBank Accession Number NP_{—}035164.1) and to mouse peroxiredoxin 4 (GenBank Accession Number NP_{—}048044.1).

As shown in FIG. 27, gene product of Drosophila GadFly Accession Number CG14440 is 57% homologous to human hypothetical protein LOC55565 (GenBank Accession Number NP_{—}060000.1 for the protein, NM_{—}017530 for the cDNA). CG14440 also shows homology to mouse protein similar to hypothetical protein LOC55565 (GenBank Accession Number AAH23180.1).

The human Jafrac1 homologous protein peroxiredoxin 1 is also referred to as natural killer cell enhancing factor A in U.S. Pat No. 5,610,286-A. The human Jafrac1 homologous protein peroxiredoxin 2 is also referred to as amino acid sequence of the acid form of peroxyredoxin TDX1 in Patent Number FR2798672-A1. The human CG14440 homologous protein is also referred to as human polypeptide SEQ ID NO 3381 in Patent Number WO200153312-A1.

### EXAMPLE 4

### Genetic Adipose Pathway Screen

Adipose (adp) is a protein that has been described as regulating, causing or contributing to obesity in an animal or human (see WO 01/96371). Transgenic flies containing a wild type copy of the adipose cDNA under the control of the Gal4/UAS system were generated (Brand and Perrimon, 1993, Development 118:401-415; for adipose cDNA, see WO 01/96371). Chromosomal recombination of these transgenic flies with an eyeless-Gal4 driver line has been used to generate a stable recombinant fly line over-expressing adipose in the developing Drosophila eye. Animals receiving transgenic adipose activity under these conditions developed into adult flies with a visible change of eye phenotype. Virgins of the recombinant driver line were crossed with males of the mutant EP-line collection in single crosses and kept for preferably 12 to 15 days at 29° C. The offspring was checked for modifications of the eye phenotype (enhancement or suppression). Mutations changing the eye phenotype affect genes that modify adipose activity. The inventors have found that the fly line HD-EP(3)35715 is a suppressor of the eye-adp-Gal4 induced eye phenotype. This result is strongly suggesting an interaction of the cpo gene with adipose since the integration of HD-EP(3)35715 was found to be located at the cpo locus. This is supporting the function of cpo and homologous proteins in the regulation of the energy homeostasis.

### EXAMPLE 5

### dUCPy Modifier Screen

Expression of Drosophila uncoupling protein dUCPy in a non-vital organ like the eye (Gal4 under control of the eye-specific promoter of the “eyeless” gene) results in flies with visibly damaged eyes. This easily visible eye phenotype is the basis of a genetic screen for gene products that can modify UCP activity.

Parts of the genomes of the strain with Gal4 expression in the eye and the strain carrying the pUAST-dUCPy construct were combined on one chromosome using genomic recombination. The resulting fly strain has eyes that are permanently damaged by dUCPy expression. Flies of this strain were crossed with flies of a large collection of mutagenized fly strains. In this mutant collection a special expression system (EP-element, Ref.: Rorth P, Proc Natl Acad Sci USA 1996, 93(22):12418-22) is integrated randomly in different genomic loci. The yeast transcription factor Gal4 can bind to the EP-element and activate the transcription of endogenous genes close the integration site of the EP-element. The activation of the genes therefore occurs in the same cells (eye) that overexpress dUCPy. Since the mutant collection contains several thousand strains with different integration sites of the EP-element it is possible to test a large number of genes whether their expression interacts with dUCPy activity. In case a gene acts as an enhancer of UCP activity the eye defect will be worsened; a suppressor will ameliorate the defect.

Using this screen a gene with suppressing activity was discovered that was found to be the cpo gene in Drosophila.

### EXAMPLE 6

### Expression of the Polypeptides in Mammalian (Mouse) Tissues

For analyzing the expression of the polypeptides disclosed in this invention in mammalian tissues, several mouse strains (preferrably mice strains C57BI/6J, C57BI/6 ob/ob and C57BI/KS db/db which are standard model systems in obesity and diabetes research) were purchased from Harlan Winkelmann (33178 Borchen, Germany) and maintained under constant temperature (preferrably 22° C.), 40 per cent humidity and a light/dark cycle of preferrably 14/10 hours. The mice were fed a standard chow (for example, from ssniff Spezialitäten GmbH, order number ssniff M-Z V1126-000). For the fasting experiment (“fasted wild type mice”), wild type mice were starved for 48 h without food, but only water supplied ad libitum (see, for example, Schnetzler et al., (1993) J Clin Invest 92(1):272-280, Mizuno et al., (1996) Proc Natl Acad Sci USA 93(8):3434-3438). Animals were sacrificed at an age of 6 to 8 weeks. The animal tissues were isolated according to standard procedures known to those skilled in the art, snap frozen in liquid nitrogen and stored at −80° C. until needed.

RNA was isolated from mouse tissues using Trizol Reagent (for example, from Invitrogen, Karlsruhe, Germany) and further purified with the RNeasy Kit (for example, from Qiagen, Germany) in combination with an DNase-treatment according to the instructions of the manufacturers and as known to those skilled in the art. Total RNA was reverse transcribed (preferrably using Superscript II RNaseH− Reverse Transcriptase, from Invitrogen, Karlsruhe, Germany) and subjected to Taqman analysis preferrably using the Taqman 2× PCR Master Mix (from Applied Biosystems, Weiterstadt, Germany; the Mix contains according to the Manufacturer for example AmpliTaq Gold DNA Polymerase, AmpErase UNG, dNTPs with dUTP, passive reference Rox and optimized buffer components) on a GeneAmp 5700 Sequence Detection System (from Applied Biosystems, Weiterstadt, Germany).

Taqman analysis was performed preferrably using the following primer/probe pairs:
For the amplification of Sac domain-containing inositol phosphatase 2 (sac2) (SEQ ID NO: 1): 5′-CCT GGA TCG CAC CAA CG-3′; mouse sac2 reverse primer (SEQ ID NO: 2): 5′-TTA AGC TGC TGT TCC ATG ACC A-3′; Taqman probe (SEQ ID NO: 3): (5/6-FAM) TCC AGG CTG CCA TAG CGC GC (5/6-TAMRA)
For the amplification of mouse solute carrier family 25 (mitochondrial carrier, Aralar) member 12 (Slc25a12) (SEQ ID NO: 4): 5′-CCT GCC AAC CCT GAT CAC A-3′; mouse Slc25a12 reverse primer (SEQ ID NO: 5): 5′-TTT CAA TGC CAG CGA AAG TG-3′; Taqman probe (SEQ ID NO: 6): (5/6-FAM) CGG TGG CTA CAG ACT TGC CAC GG (5/6-TAMRA)
For the amplification of mouse solute carrier family 25 (mitochondrial carrier; adenine nucleotide translocator), member 13 (Slc25a13) (SEQ ID NO: 7): 5′-AGC GGT GGT TCT ATG TCG ATT T-3′; mouse Slc25a13 reverse primer (SEQ ID NO: 8): 5′-CGG GAT TTA GGA ACC GGC T-3′; Taqman probe (SEQ ID NO: 9): (5/6-FAM) AGG CGT GAA GCC CGT GGG ATC T (5/6-TAMRA)
For the amplification of mouse myelin gene expression factor 2 (mef2) (SEQ ID NO: 10): 5′-ACA AGG ATG GCA AGA GCA GAG-3′; mouse mef2 reverse primer (SEQ ID NO: 11): 5′-ATG GAA ATT GCT TGG ACT GCT T-3′; Taqman probe (SEQ ID NO: 12): (5/6-FAM) CAT GGG CAC TGT CAC TTT TGA GCA GG (5/6-TAMRA)

In the figures the relative RNA-expression is shown on the Y-axis. In FIGS. 4A and B, 8A, B, C, and D, and 16A, B, and C, the tissues tested are given on the X-axis. “WAT” refers to white adipose tissue, “BAT” refers to brown adipose tissue.

As shown in FIG. 4A, real time PCR (Taqman) analysis of the expression of the Sac domain-containing inositol phosphatase 2 (SAC2) RNA in mammalian (mouse) tissues revealed that SAC2 is highly expressed in hypothalamus, brain, WAT, spleen and kidney. FIG. 4B shows that SAC2 is upregulated in BAT and pancreas of fasted animals as well as ob/ob mice. The arcuate nucleus in the hypothalamus is the region in the brain that regulates feeding behaviour. The high expression level of SAC2 in the hypothalamus and WAT strongly suggests that this gene plays a central role in energy homeostasis. This is supported by the upregulation of SAC2 in BAT and the pancreas of two animal models used to study metabolic disorders.

As shown in FIG. 8A, real time PCR (Taqman) analysis of the expression of the solute carrier family 25, member 12 (Slc25a12) RNA in mammalian (mouse) tissues revealed that Slc25a12 is highly expressed in muscle, hypothalamus, brain and heart. As shown in FIG. 8B, Slc25a12 is nine-fold upregulated in BAT of ob/ob mice and more than two-fold upregulated in BAT of fasted animals. Slc25a12 is nearly three-fold downregulated in the heart of ob/ob mice. As shown in FIG. 8C, solute carrier family 25, member 13 (Slc25a13) is highy expressed in liver, heart and kidney of wild type animals. As shown in FIG. 8D, Slc25a13 is strongly upregulated in BAT of ob/ob mice and more than four-fold downregulated in heart tissue of ob/ob mice. The tissue specific expression of Slc25a12 and Slc25a13 together with the clear regulation in BAT and heart in the genetic model for obesity, suggests that Slc25a12 and Slc25a13 play a central role in the metabolism.

As shown in FIG. 16A, real time PCR (Taqman) analysis of the expression of the myelin gene expression factor 2 (MEF-2) RNA in mammalian (mouse) tissues revealed that MEF-2 is highly expressed in hypothalamus, brain and testis. Furthermore it shows robust expression levels in WAT, colon, lung, spleen and kidney. FIG. 16B shows that MEF-2 is upregulated ins BAT of ob/ob mice. FIG. 16C shows that MEF-2 is also upregulated in BAT after high fat (palmitate) diet feeding. The upregulation of MEF-2 in BAT of a genetic model of obesity as well as under high fat diet suggests a central role for MEF-2 in metabolism.

### EXAMPLE 7

### Analysis of the Differential Expression of Transcripts of the Proteins of the Invention in Human Tissues

RNA preparation from human primary adipose tissues was done as described in Example 6. The hybridization and scanning was performed as described in the manufacturer's manual (see Affymetrix Technical Manual, 2002, obtained from Affmetrix, Santa Clara, USA).

In FIGS. 12A and B, 20, 24, and 28, the X-axis represents the time axis, shown are day 0 and day 12 of adipocyte differentiation. The Y-axis represents the flourescent intensity. The expression analysis (using Affymetrix GeneChips) of the Quaking 6 (QKI6), RNA binding protein HQK-7B, RNA binding protein with multiple splicing (RBPMS), Peroxiredoxin 1 (PRDX1), and hypothetical protein LOC55565 genes using primary human abdominal adipocycte differentiation clearly shows differential expression of human QKI6, HQK-7B, RBPMS, PRDX1, and LOC55565 genes in adipocytes. Several independent experiments were done. The experiments show that the QKI6 (see FIG. 12A), HQK-7B (see FIG. 12B), and PRDX1 (see FIG. 24) are most abundant at day 12 compared to day 0 during differentiation. The experiments further show that the RBPMS (see FIG. 20) and LOC55565 (see FIG. 28) transcripts are most abundant at day 0 compared to day 12 during differentiation.

Thus, the QKI6, HQK-7B, or PRDX1 proteins have to be significantly increased in order for the preadipocyctes to differentiate into mature adipocycte. The QKI6, HQK-7B, or PRDX1 prroteins in preadipocyctes have the potential to enhance adipose differentiation at a very early stage. The RBPMS or LOC55565 proteins have to be significantly decreased in order for the preadipocyctes to differentiate into mature adipocycte. Therefore, the RBPMS or LOC55565 proteins in preadipocyctes have the potential to inhibit adipose differentiation at a very early stage.

Therefore, QKI6, HQK-7B, RBPMS, PRDX1, and LOC55565 proteins might play an essential role in the regulation of human metabolism, in particular in the regulation of adipogenesis and thus it might play an essential role in obesity, diabetes, and/or metabolic syndrome.

For the purpose of the present invention, it will understood by the person having average skill in the art that any combination of any feature mentioned throughout the specification is explicitly disclosed herewith.

1. A pharmaceutical composition comprising a CG7956, aralar1, how, CG9373, cpo, Jafrac1, or CG14440 nucleic acid molecule or a polypeptide encoded thereby and/or a functional fragment thereof or an effector/modulator of said nucleic acid molecule and/or a polypeptide encoded thereby, preferably together with pharmaceutically acceptable carriers, diluents and/or additives.

2. The composition of claim 1, wherein the nucleic acid molecule is a vertebrate or insect CG7956, aralar1, how, CG9373, cpo, Jafrac 1, or CG14440 nucleic acid, particulary encoding a human protein as described in Table 1, and/or a nucleic molecule which is complementary thereto, or a functional fragment thereof or a variant thereof.

3. The composition of claim 1, or wherein said nucleic acid molecule is selected from the group consisting of
(a) a nucleic acid molecule encoding a polypeptide as shown in Table 1;
(b) a nucleic acid molecule which comprises or is the nucleic acid molecule as shown in Table 1;
(c) a nucleic acid molecule degenerate as a result of the genetic code to the nucleic acid sequences as defined (a) or (b);
(d) a nucleic acid molecule that hybridizes at 50° C. in a solution containing 1×SSC and 0.1% SDS to a nucleic acid molecule as defined in claim 2 and/or a nucleic acid molecule which is complementary thereto;
(e) a nucleic acid molecule that encodes a polypeptide which is at least 85%, preferably at least 90%, more preferably at least 95%, more preferably at least 98% and up to 99.6% identical to a human protein as described in Table 1 or as defined in claim 2; and a nucleic acid molecule that differs from the nucleic acid molecule of (a) to (e) by mutation and wherein said mutation causes an alteration, deletion, duplication or premature stop in the encoded polypeptide.

4. The composition of claim 1, wherein the nucleic acid molecule is a DNA molecule, particularly a cDNA or a genomic DNA.

5. The composition of claim 1, wherein said nucleic acid encodes a polypeptide contributing to regulating the energy homeostasis and/or the metabolism of triglycerides.

6. The composition of claim 1, wherein said nucleic acid molecule is a recombinant nucleic acid molecule.

7. The composition of claim 1, wherein the nucleic acid molecule is a vector, particularly an expression vector.

8. The composition of claim 1, wherein the polypeptide is a recombinant polypeptide.

9. The composition of claim 8, wherein said recombinant polypeptide is a fusion polypeptide.

10. The composition of claim 1, wherein said nucleic acid molecule is selected from hybridization probes, primers and anti-sense oligonucleotides.

11. The composition of claim 1 which is a diagnostic composition.

12. The composition of claim 1 which is a therapeutic composition.

13. The composition of claim 1 for the manufacture of an agent for detecting and/or verifying, for the treatment, alleviation and/or prevention of metabolic diseases or dysfunctions, including metabolic syndrome, obesity, and/or diabetes, as well as related disorders such as eating disorder, cachexia, hypertension, coronary heart disease, hypercholesterolemia, dyslipidemia, osteoarthritis, or gallstones, in cells, cell masses, organs and/or subjects.

14. Use of a CG7956, aralar1, how, CG9373, cpo, Jafrac 1, or CG 14440 nucleic acid molecule, particularly of a nucleic acid molecule according to claim 3(a), (b) or (c), or a polypeptide is encoded thereby or a functional fragment or a variant of said nucleic acid molecule or said polypeptide and/or an effector/modulator of said nucleic or polypeptide for the manufacture of a medicament for the treatment of obesity, diabetes, and/or metabolic syndrome for controlling the function of a gene and/or a gene product which is influenced and/or modified by a CG7956, aralar1, how, CG9373, cpo, Jafrac 1, or CG14440 polypeptide, particularly by a polypeptide according to claim 3.

15. Use of a CG7956, aralar1, how, CG9373, cpo, Jafrac1, or CG14440 nucleic acid molecule, particularly of a nucleic acid molecule according to claim 3(a), (b) or (c), or a polypeptide encoded thereby or a functional fragment or a variant of said nucleic acid molecule or said polypeptide or use of an effector/modulator of said nucleic acid molecule or said polypeptide for identifying substances in vitro capable of interacting with a CG7956, aralar1, how, CG 9373, cpo, Jafrac 1, or CG 14440 polypeptide, particularly with a polypeptide according to claim 3.

16. A non-human transgenic animal exhibiting a modified expression of a CG7956, aralar1, how, CG9373, cpo, Jafrac1, or CG14440 polypeptide, particularly of a polypeptide according to claim 3.

17. The animal of claim 16, wherein the expression of the CG7956, aralar1, how, CG9373, cpo, Jafrac1, or CG14440 polypeptide, is increased and/or reduced.

18. A recombinant host cell exhibiting a modified expression of a CG7956, aralar1, how, CG9373, cpo, Jafrac1, or CG14440 polypeptide, particularly of a polypeptide according to claim 3.

19. The cell of claim 18 which is a human cell.

20. A method of identifying a (poly)peptide involved in the regulation of energy homeostasis and/or metabolism of triglycerides in a mammal comprising the steps of
(a) contacting a collection of (poly)peptides with a CG7956, aralar1, how, CG9373, cpo, Jafrac I, or CG 14440 polypeptide, particularly of a polypeptide according to claim 3, or a functional fragment thereof under conditions that allow binding of said (poly)peptides;
(b) removing (poly)peptides which do not bind and
(c) identifying (poly)peptides that bind to said polypeptide.

21. A method of screening for an agent which modulates/effects the interaction of a CG7956, aralar1, how, CG9373, cpo, Jafrac1, or CG 14440 polypeptide, particularly of a polypeptide according to claim 3, with a binding target, comprising the steps of (a) incubating a mixture comprising(aa) a CG7956, aralar1, how, CG9373, cpo, Jafrac1, or CG 14440 polypeptide, particularly of a polypeptide according to claim 3, or a functional fragment thereof;
(ab) a binding target/agent of said polypeptide or functional fragment thereof; and
(ac) a candidate agent under conditions whereby said polypeptide or functional fragment thereof specifically binds to said binding target/agent at a reference affinity;
(b) detecting the binding affinity of said polypeptide or functional fragment thereof to said binding target to determine an affinity for the agent; and
(c) determining a difference between affinity for the agent and the reference affinity.

22. A method for screening for an agent, which modulates/effects the activity of a CG7956, aralar1, how, CG9373, cpo, Jafrac1, or CG14440 polypeptide, particularly of a polypeptide according to claim 3, comprising the steps of
(a) incubating a mixture comprising
(aa) said polypeptide or a functional fragment thereof and
(ab) a candidate agent under conditions whereby said polypeptide or functional fragment thereof has a reference activity;
(b) detecting the activity of said polypeptide or functional fragment thereof to determine an activity in the presence of the agent; and
(c) determining a difference between the activity in the presence of the agent and the reference activity.

23. A method of producing a composition comprising mixing the (poly)peptide identified by the method of claim 20 with a pharmaceutically acceptable carrier, diluent and/or additive.

24. The method of claim 23 wherein said composition is a pharmaceutical composition for preventing, alleviating or treating of metabolic diseases or dysfunctions, including metabolic syndrome, obesity, and/or diabetes, as well as related disorders such as eating disorder, cachexia, hypertension, coronary heart disease, hypercholesterolemia, dyslipidemia, osteoarthritis, or gallstones.

25. Use of a (poly)peptide as identified by the method of claim 20 for the preparation of a pharmaceutical composition for the treatment, alleviation and/or prevention of metabolic diseases or dysfunctions, including metabolic syndrome, obesity, and/or diabetes, as well as related disorders such as eating disorder, cachexia, hypertension, coronary heart disease, hypercholesterolemia, dyslipidemia, osteoarthritis, or gallstones.

26. Use of a nucleic acid molecule as defined in claim 1 for the preparation of a medicament for the treatment, alleviation and/or prevention of metabolic diseases or dysfunctions, including obesity, diabetes, and/or metabolic syndrome, as well as related disorders such as eating disorder, cachexia, hypertension, coronary heart disease, hypercholesterolemia, dyslipidemia, osteoarthritis, or gallstones.

27. Use of a polypeptide as defined in claim 1 for the preparation of a medicament for the treatment, alleviation and/or prevention of metabolic diseases or dysfunctions, including obesity, diabetes, and/or metabolic syndrome, as well as related disorders such as eating disorder, cachexia, hypertension, coronary heart disease, hypercholesterolemia, dyslipidemia, osteoarthritis, or gallstones.

28. Use of a vector as defined in claim 7 for the preparation of a medicament for the treatment, alleviation and/or prevention of metabolic diseases or dysfunctions, including obesity, diabetes, and/or metabolic syndrome, as well as related disorders such as eating disorder, cachexia, hypertension, coronary heart disease, hypercholesterolemia, dyslipidemia, osteoarthritis, or gallstones.

29. Use of a host cell as defined in claim 18 for the preparation of a medicament for the treatment, alleviation and/or prevention of metabolic diseases or dysfunctions, including obesity, diabetes, and/or metabolic syndrome, as well as related disorders such as eating disorder, cachexia, hypertension, coronary heart disease, hypercholesterolemia, dyslipidemia, osteoarthritis, or gallstones.

30. Use of a CG7956, aralar1, how, CG9373, cpo, Jafrac 1, or CG 14440 nucleic acid molecule or of a functional fragment thereof for the production of a non-human transgenic animal which over- or under-expresses the CG7956, aralar1, how, CG9373, cpo, Jafrac 1, or CG 14440 gene product.

31. Kit comprising at least one of
(a) a CG7956, aralar1, how, CG9373, cpo, Jafrac 1, or CG 14440 nucleic acid molecule or a functional fragment thereof;
(b) a CG7956, aralar1, how, CG9373, cpo, Jafrac 1, or CG 14440 amino acid molecule or a functional fragment thereof;
(c) a vector comprising the nucleic acid of (a);
(d) a host cell comprising the nucleic acid of (a) or the vector of (c);
(e) a polypeptide encoded by the nucleic acid of (a);
(f) a fusion polypeptide encoded by the nucleic acid of (a);
(g) an antibody, an aptamer or another effector/modulator against the nucleic acid of (a) or the polypeptide of (b), (e) or (f) and
(h) an anti-sense oligonucleotide of the nucleic acid of (a).

32. A method of producing a composition comprising the agent identified by the method of claim 21 with a pharmaceutically acceptable carrier, diluent and/or additive.

33. Use of an agent as identified by the method of claim 21 for the preparation of a pharmaceutical composition for the treatment, alleviation and/or prevention of metabolic diseases or dysfunctions, including metabolic syndrome, obesity, and/or diabetes, as well as related disorders such as eating disorder, cachexia, hypertension, coronary heart disease, hypercholesterolemia, dyslipidemia, osteoarthritis, or gallstones.


SEQUENCE LISTING (73 sequences)

SEQ ID NO: 1 - protein, 615 aa, Drosophila melanogaster
LVKIANYQDLLGSHHQLLIAATAAAAAAAAAEPQLQLQHLLPAAPTTPAVISNPINSIGPINQISSSSHPSNNNQQAVFEKAITISSIAIKRRPTLPQTPASAPQVLSPSPKRQCAAAVSVLPVTVPVPVPVSVPLPVSVPVPVSVKGHPISHTHQIAHTHQISHSHPISHPHHHQLSFAHPTQFAAAVAAHHQQQQQQQAQQQQQAVQQQQQQAVQQQQVAYAVAASPQLQQQQQQQQHRLAQFNQAAAAALLNQHLQQQHQAQQQQHQAQQQSLAHYGGYQLHRYAPQQQQQHILLSSGSSSSKHNSNNNSNTSAGAASAAVPIATSVAAVPTTGGSLPDSPAHESHSHESNSATASAPTTPSPAGSVTSAAPTATATAAAAGSAAATAAATGTPATSAVSDSNNNLNSSSSSNSNSNAIMENQMALAPLGLSQSMDSVNTASNEEEVRTLFVSGLPMDAKPRELYLLFRAYEGYEGSLLKVTSKNGKTASPVGFVTFHTRAGAEAAKQDLQGVRFDPDMPQTIRLEFAKSNTKVSKPKPQPNTATTASHPALMHPLTGHLGGPFFPGGPELWHHPLAYSAAAAAELPGAAALQHATLVHPALHPQVPVRSYL

SEQ ID NO: 2 - DNA, 17 bp, Mus musculus
cctggatcgcaccaacg

SEQ ID NO: 3 - DNA, 22 bp, Mus musculus
ttaagctgctgttccatgacca

SEQ ID NO: 4 - DNA, 20 bp, Mus musculus
  misc_feature (1)..(1): FAM reporter dye
tccaggctgccatagcgcgc

SEQ ID NO: 5 - DNA, 19 bp, Mus musculus
cctgccaaccctgatcaca

SEQ ID NO: 6 - DNA, 20 bp, Mus musculus
tttcaatgccagcgaaagtg

SEQ ID NO: 7 - DNA, 23 bp, Mus musculus
  misc_feature (1)..(1): FAM reporter dye
cggtggctacagacttgccacgg

SEQ ID NO: 8 - DNA, 22 bp, Mus musculus
agcggtggttctatgtcgattt

SEQ ID NO: 9 - DNA, 19 bp, Mus musculus
cgggatttaggaaccggct

SEQ ID NO: 10 - DNA, 22 bp, Mus musculus
  misc_feature (1)..(1): FAM reporter dye
aggcgtgaagcccgtgggatct

SEQ ID NO: 11 - DNA, 21 bp, Mus musculus
acaaggatggcaagagcagag

SEQ ID NO: 12 - DNA, 22 bp, Mus musculus
atggaaattgcttggactgctt

SEQ ID NO: 13 - DNA, 26 bp, Mus musculus
  misc_feature (1)..(1): FAM reporter dye
catgggcactgtcacttttgagcagg

SEQ ID NO: 14 - protein, 914 aa, Drosophila melanogaster
MEVFQTDSHYIFVKRDKSLWWHRRTSEFSIKAGWDLSSVDDIECIGVTHGIVGVISLPNVYEPHLVVVKEASAVGVLYPPHLVYKIKSICILSADDPDTDLPNCTKHTKSNQSTPTHSVSTSNNNNASVPSSGGGSSKSTKLFEGMNKTWGAVKSAGNTIKNTTQQAANLATKQVKSSVGIREPRHIERRITEELHKIFDETDSFYFSFDCDITNNLQRHEAKSEESQSQPDERFFWNKHMIRDLINLNDKTWILPIIQGFMQVENCVIGNECFTLALVSRRSRHRAGTRYKRRGVDEKGNCANYVETEQILSFRHHQLSFTQVRGSVPIYWSQPGYKYRPPPRLDRGVAETQQAFELHFTKELETYGRVCIVNLVEQSGKEKTIGDAYADHVIKLNNDRLIYVTFDFHDYCRGMRFENVSALIDAVGPEAGAMGFHWQDQRGMICNQKSVFRVNCMDCLDRTNVVQTAIGKAVLESQLVKLGLSPPYTPIPEQLKSPFMVLWANNGDIISRQYAGTNALKGDYTRTGERKISGMMKDGMNSANRYYLARFKDSYRQATIDLMLGNQVSSESLSALGGQAGPDENDGTENAEQAKLLVEDCRRLLLGTAQYPVGAWGLIDADPSSGDINETEVDTILLLTDDCYIVAEYDSHLDKIVRFEKVQLTQVRLIELGMHQQTKIFQGSAPAHLCLRLNYSVDEQEGYFHMFRSANLRFFNNMAYVIKTQEEVAESMTSIVEMFRIALDNAGNTEVRYITGGVLQRRKSKLPTLDVPRGMPRNLSESQLVQLSSKALSNMAGQFSKLGQTFKKPQAHPSSLAATMNPQVMRQRDSEIESGQEAEKAVFTLGRKHRNSNSASSTDTDEHDNSLYEPEVDSDVEIAMDKSNYNENAFLPSVGIVMGNQKEDSPSSSDEIRH

SEQ ID NO: 15 - protein, 916 aa, Homo sapiens
MELFQAKDHYILQQGERALWCSRRDGGLQLRPATDLLLAWNPICLGLVEGVIGKIQLHSDLPWWLILIRQKALVGKLPGDHEVCKVTKIAVLSLSEMEPQDLELELCKKHHFGINKPEKIIPSPDDSKFLLKTFTHIKSNVSAPNKKKVKESKEKEKLERRLLEELLKMFMDSESFYYSLTYDLTNSVQRQSTGERDGRPLWQKVDDRFFWNKYMIQDLTEIGTPDVDFWIIPMIQGFVQIEELVVNYTESSDDEKSSPETPPQESTCVDDIHPRFLVALISRRSRHRAGMRYKRRGVDKNGNVANYVETEQLIHVHNHTLSFVQTRGSVPVFWSQVGYRYNPRPRLDRSEKETVAYFCAHFEEQLNIYKKQVIINLVDQAGREKIIGDAYLKQVLLFNNSHLTYVSFDFHEHCRGMKFENVQTLTDAIYDIILDMKWCWVDEAGVICKQEGIFRVNCMDCLDRTNVVQAAIARVVMEQQLKKLGVMPPEQPLPVKCNRIYQIMWANNGDSISRQYAGTAALKGDFTRTGERKLAGVMKDGVNSANRYYLNRFKDAYRQAVIDLMQGIPVTEDLYSIFTKEKEHEALHKENQRSHQELISQLLQSYMKLLLPDDEKFHGGWALIDCDPSLIDATHRDVDVLLLLSNSAYYVAYYDDEVDKVNQYQRLSLENLEKIEIGPEPTLFGKPKFSCMRLHYRYKEASGYFHTLRAVMRNPEEDGKDTLQCIAEMLQITKQAMGSDLPIIEKKLERKSSKPHEDIIGIRSQNQGSLAQGKNFLMSKFSSLNQKVKQTKSNVNIGNLRKLGNFTKPEMKVNFLKPNLKVNLWKSDSSLETMENTGVMDKVQAESDGDMSSDNDSYHSDEFLTNSKSDEDRQLANSLESVGPIDYVLPSCGIIASAPRLGSRSQSLSSTDSSVH

SEQ ID NO: 16 - protein, 643 aa, Drosophila melanogaster
MTSEDFVRKFLGLFSESAFNDESVRLLANIADTSKDGLISFSEFQAFEGLLCTPDALYRTAFQLFDRKGNGTVSYADFADVVQKTELHSKIPFSLDGPFIKRYFGDKKQRLINYAEFTQLLHDFHEEHAMEAFRSKDPAGTGFISPLDFQDIIVNVKRHLLTPGVRDNLVSVTEGHKVSFPYFIAFTSLLNNMELIKQVYLHATEGSRTDMITKDQILLAAQTMSQITPLEIDILFHLAGAVHQAGRIDYSDLSNIAPEHYTKHMTHRLAEIKAVESPADRSAFIQVLESSYRFTLGSFAGAVGATVVYPIDLVKTRMQNQRAGSYIGEVAYRNSWDCFKKVVRHEGFMGLYRGLLPQLMGVAPEKAIKLTVNDLVRDKLTDKKGNIPTWAEVLAGGCAGASQVVFTNPLEIVKIRLQVAGEIASGSKIRAWSVVRELGLFGLYKGARACLLRDVPFSAIYFPTYAHTKAMMADKDGYNHPLTLLAAGAIAGVPAASLVTPADVIKTRLQVVARSGQTTYTGVWDATKKIMAEEGPRAFWKGTAARVFRSSPQFGVTLVTYELLQRLFYVDFGGTQPKGSEAHKITTPLEQAAASVTTENVDHIGGYRAAVPLLAGVESKFGLYLPRFGRGVTAASPSTATGS

SEQ ID NO: 17 - protein, 643 aa, Homo sapiens
MTPEDFVQRYLGLYNDPNSNPKIVQLLAGVADQTKDGLISYQEFLAFESVLCAPDSMFIVAFQLFDKSGNGEVTFENVKEIFGQTIIHHHIPFNWDCEFIRLHFGHNRKKHLNYTEFTQFLQELQLEHARQAFALKDKSKSGMISGLDFSDIMVTIRSHMLTPFVEENLVSAAGGSISHQVSFSYFNAFNSLLNNMELVRKIYSTLAGTRKDVEVTKEEFAQSAIRYGQVTPLEIDILYQLADLYNASGRLTLADIERIAPLAEGALPYNLAELQRQQSPGLGRPIWLQIAESAYRFTLGSVAGAVGATAVYPIDLVKTRMQNQRGSGSVVGELMYKNSFDCFKKVLRYEGFFGLYRGLIPQLIGVAPEKAIKLTVNDFVRDKFTRRDGSVPLPAEVLAGGCAGGSQVIFTNPLEIVKIRLQVAGEITTGPRVSALNVLRDLGIFGLYKGAKACFLRDIPFSAIYFPVYAHCKLLLADENGHVGGLNLLAAGAMAGVPAASLVTPADVIKTRLQVAARAGQTTYSGVIDCFRKILREEGPSAFWKGTAARVFRSSPQFGVTLVTYELLQRWFYIDFGGLKPAGSEPTPKSRIADLPPANPDHIGGYRLATATFAGIENKFGLYLPKFKSPSVAVVQPKAAVAA

SEQ ID NO: 18 - protein, 635 aa, Drosophila melanogaster
MTSEDFVRKFLGLFSESAFNDESVRLLANIADTSKDGLISFSEFQAFEGLLCTPDALYRTAFQLFDRKGNGTVSYADFADVVQKTELHSKIPFSLDGPFIKRYFGDKKQRLINYAEFTQLLHDFHEEHAMEAFRSKDPAGTGFISPLDFQDIIVNVKRHLLTPGVRDNLVSVTEGHKVSFPYFIAFTSLLNNMELIKQVYLHATEGSRTDMITKDQILLAAQTMSQITPLEIDILFHLAGAVHQAGRIDYSDLSNIAPEHYTKHMTHRLAEIKAVESPADRSAFIQVLESSYRFTLGSFAGAVGATVVYPIDLVKTRMQNQRAGSYIGEVAYRNSWDCFKKVVRHEGFMGLYRGLLPQLMGVAPEKAIKLTVNDLVRDKLTDKKGNIPTWAEVLAGGCAGASQVVFTNPLEIVKIRLQVAGEIASGSKIRAWSVVRELGLFGLYKGARACLLRDVPFSAIYFPTYAHTKAMMADKDGYNHPLTLLAAGAIAGVPAASLVTPADVIKTRLQVVARSGQTTYTGVWDATKKIMAEEGPRAFWKGTAARVFRSSPQFGVTLVTYELLQRLFYVDFGGTQPKGSEAHKITTPLEQAAASVTTENVDHIGGYRAAVPLLAGVESKFGLYLPRFGRGVTAA

SEQ ID NO: 19 - protein, 632 aa, Homo sapiens
MSPNDFVTRYLNIFGESQPNPKTVELLSGVVDQTKDGLISFQEFVAFESVLCAPDALFMVAFQLFDKAGKGEVTFEDVKQVFGQTTIHQHIPFNWDSEFVQLHFGKERKRHLTYAEFTQFLLEIQLEHAKQAFVQRDNARTGRVTAIDFRDIMVTIRPHVLTPFVEECLVAAAGGTTSHQVSFSYFNGFNSLLNNMELIRKIYSTLAGTRKDVEVTKEEFVLAAQKFGQVTPMEVDILFQLADLYEPRGRMTLADIERIAPLEEGTLPFNLAEAQRQKASGDSARPVLLQVAESAYRFGLGSVAGAVGATAVYPIDLVKTRMQNQRSTGSFVGELMYKNSFDCFKKVLRYEGFFGLYRGLLPQLLGVAPEKAIKLTVNDFVRDKFMHKDGSVPLAAEILAGGCAGGSQVIFTNPLEIVKIRLQVAGEITTGPRVSALSVVRDLGFFGIYKGAKACFLRDIPFSAIYFPCYAHVKASFANEDGQVSPGSLLLAGAIAGMPAASLVTPADVIKTRLQVAARAGQTTYSGVIDCFRKILREEGPKALWKGAGARVFRSSPQFGVTLLTYELLQRWFYIDFGGVKPMGSEPVPKSRINLPAPNPDHVGGYKLAVATFAGIENKFGLYLPLFKPSVS

SEQ ID NO: 20 - protein, 682 aa, Drosophila melanogaster
MPLTKSLPNSPSLLKRAGTEKLREVFLKYASIQKNGEHYMTSEDFVRKFLGLFSESAFNDESVRLLANIADTSKDGLISFSEFQAFEGLLCTPDALYRTAFQLFDRKGNGTVSYADFADVVQKTELHSKIPFSLDGPFIKRYFGDKKQRLINYAEFTQLLHDFHEEHAMEAFRSKDPAGTGFISPLDFQDIIVNVKRHLLTPGVRDNLVSVTEGHKVSFPYFIAFTSLLNNMELIKQVYLHATEGSRTDMITKDQILLAAQTMSQITPLEIDILFHLAGAVHQAGRIDYSDLSNIAPEHYTKHMTHRLAEIKAVESPADRSAFIQVLESSYRFTLGSFAGAVGATVVYPIDLVKTRMQNQRAGSYIGEVAYRNSWDCFKKVVRHEGFMGLYRGLLPQLMGVAPEKAIKLTVNDLVRDKLTDKKGNIPTWAEVLAGGCAGASQVVFTNPLEIVKIRLQVAGEIASGSKIRAWSVVRELGLFGLYKGARACLLRDVPFSAIYFPTYAHTKAMMADKDGYNHPLTLLAAGAIAGVPAASLVTPADVIKTRLQVVARSGQTTYTGVWDATKKIMAEEGPRAFWKGTAARVFRSSPQFGVTLVTYELLQRLFYVDFGGTQPKGSEAHKITTPLEQAAASVTTENVDHIGGYRAAVPLLAGVESKFGLYLPRFGRGVTAASPSTATGS

SEQ ID NO: 21 - protein, 678 aa, Homo sapiens
MAVKVQTTKRGDPHELRNIFLQYASTEVDGERYMTPEDFVQRYLGLYNDPNSNPKIVQLLAGVADQTKDGLISYQEFLAFESVLCAPDSMFIVAFQLFDKSGNGEVTFENVKEIFGQTIIHHHIPFNWDCEFIRLHFGHNRKKHLNYTEFTQFLQELQLEHARQAFALKDKSKSGMISGLDFSDIMVTIRSHMLTPFVEENLVSAAGGSISHQVSFSYFNAFNSLLNNMELVRKIYSTLAGTRKDVEVTKEEFAQSAIRYGQVTPLEIDILYQLADLYNASGRLTLADIERIAPLAEGALPYNLAELQRQQSPGLGRPIWLQIAESAYRFTLGSVAGAVGATAVYPIDLVKTRMQNQRGSGSVVGELMYKNSFDCFKKVLRYEGFFGLYRGLIPQLIGVAPEKAIKLTVNDFVRDKFTRRDGSVPLPAEVLAGGCAGGSQVIFTNPLEIVKIRLQVAGEITTGPRVSALNVLRDLGIFGLYKGAKACFLRDIPFSAIYFPVYAHCKLLLADENGHVGGLNLLAAGAMAGVPAASLVTPADVIKTRLQVAARAGQTTYSGVIDCFRKILREEGPSAFWKGTAARVFRSSPQFGVTLVTYELLQRWFYIDFGGLKPAGSEPTPKSRIADLPPANPDHIGGYRLATATFAGIENKFGLYLPKFKSPSVAVVQPKAAVAATQ

SEQ ID NO: 22 - protein, 675 aa, Homo sapiens
MAAAKVALTKRADPAELRTIFLKYASIEKNGEFFMSPNDFVTRYLNIFGESQPNPKTVELLSGVVDQTKDGLISFQEFVAFESVLCAPDALFMVAFQLFDKAGKGEVTFEDVKQVFGQTTIHQHIPFNWDSEFVQLHFGKERKRHLTYAEFTQFLLEIQLEHAKQAFVQRDNARTGRVTAIDFRDIMVTIRPHVLTPFVEECLVAAAGGTTSHQVSFSYFNGFNSLLNNMELIRKIYSTLAGTRKDVEVTKEEFVLAAQKFGQVTPMEVDILFQLADLYEPRGRMTLADIERIAPLEEGTLPFNLAEAQRQKASGDSARPVLLQVAESAYRFGLGSVAGAVGATAVYPIDLVKTRMQNQRSTGSFVGELMYKNSFDCFKKVLRYEGFFGLYRGLLPQLLGVAPEKAIKLTVNDFVRDKFMHKDGSVPLAAEILAGGCAGGSQVIFTNPLEIVKIRLQVAGEITTGPRVSALSVVRDLGFFGIYKGAKACFLRDIPFSAIYFPCYAHVKASFANEDGQVSPGSLLLAGAIAGMPAASLVTPADVIKTRLQVAARAGQTTYSGVIDCFRKILREEGPKALWKGAGARVFRSSPQFGVTLLTYELLQRWFYIDFGGVKPMGSEPVPKSRINLPAPNPDHVGGYKLAVATFAGIENKFGLYLPLFKPSVSTSKAIGGGP

SEQ ID NO: 23 - protein, 323 aa, Drosophila melanogaster
QQQQSTQSIADYLAQLLKDRKQLAAFPNVFTHVERLLDEEIARVRASLFQINGVKKEPLTLPEPEGSVVTMNEKVYVPVREHPDFNFVGRILGPRGMTAKQLEQETGCKIMVRGKGSMRDKKKEDANRGKPNWEHLSDDLHVLITVEDTENRATVKLAQAVAEVQKLLVPQAEGEDELKKRQLMELAIINGTYRDTTAKSVAVCDEEWRRLVAASDSRLLTSTGLPGLAAQIRAPAAAPLGAPLILNPRMTVPTTAASILSAQAAPTAAFDQTGHGMIFAPYDYANYAALAGNPLLTEYADHSVGAIKQQRRLATNREHPYQR

SEQ ID NO: 24 - protein, 325 aa, Homo sapiens
ETKEKPKPTPDYLMQLMNDKKLMSSLPNFCGIFNHLERLLDEEISRVRKDMYNDTLNGSTEKRSAELPDAVGPIVQLQEKLYVPVKEYPDFNFVGRILGPRGLTAKQLEAETGCKIMVRGKGSMRDKKKEEQNRGKPNWEHLNEDLHVLITVEDAQNRAEIKLKRAVEEVKKLLVPAAEGEDSLKKMQLMELAILNGTYRDANIKSPALAFSLAATAQAAPRIITGPAPVLPPAALRTPTPAGPTIMPLIRQIQTAVMPNGTPHPTAAIVPPGPEAGLIYTPYEYPYTLAPATSILEYPIEPSGVLGAVATKVRRHDMRVHPYQR

SEQ ID NO: 25 - protein, 325 aa, Homo sapiens; identical to SEQ ID NO: 24

SEQ ID NO: 26 - protein, 325 aa, Homo sapiens; identical to SEQ ID NO: 24

SEQ ID NO: 27 - protein, 309 aa, Drosophila melanogaster
QLLKDRKQLAAFPNVFTHVERLLDEEIARVRASLFQINGVKKEPLTLPEPEGSVVTMNEKVYVPVREHPDFNFVGRILGPRGMTAKQLEQETGCKIMVRGKGSMRDKKKEDANRGKPNWEHLSDDLHVLITVEDTENRATVKLAQAVAEVQKLLVPQAEGEDELKKRQLMELAIINGTYRDTTAKSVAVCDEEWRRLVAASDSRLLTSTGLPGLAAQIRAPAAAPLGAPLILNPRMTVPTTAASILSAQAAPTAAFDQTGHGMIFAPYDYANYAALAGNPLLTEYADHSVGAIKQQRRLATNREHPYQR

SEQ ID NO: 28 - protein, 311 aa, Homo sapiens
QLMNDKKLMSSLPNFCGIFNHLERLLDEEISRVRKDMYNDTLNGSTEKRSAELPDAVGPIVQLQEKLYVPVKEYPDFNFVGRILGPRGLTAKQLEAETGCKIMVRGKGSMRDKKKEEQNRGKPNWEHLNEDLHVLITVEDAQNRAEIKLKRAVEEVKKLLVPAAEGEDSLKKMQLMELAILNGTYRDANIKSPALAFSLAATAQAAPRIITGPAPVLPPAALRTPTPAGPTIMPLIRQIQTAVMPNGTPHPTAAIVPPGPEAGLIYTPYEYPYTLAPATSILEYPIEPSGVLGAVATKVRRHDMRVHPYQR

SEQ ID NO: 29 - protein, 284 aa, Drosophila melanogaster
QQQQSTQSIADYLAQLLKDRKQLAAFPNVFTHVERLLDEEIARVRASLFQINGVKKEPLTLPEPEGSVVTMNEKVYVPVREHPDFNFVGRILGPRGMTAKQLEQETGCKIMVRGKGSMRDKKKEDANRGKPNWEHLSDDLHVLITVEDTENRATVKLAQAVAEVQKLLVPQAEGEDELKKRQLMELAIINGTYRDTTAKSVAVCDEEWRRLVAASDSRLLTSTGLPGLAAQIRAPAAAPLGAPLILNPRMTVPTTAASILSAQAAPTAAFDQTGHGMIFAPYDY

SEQ ID NO: 30 - protein, 285 aa, Homo sapiens
ETKEKPKPTPDYLMQLMNDKKLMSSLPNFCGIFNHLERLLDEEISRVRKDMYNDTLNGSTEKRSAELPDAVGPIVQLQEKLYVPVKEYPDFNFVGRILGPRGLTAKQLEAETGCKIMVRGKGSMRDKKKEEQNRGKPNWEHLNEDLHVLITVEDAQNRAEIKLKRAVEEVKKLLVPAAEGEDSLKKMQLMELAILNGTYRDANIKSPALAFSLAATAQAAPRIITGPAPVLPPAALRTPTPAGPTIMPLIRQIQTAVMPNGTPHPTAAIVPPGPEAGLIYTPYEY

SEQ ID NO: 31 - protein, 285 aa, Homo sapiens; identical to SEQ ID NO: 30

SEQ ID NO: 32 - protein, 285 aa, Homo sapiens; identical to SEQ ID NO: 30

SEQ ID NO: 33 - protein, 285 aa, Homo sapiens; identical to SEQ ID NO: 30

SEQ ID NO: 34 - protein, 285 aa, Homo sapiens; identical to SEQ ID NO: 30

SEQ ID NO: 35 - protein, 285 aa, Homo sapiens; identical to SEQ ID NO: 30

SEQ ID NO: 36 - protein, 285 aa, Homo sapiens; identical to SEQ ID NO: 30

SEQ ID NO: 37 - protein, 313 aa, Drosophila melanogaster
DYLAQLLKDRKQLAAFPNVFTHVERLLDEEIARVRASLFQINGVKKEPLTLPEPEGSVVTMNEKVYVPVREHPDFNFVGRILGPRGMTAKQLEQETGCKIMVRGKGSMRDKKKEDANRGKPNWEHLSDDLHVLITVEDTENRATVKLAQAVAEVQKLLVPQAEGEDELKKRQLMELAIINGTYRDTTAKSVAVCDEEWRRLVAASDSRLLTSTGLPGLAAQIRAPAAAPLGAPLILNPRMTVPTTAASILSAQAAPTAAFDQTGHGMIFAPYDYANYAALAGNPLLTEYADHSVGAIKQQRRLATNREHPYQR

SEQ ID NO: 38 - protein, 315 aa, Homo sapiens
DYLMQLMNDKKLMSSLPNFCGIFNHLERLLDEEISRVRKDMYNDTLNGSTEKRSAELPDAVGPIVQLQEKLYVPVKEYPDFNFVGRILGPRGLTAKQLEAETGCKIMVRGKGSMRDKKKEEQNRGKPNWEHLNEDLHVLITVEDAQNRAEIKLKRAVEEVKKLLVPAAEGEDSLKKMQLMELAILNGTYRDANIKSPALAFSLAATAQAAPRIITGPAPVLPPAALRTPTPAGPTIMPLIRQIQTAVMPNGTPHPTAAIVPPGPEAGLIYTPYEYPYTLAPATSILEYPIEPSGVLGAVATKVRRHDMRVHPYQR

SEQ ID NO: 39 - protein, 405 aa, Drosophila melanogaster
MSVCESKAVVQQQLQQHLQQQAAAAVVAVAQQQQAQAQAQAQAQAQQQQQAPQVVVPMTPQHLTPQQQQQSTQSIADYLAQLLKDRKQLAAFPNVFTHVERLLDEEIARVRASLFQINGVKKEPLTLPEPEGSVVTMNEKVYVPVREHPDFNFVGRILGPRGMTAKQLEQETGCKIMVRGKGSMRDKKKEDANRGKPNWEHLSDDLHVLITVEDTENRATVKLAQAVAEVQKLLVPQAEGEDELKKRQLMELAIINGTYRDTTAKSVAVCDEEWRRLVAASDSRLLTSTGLPGLAAQIRAPAAAPLGAPLILNPRMTVPTTAASILSAQAAPTAAFDQTGHGMIFAPYDYANYAALAGNPLLTEYADHSVGAIKQQRRLATNREHPYQRATVGVPAKPAGFIEIQ

SEQ ID NO: 40 - protein, 363 aa, Homo sapiens [sequence omitted]

SEQ ID NO: 41 - protein, 347 aa, Homo sapiens [sequence omitted]

SEQ ID NO: 42 - protein, 341 aa, Homo sapiens [sequence omitted]

SEQ ID NO: 43 - protein, 319 aa, Homo sapiens
MVGEMETKEKPKPTPDYLMQLMNDKKLMSSLPNFCGIFNHLERLLDEEISRVRKDMYNDTLNGSTEKRSAELPDAVGPIVQLQEKLYVPVKEYPDFNFVGRILGPRGLTAKQLEAETGCKIMVRGKGSMRDKKKEEQNRGKPNWEHLNEDLHVLITVEDAQNRAEIKLKRAVEEVKKLLVPAAEGEDSLKKMQLMELAILNGTYRDANIKSPALAFSLAATAQAAPRIITGPAPVLPPAALRTPTPAGPTIMPLIRQIQTAVMPNGTPHPTAAIVPPGPEAGLIYTPYEYPYTLAPATSILEYPIEPSGVLGKFFSPWG

SEQ ID NO: 44 - protein, 629 aa, Drosophila melanogaster
MSMDASNSVESREKERDRRGRGARGSRFTDADGNGNGAGSQGGGVAARDRSRERRNCRVYISNIPYDYRWQDLKDLFRRIVGSIEYVQLFFDESGKARGCGIVEFKDPENVQKALEKMNRYEVNGRELVVKEDHGEQRDQYGRIVRDGGGGGGGGGGVQGGNGGNNGGGGGGGRDHMDDRDRGFSRRDDDRLSGRNNFNMMSNDYNNSSNYNLYGLSASFLESLGISGPLHNKVFVANLDYKVDNKKLKQVFKLAGKVQSVDLSLDKEGNSRGFAVIEYDHPVEAVQAISMLDRQMLFDRRMTVRLDRIPDKNEGIKLPEGLGGVGIGLGPNGEPLRDVAHNLPNGGQSQGQLLGNAQQGSQLGSVGSQPNSSAVSNATTNLLNNLTGVMFGNHAAVQPSPVAPVQKPSLGNNTGSGGLNLNNLNPSILAAVVGNLGNQGGNLSNPLLSSSLSNLGLNLGNSGNDDNLPPSNVGLSNNYSSGGTGGGNSYSSGNNYSGGGGSSNLGYNAYSSSGGMGGGNGGVGVDGNDYNTGNPLDVYGGGSNVGNSNVGSANAVGASRKSDTIIIKNVPITCTWQTLRDKFREIGDVKFAEIRGNDVGVVRFFKERDAELAIALMDGSRLDGRNIKV

SEQ ID NO: 45 - protein, 543 aa, Homo sapiens
VKMENDESAKEEKSDLKEKSTGSKKANRFHPYSKDKNSGTGEKKGPNRNRVFISNIPYDMKWQAIKDLMREKVGEVTYVELFKDAEGKSRGCGVVEFKDEEFVKKALETMNKYDLSGRPLNIKEDPDGENARRALQRTGGSFPGGHVPDMGSGLMNLPPSILNNPNIPPEVISNLQAGRLGSTIFVANLDFKVGWKKLKEVFSIAGTVKRADIKEDKDGKSRGMGTVTFEQAIEAVQAISMFNGQFLFDRPMHVKMDDKSVPHEEYRSHDGKTPQLPRGLGGIGMGLGPGGQPISASQLNIGGVMGNLGPGGMGMDGPGFGGMNRIGGGIGFGGLEAMNSMGGFGGVGRMGELYRGAMTSSMERDFGRGDIGINRGFGDSFGRLGSAMIGGFAGRIGSSNMGPVGSGISGGMGSMNSVTGGMGMGLDRMSSSFDRMGPGIGAILERSIDMDRGFLSGPMGSGMRERIGSKGNQIFVRNLPFDLTWQKLKEKFSQCGHVMFAEIKMENGKSKGCGTVRFDSPESAEKACRIMNGIKISGREIDV

SEQ ID NO: 46 - protein, 114 aa, Drosophila melanogaster
GRGARGSRFTDADGNGNGAGSQGGGVAARDRSRERRNCRVYISNIPYDYRWQDLKDLFRRIVGSIEYVQLFFDESGKARGCGIVEFKDPENVQKALEKMNRYEVNGRELVVKED

SEQ ID NO: 47 - protein, 108 aa, Homo sapiens
GIGAILERSIDMDRGFLSGPMGSGMRERIGSKGNQIFVRNLPFDLTWQKLKEKFSQCGHVMFAEIKMENGKSKGCGTVRFDSPESAEKACRIMNGIKISGREIDVRLD

SEQ ID NO: 48 - protein, 170 aa, Drosophila melanogaster
DQYGRIVRDGGGGGGGGGGVQGGNGGNNGGGGGGGRDHMDDRDRGFSRRDDDRLSGRNNFNMMSNDYNNSSNYNLYGLSASFLESLGISGPLHNKVFVANLDYKVDNKKLKQVFKLAGKVQSVDLSLDKEGNSRGFAVIEYDHPVEAVQAISMLDRQMLFDRRMTVRLDR

SEQ ID NO: 49 - protein, 169 aa, Homo sapiens
DSFGRLGSAMIGGFAGRIGSSNMGPVGSGISGGMGSMNSVTGGMGMGLDRMSSSFDRMGPGIGAILERSIDMDRGFLSGPMGSGMRERIGSKGNQIFVRNLPFDLTWQKLKEKFSQCGHVMFAEIKMENGKSKGCGTVRFDSPESAEKACRIMNGIKISGREIDVRLDR

SEQ ID NO: 50 - protein, 519 aa, Homo sapiens
VKMENDESAKEEKSDLKEKSTGSKKANRFHPYSKDKNSGTGEKKGPNRNRVFISNIPYDMKWQAIKDLMREKVGEVTYVELFKDAEGKSRGCGVVEFKDEEFVKKALETMNKYDLSGRPLNIKEDPDGENARRASQRTGGSFPGGHVPDMGSGLMNLPPSILNNPNIPPEVISNLQAGRLGSTIFVANLDFKVGWKKLKEVFSIAGTVKRADIKEDKDGKSRGMGTVTFEQAIEAVQAISMFNGQFLFDRPMHVKMDDKSVPHEEYRSHDGKTPQLPRGLGGIGMGLGPGGQPISASQLNIGGVMGNLGPGGMGMDGPGFGGMNRIGGGIGFGGLEAMNSMGGFGGVGRMGELYRGAMTSSMERDFGRGDIGINRGFGDSFGRLGGGMGGMNSVTGGMGMGLDRMSSSFDRMGPGIGAILERSIDMDRGFLSGPMGSGMRERIGSKGNQIFVRNLPFDLTWQKLKEKFSQCGHVMFAEIKMENGKSKGCGTVRFDSPESAEKACRIMNGIKISGREIDV

SEQ ID NO: 51 - protein, 255 aa, Drosophila melanogaster
RRNCRVYISNIPYDYRWQDLKDLFRRIVGSIEYVQLFFDESGKARGCGIVEFKDPENVQKALEKMNRYEVNGRELVVKEDHGEQRDQYGRIVRDGGGGGGGGGGVQGGNGGNNGGGGGGGRDHMDDRDRGFSRRDDDRLSGRNNFNMMSNDYNNSSNYNLYGLSASFLESLGISGPLHNKVFVANLDYKVDNKKLKQVFKLAGKVQSVDLSLDKEGNSRGFAVIEYDHPVEAVQAISMLDRQMLFDRRMTVRLDR

SEQ ID NO: 52 - protein, 345 aa, Homo sapiens
RLGSTIFVANLDFKVGWKKLKEVFSIAGTVKRADIKEDKDGKSRGMGTVTFEQAIEAVQAISMFNGQFLFDRPMHVKMDDKSVPHEEYRSHDGKTPQLPRGLGGIGMGLGPGGQPISASQLNIGGVMGNLGPGGMGMDGPGFGGMNRIGGGIGFGGLEAMNSMGGFGGVGRMGELYRGAMTSSMERDFGRGDIGINRGFGDSFGRLGGGMGGMNSVTGGMGMGLDRMSSSFDRMGPGIGAILERSIDMDRGFLSGPMGSGMRERIGSKGNQIFVRNLPFDLTWQKLKEKFSQCGHVMFAEIKMENGKSKGCGTVRFDSPESAEKACRIMNGIKISGREIDVRLDR

SEQ ID NO: 53 - protein, 108 aa, Homo sapiens; identical to SEQ ID NO: 47

SEQ ID NO: 54 - protein, 627 aa, Drosophila melanogaster
MDASNSVESREKERDRRGRGARGSRFTDADGNGNGAGSQGGGVAARDRSRERRNCRVYISNIPYDYRWQDLKDLFRRIVGSIEYVQLFFDESGKARGCGIVEFKDPENVQKALEKMNRYEVNGRELVVKEDHGEQRDQYGRIVRDGGGGGGGGGGVQGGNGGNNGGGGGGGRDHMDDRDRGFSRRDDDRLSGRNNFNMMSNDYNNSSNYNLYGLSASFLESLGISGPLHNKVFVANLDYKVDNKKLKQVFKLAGKVQSVDLSLDKEGNSRGFAVIEYDHPVEAVQAISMLDRQMLFDRRMTVRLDRIPDKNEGIKLPEGLGGVGIGLGPNGEPLRDVAHNLPNGGQSQGQLLGNAQQGSQLGSVGSQPNSSAVSNATTNLLNNLTGVMFGNHAAVQPSPVAPVQKPSLGNNTGSGGLNLNNLNPSILAAVVGNLGNQGGNLSNPLLSSSLSNLGLNLGNSGNDDNLPPSNVGLSNNYSSGGTGGGNSYSSGNNYSGGGGSSNLGYNAYSSSGGMGGGNGGVGVDGNDYNTGNPLDVYGGGSNVGNSNVGSANAVGASRKSDTIIIKNVPITCTWQTLRDKFREIGDVKFAEIRGNDVGVVRFFKERDAELAIALMDGSRLDGRNIKV

SEQ ID NO: 55 - protein, 541 aa, Homo sapiens
MENDESAKEEKSDLKEKSTGSKKANRFHPYSKDKNSGTGEKKGPNRNRVFISNIPYDMKWQAIKDLMREKVGEVTYVELFKDAEGKSRGCGVVEFKDEEFVKKALETMNKYDLSGRRVNIKEDPDGENARRALQRTGTSFQGSHASDVGSGLVNLPPSILNNPNIPPEVISNLQAGRLGSTIFVANLDFKVGWKKLKEVFSIAGTVKAGSYKEDKDGKSRGMGTVTFEQAIEAVQAISMFNGQFLFDRPMHVKMDDKSVPHEEYRSPDGKTPQLPRGLGGIGMGLGPGGQPISASQLNIGGVMGNLGPGGMGMDGPGFGGMNRIGGGIGFGGLEAMNSMGGFGGVGRMGELYRGAMTSSMERDFGHRDIGLSRGFGDSFGRLGSAMIGGITGRIGSSNMGPVGSGISGGMGSMNSVTGGMGMGLDRMSSSFDRMGPGIGAILERSIDMDRGFLSGPMGSGMRERIGSKGNQIFVRNLPFDLTWQKLKEKFSQCGHVMFAEIKMENGKSKGCGTVRFDSPESAEKACRIMNGIKISGREIDV

SEQ ID NO: 56 - protein, 108 aa, Homo sapiens; identical to SEQ ID NO: 47

SEQ ID NO: 57 - protein, 157 aa, Drosophila melanogaster
GGGGGGVQGGNGGNNGGGGGGGRDHMDDRDRGFSRRDDDRLSGRNNFNMMSNDYNNSSNYNLYGLSASFLESLGISGPLHNKVFVANLDYKVDNKKLKQVFKLAGKVQSVDLSLDKEGNSRGFAVIEYDHPVEAVQAISMLDRQMLFDRRMTVRLDR

SEQ ID NO: 58 - protein, 146 aa, Homo sapiens
GPVGSGISGGMGSMNSVTGGMGMGLDRMSSSFDRMGPGIGAILERSIDMDRGFLSGPMGSGMRERIGSKGNQIFVRNLPFDLTWQKLKEKFSQCGHVMFAEIKMENGKSKGCGTVRFDSPESAEKACRIMNGIKISGREIDVRLDR

SEQ ID NO: 59 - protein, 632 aa, Drosophila melanogaster
MSMDASNSVESREKERDRRGRGARGSRFTDADGNGNGAGSQGGGVAARDRSRERRNCRVYISNIPYDYRWQDLKDLFRRIVGSIEYVQLFFDESGKARGCGIVEFKDPENVQKALEKMNRYEVNGRELVVKEDHGEQRDQYGRIVRDGGGGGGGGGGVQGGNGGNNGGGGGGGRDHMDDRDRGFSRRDDDRLSGRNNFNMMSNDYNNSSNYNLYGLSASFLESLGISGPLHNKVFVANLDYKVDNKKLKQVFKLAGKVQSVDLSLDKEGNSRGFAVIEYDHPVEAVQAISMLDRQMLFDRRMTVRLDRIPDKNEGIKLPEGLGGVGIGLGPNGEPLRDVAHNLPNGGQSQGQLLGNAQQGSQLGSVGSQPNSSAVSNATTNLLNNLTGVMFGNHAAVQPSPVAPVQKPSLGNNTGSGGLNLNNLNPSILAAVVGNLGNQGGNLSNPLLSSSLSNLGLNLGNSGNDDNLPPSNVGLSNNYSSGGTGGGNSYSSGNNYSGGGGSSNLGYNAYSSSGGMGGGNGGVGVDGNDYNTGNPLDVYGGGSNVGNSNVGSANAVGASRKSDTIIIKNVPITCTWQTLRDKFREIGDVKFAEIRGNDVGVVRFFKERDAELAIALMDGSRLDGRNIKVTYF

SEQ ID NO: 60 - protein, 620 aa, Homo sapiens
PLSRSEPLSSGGRGGGSGGGMADANKAEVPGATGGDSPHLQPAEPPGEPRREPHPAEAEKQQPQHSSSSNGVKMENDESAKEEKSDLKEKSTGSKKANRFHPYSKDKNSGTGEKKGPNRNRVFISNIPYDMKWQAIKDLMREKVGEVTYVELFKDAEGKSRGCGVVEFKDEEFVKKALETMNKYDLSGRPLNIKEDPDGENARRALQRTGGSFPGGHVPDMGSGLMNLPPSILNNPNIPPEVISNLQAGRLGSTIFVANLDFKVGWKKLKEVFSIAGTVKRADIKEDKDGKSRGMGTVTFEQAIEAVQAISMFNGQFLFDRPMHVKMDDKSVPHEEYRSHDGKTPQLPRGLGGIGMGLGPGGQPISASQLNIGGVMGNLGPGGMGMDGPGFGGMNRIGGGIGFGGLEAMNSMGGFGGVGRMGELYRGAMTSSMERDFGRGDIGINRGFGDSFGRLGSAMIGGFAGRIGSSNMGPVGSGISGGMGSMNSVTGGMGMGLDRMSSSFDRMGPGIGAILERSIDMDRGFLSGPMGSGMRERIGSKGNQIFVRNLPFDLTWQKLKEKFSQCGHVMFAEIKMENGKSKGCGTVRFDSPESAEKACRIMNGIKISGREIDVRLDRNA

SEQ ID NO: 61 - protein, 547 aa, Homo sapiens
MENDESAKEEKSDLKEKSTGSKKANRFHPYSKDKNSGTGEKKGPNRNRVFISNIPYDMKWQAIKDLMREKVGEVTYVELFKDAEGKSRGCGVVEFKDEEFVKKALETMNKYDLSGRRVNIKEDPDGENARRALQRTGTSFQGSHASDVGSGLVNLPPSILNNPNIPPEVISNLQAGRLGSTIFVANLDFKVGWKKLKEVFSIAGTVKAGSYKEDKDGKSRGMGTVTFEQAIEAVQAISMFNGQFLFDRPMHVKMDDKSVPHEEYRSPDGKTPQLPRGLGGIGMGLGPGGQPISASQLNIGGVMGNLGPGGMGMDGPGFGGMNRIGGGIGFGGLEAMNSMGGFGGVGRMGELYRGAMTSSMERDFGHRDIGLSRGFGDSFGRLGSAMIGGITGRIGSSNMGPVGSGISGGMGSMNSVTGGMGMGLDRMSSSFDRMGPGIGAILERSIDMDRGFLSGPMGSGMRERIGSKGNQIFVRNLPFDLTWQKLKEKFSQCGHVMFAEIKMENGKSKGCGTVRFDSPESAEKACRIMNGIKISGREIDVRLDRNA

SEQ ID NO: 62 - protein, 576 aa, Homo sapiens [sequence omitted]

SEQ ID NO: 63 - protein, 196 aa, Homo sapiens
MNNGGKAEKENTPSEANLQEEEVRTLFVSGLPLDIKPRELYLLFRPFKGYEGSLIKLTSKQPVGFVSFDSRSEAEAAKNALNGIRFDPEIPQTLRLEFAKANTKMAKNKLVGTPNPSTPLPNTVPQFIAREPYELTVPALYPSSPEVWAPYPLYPAELAPALPPPAFTYPASLHAQMRWLPPSEATSQGWKSRQFC

SEQ ID NO: 64 - protein, 168 aa, Homo sapiens
QVRTLFVSGLPVDIKPRELYLLFRPFKPVGFVIFDSRAGAEAAKNALNGIRFDPENPQTLRLEFAKANTKMAKSKLMATPNPSNVHPALGAHFIARDPYDLMGAALIPASPEAWAPYPLYTTELTPAISHAAFTYPTATAAAAALHAQVRWYPSSDTTQQGWKYRQFC

SEQ ID NO: 65 - protein, 188 aa, Drosophila melanogaster
QLQKPAPAFAGTAVVNGVFKDIKLSDYKGKYLVLFFYPLDFTFVCPTEIIAFSESAAEFRKINCEVIGCSTDSQFTHLAWINTPRKQGGLGSMDIPLLADKSMKVARDYGVLDEETGIPFRGLFIIDDKQNLRQITVNDLPVGRSVEETLRLVQAFQYTDKYGEVCPANWKPGQKTMVADPTKSKEYF

SEQ ID NO: 66 - protein, 188 aa, Homo sapiens
RIGKPAPDFKATAVVDGAFKEVKLSDYKGKYVVLFFYPLDFTFVCPTEIIAFSNRAEDFRKLGCEVLGVSVDSQFTHLAWINTPRKEGGLGPLNIPLLADVTRRLSEDYGVLKTDEGIAYRGLFIIDGKGVLRQITVNDLPVGRSVDEALRLVQAFQYTDEHGEVCPAGWKPGSDTIKPNVDDSKEYF

SEQ ID NO: 67 - protein, 184 aa, Drosophila melanogaster
PAPAFAGTAVVNGVFKDIKLSDYKGKYLVLFFYPLDFTFVCPTEIIAFSESAAEFRKINCEVIGCSTDSQFTHLAWINTPRKQGGLGSMDIPLLADKSMKVARDYGVLDEETGIPFRGLFIIDDKQNLRQITVNDLPVGRSVEETLRLVQAFQYTDKYGEVCPANWKPGQKTMVADPTKSKEYF

SEQ ID NO: 68 - protein, 185 aa, Homo sapiens
PAPNFKATAVMPDGQFKDISLSDYKGKYVVFFFYPLDFTFVCPTEIIAFSDRAEEFKKLNCQVIGASVDSHFCHLAWVNTPKKQGGLGPMNIPLVSDPKRTIAQDYGVLKADEGISFRGLFIIDDKGILRQITVNDLPVGRSVDETLRLVQAFQFTDKHGEVCPAGWKPGSDTIKPDVQKSKEYF

SEQ ID NO: 69 - protein, 194 aa, Drosophila melanogaster
MPQLQKPAPAFAGTAVVNGVFKDIKLSDYKGKYLVLFFYPLDFTFVCPTEIIAFSESAAEFRKINCEVIGCSTDSQFTHLAWINTPRKQGGLGSMDIPLLADKSMKVARDYGVLDEETGIPFRGLFIIDDKQNLRQITVNDLPVGRSVEETLRLVQAFQYTDKYGEVCPANWKPGQKTMVADPTKSKEYFETTS

SEQ ID NO: 70 - protein, 199 aa, Homo sapiens
MSSGNAKIGHPAPNFKATAVMPDGQFKDISLSDYKGKYVVFFFYPLDFTFVCPTEIIAFSDRAEEFKKLNCQVIGASVDSHFCHLAWVNTPKKQGGLGPMNIPLVSDPKRTIAQDYGVLKADEGISFRGLFIIDDKGILRQITVNDLPVGRSVDETLRLVQAFQFTDKHGEVCPAGWKPGSDTIKPDVQKSKEYFSKQK

SEQ ID NO: 71 - protein, 198 aa, Homo sapiens
MASGNARIGKPAPDFKATAVVDGAFKEVKLSDYKGKYVVLFFYPLDFTFVCPTEIIAFSNRAEDFRKLGCEVLGVSVDSQFTHLAWINTPRKEGGLGPLNIPLLADVTRRLSEDYGVLKTDEGIAYRGLFIIDGKGVLRQITVNDLPVGRSVDEALRLVQAFQYTDEHGEVCPAGWKPGSDTIKPNVDDSKEYFSKHN

SEQ ID NO: 72 - protein, 106 aa, Drosophila melanogaster
QGQSSRAQKAARRRSNESIEARERRLERNAARMRDKRAKESEAEYRVRLAKNAEANRVRRQNETEVQRTLRLMKNAARQRLRRASETVEERKKRLAKAAERMRIAR

SEQ ID NO: 73 - protein, 106 aa, Homo sapiens
EAQTPSVRKWALRRQNEPLEVRLQRLERERTAKKSRRDNETPEEREVRRMRDREAKRLQRMQETDEQRARRLQRDREAMRLKRANETPEKRQARLIREREAKRLKR